SEQUENCE LISTING (33 sequences)

SEQ ID NO: 1 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 2 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 3 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 4 - protein, 157 aa, Homo sapiens
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 5 - protein, 193 aa, Homo sapiens
MAAEPVEDNCINFVAMKFIDNTLYFIAEDDENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 6 - protein, 486 aa, synthetic construct
  REGION 1..486: note = Description of Artificial Sequence: CD 19 binding peptide
  VARIANT 486: note = X is any amino acid
MALPVTALLLPLALLLHAEVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRX

SEQ ID NO: 7 - DNA, 1458 bp, synthetic construct
  misc_feature 1..1458: note = Description of Artificial Sequence: Synthetic polynucleotide
  source 1..1458: mol_type = other DNA
ccatggctctcccagtgactgccctactgcttcccctagcgcttctcctgcatgcagaggtgaagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggagatggtgatactaactacaatggaaagttcaagggtcaagccacactgactgcagacaaatcctccagcacagcctacatgcagctcagcggcctaacatctgaggactctgcggtctatttctgtgcaagaaagaccattagttcggtagtagatttctactttgactactggggccaagggaccacggtcaccgtctcctcaggtggaggtggatcaggtggaggtggatctggtggaggtggatctgacattgagctcacccagtctccaaaattcatgtccacatcagtaggagacagggtcagcgtcacctgcaaggccagtcagaatgtgggtactaatgtagcctggtatcaacagaaaccaggacaatctcctaaaccactgatttactcggcaacctaccggaacagtggagtccctgatcgcttcacaggcagtggatctgggacagatttcactctcaccatcactaacgtgcagtctaaagacttggcagactatttctgtcaacaatataacaggtatccgtacacgtccggaggggggaccaagctggagatcaaacgggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagagccccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcg

SEQ ID NO: 8 - protein, 20 aa, Homo sapiens
MYRMQLLSCIALSLALVTNS

SEQ ID NO: 9 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 10 - protein, 247 aa, Mus musculus
MASPLTRFLSLNLLLMGESIILGSGEAKPQAPELRIFPKKMDAELGQKVDLVCEVLGSVSQGCSWLFQNSSSKLPQPTFVVYMASSHNKITWDEKLNSSKLFSAVRDTNNKYVLTLNKFSKENEGYYFCSVISNSVMYFSSVVPVLQKVNSTTTKPVLRTPSPVHPTGTSQPQRPEDCRPRGSVKGTGLDFACDIYIWAPLAGICVAPLLSLIITLICYHRSRKRVCKCPRPLVRQEGKPRPSEKIV

SEQ ID NO: 11 - protein, 69 aa, Mus musculus
STTTKPVLRTPSPVHPTGTSQPQRPEDCRPRGSVKGTGLDFACDIYIWAPLAGICVALLLSLIITLICY

SEQ ID NO: 12 - DNA, 207 bp, Mus musculus
  source 1..207: mol_type = genomic DNA
tctactactaccaagccagtgctgcgaactccctcacctgtgcaccctaccgggacatctcagccccagagaccagaagattgtcggccccgtggctcagtgaaggggaccggattggacttcgcctgtgatatttacatctgggcacccttggccggaatctgcgtggcccttctgctgtccttgatcatcactctcatctgctac

SEQ ID NO: 13 - protein, 188 aa, Mus musculus
MKWKVSVLACILHVRFPGAEAQSFGLLDPKLCYLLDGILFIYGVIITALYLRAKFSRSAETAANLQDPNQLYNELNLGRREEYDVLEKKRARDPEMGGKQRRRNPQEGVYNALQKDKMAEAYSEIGTKGERRRGKGHDGLYQDSHFQAVQFGNRREREGSELTRTLGLRARPKACRHKKPLSLPAAVS

SEQ ID NO: 14 - protein, 113 aa, Mus musculus
RAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 15 - DNA, 342 bp, Mus musculus
  source 1..342: mol_type = genomic DNA
agagcaaaattcagcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcccctcgctaa

SEQ ID NO: 16 - protein, 218 aa, Mus musculus
MTLRLLFLALNFFSVQVTENKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRP

SEQ ID NO: 17 - DNA, 123 bp, Mus musculus
  source 1..123: mol_type = genomic DNA
aatagtagaaggaacagactccttcaaagtgactacatgaacatgactccccggaggcctgggctcactcgaaagccttaccagccctacgcccctgccagagactttgcagcgtaccgcccc

SEQ ID NO: 18 - protein, 277 aa, Homo sapiens
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 19 - protein, 199 aa, synthetic construct
  REGION 1..199: note = Description of Artificial Sequence: Synthetic polypeptide
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 20 - protein, 491 aa, synthetic construct
  REGION 1..491: note = Description of Artificial Sequence: Synthetic polypeptide
MALPVTALLLPLALLLHAEVKLQESGGGFVKPGGSLKVSCAASGFTFSSYAMSWVRLSPEMRLEWVATISSAGGYIFYSDSVQGRFTISRDNAKNTLHLQMGSLRSGDTAMYYCARQGFGNYGDYYAMDYWGQGTTVTVSSGGGGSGGGGSGGGGSDIELTQSPSSLAVSAGEKVTMSCKSSQSLLNSRTRKNQLAWYQQKPGQSPELLIYWASTRQSGVPDRFTGSGSGTDFTLTISSVQAEDLAVYYCQQSYNLLTFGPGTKLEIKRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - DNA, 1475 bp, synthetic construct
  misc_feature 1..1475: note = Description of Artificial Sequence: Synthetic polynucleotide
  source 1..1475: mol_type = other DNA
ccatggctctcccagtgactgccctactgcttcccctagcgcttctcctgcatgcagaggtgaagctgcaggagtcagggggaggcttcgtgaagcctggagggtccctcaaagtctcctgtgcagcctctggattcactttcagtagctatgccatgtcctgggttcgcctgagtccggagatgaggctggagtgggtcgcaaccattagcagtgctggtggttacatcttctattctgacagtgtgcagggacgattcaccatttccagagacaatgccaagaacaccctgcacctgcaaatgggcagtctgaggtctggggacacggccatgtattactgtgcaaggcagggatttggtaactacggtgattactatgctatggactactggggccaagggaccacggtcaccgtctcctcaggtggaggtggatcaggtggaggtggatctggtggaggtggatctgacattgagctcacccagtctccatcctccctggctgtgtcagcaggagagaaggtcactatgagctgcaaatccagtcagagtctgctcaacagtagaacccgaaagaaccagttggcttggtaccagcaaaaaccaggacagtctcctgaactgctgatctactgggcatccactaggcaatctggagtccctgatcgcttcacaggcagtggatctgggacagatttcactctcaccatcagcagtgtgcaggctgaagacctggcagtttattactgccagcaatcttataatctactcacgttcggtcctgggaccaagctggagatcaaacgggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagagccccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 22 - DNA, 81 bp, Homo sapiens
  source 1..81: mol_type = genomic DNA
ttttgggtgctggtggtggttggtggagtcctggcttgctatagcttgctagtaacagtggcctttattattttctgggtg

SEQ ID NO: 23 - protein, 15 aa, synthetic construct
  REGION 1..15: note = Description of Artificial Sequence: Synthetic peptide
GGGGSGGGGSGGGGS

SEQ ID NO: 24 - protein, 20 aa, Mus musculus
MYSMQLASCVTLTLVLLVNS

SEQ ID NO: 25 - protein, 20 aa, Homo sapiens
METPAQLLFLLLLWLPDTTG

SEQ ID NO: 26 - protein, 20 aa, Mus musculus
METDTLLLWVLLLWVPGSTG

SEQ ID NO: 27 - protein, 21 aa, Homo sapiens
MALPVTALLLPLALLLHAARP

SEQ ID NO: 28 - protein, 16 aa, Homo sapiens
MKWVTFISLLFSSAYS

SEQ ID NO: 29 - protein, 30 aa, Homo sapiens
MDSKGSSQKGSRLLLLLVVSNLLLCQGVVS

SEQ ID NO: 30 - DNA, 1476 bp, synthetic construct
  misc_feature 1..1476: note = Description of Artificial Sequence: Synthetic polynucleotide
  source 1..1476: mol_type = other DNA
atggctctcccagtgactgccctactgcttcccctagcgcttctcctgcatgcagaggtgaagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggagatggtgatactaactacaatggaaagttcaagggtcaagccacactgactgcagacaaatcctccagcacagcctacatgcagctcagcggcctaacatctgaggactctgcggtctatttctgtgcaagaaagaccattagttcggtagtagatttctactttgactactggggccaagggaccacggtcaccgtctcctcaggtggaggtggatcaggtggaggtggatctggtggaggtggatctgacattgagctcacccagtctccaaaattcatgtccacatcagtaggagacagggtcagcgtcacctgcaaggccagtcagaatgtgggtactaatgtagcctggtatcaacagaaaccaggacaatctcctaaaccactgatttactcggcaacctaccggaacagtggagtccctgatcgcttcacaggcagtggatctgggacagatttcactctcaccatcactaacgtgcagtctaaagacttggcagactatttctgtcaacaatataacaggtatccgtacacgtccggaggggggaccaagctggagatcaaacgggcggccgcacccaccacgacgccagcgccgcgaccaccaaccccggcgcccacgatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccctttactgcaacaaacggggcagaaagaagctcctgtatatattcaaacaaccatttatgagaccagtacaaactactcaagaggaagatggctgtagctgccgatttccagaagaagaagaaggaggatgtgaactgagagtgaagttcagcaggagcgcagagccccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 31 - DNA, 2198 bp, synthetic construct
  misc_feature 1..2198: note = Description of Artificial Sequence: Synthetic polynucleotide
  source 1..2198: mol_type = other DNA
atggctctcccagtgactgccctactgcttcccctagcgcttctcctgcatgcagaggtgaagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggagatggtgatactaactacaatggaaagttcaagggtcaagccacactgactgcagacaaatcctccagcacagcctacatgcagctcagcggcctaacatctgaggactctgcggtctatttctgtgcaagaaagaccattagttcggtagtagatttctactttgactactggggccaagggaccacggtcaccgtctcctcaggtggaggtggatcaggtggaggtggatctggtggaggtggatctgacattgagctcacccagtctccaaaattcatgtccacatcagtaggagacagggtcagcgtcacctgcaaggccagtcagaatgtgggtactaatgtagcctggtatcaacagaaaccaggacaatctcctaaaccactgatttactcggcaacctaccggaacagtggagtccctgatcgcttcacaggcagtggatctgggacagatttcactctcaccatcactaacgtgcagtctaaagacttggcagactatttctgtcaacaatataacaggtatccgtacacgtccggaggggggaccaagctggagatcaaacgggcggccgcacccaccacgacgccagcgccgcgaccaccaaccccggcgcccacgatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccctttactgcaacaaacggggcagaaagaagctcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggatctggagcaacaaacttctcactactcaaacaagcaggtgacgtggaggagaatcccggacccatgggttacaggatgcaactcctgtcttgcattgcactaagtcttgcacttgtcacaaacagtggctactttggcaagcttgaatctaaattatcagtcataagaaatttgaatgaccaagttctcttcattgaccaaggaaatcggcctctatttgaagatatgactgattctgactgtagagataatgcaccccggaccatatttattataagtatgtataaagatagccagcctagaggtatggctgtaactatctctgtgaagtgtgagaaaatttcaactctctcctgtgagaacaaaattatttcctttaaggaaatgaatcctcctgataacatcaaggatacaaaaagtgacatcatattctttcagagaagtgtcccaggacatgataataagatgcaatttgaatcttcatcatacgaaggatactttctagcttgtgaaaaagagagagacctttttaaactcattttgaaaaaagaggatgaattgggggatagatctataatgttcactgttcaaaacgaagactaggtcgaggatccggattagtccaatttgttaaagacaggatatcagtggtccaggctctagttttgactcaacaatatcaccagctgaagcctatagagtacgagccatagataaaataa

SEQ ID NO: 32 - DNA, 548 bp, Homo sapiens
  source 1..548: mol_type = genomic DNA
ccatgggttacaggatgcaactcctgtcttgcattgcactaagtcttgcacttgtcacaaacagtggctactttggcaagcttgaatctaaattatcagtcataagaaatttgaatgaccaagttctcttcattgaccaaggaaatcggcctctatttgaagatatgactgattctgactgtagagataatgcaccccggaccatatttattataagtatgtataaagatagccagcctagaggtatggctgtaactatctctgtgaagtgtgagaaaatttcaactctctcctgtgagaacaaaattatttcctttaaggaaatgaatcctcctgataacatcaaggatacaaaaagtgacatcatattctttcagagaagtgtcccaggacatgataataagatgcaatttgaatcttcatcatacgaaggatactttctagcttgtgaaaaagagagagacctttttaaactcattttgaaaaaagaggatgaattgggggatagatctataatgttcactgttcaaaacgaagactagggatcc

SEQ ID NO: 33 - DNA, 728 bp, Mus musculus
  source 1..728: mol_type = genomic DNA
ctcgagggtagcggtgccactaacttcagtctccttaagcaggctggcgatgtggaagaaaatcctggaccatccatgggttacaggatgcaactcctgtcttgcattgcactaagtcttgcacttgtcacaaacagtggcgctgccatgtcagaagactcttgcgtcaacttcaaggaaatgatgtttattgacaacacgctttactttatacctgaagaaaatggagacctggaatcagacaactttggccgacttcactgtacaaccgcagtaatacggaatataaatgaccaagttctcttcgttgacaaaagacagcctgtgttcgaggatatgactgatattgatcaaagtgccagtgaaccccagaccagactgataatatacatgtacaaagacagtgaagtaagaggactggctgtgaccctctctgtgaaggatagtaaaatgtctaccctctcctgtaagaacaagatcatttcctttgaggaaatggacccacctgaaaatattgatgatatacaaagtgatctcatattctttcagaaacgtgttccaggacacaacaagatggagtttgaatcttcactgtatgaaggacactttcttgcttgccaaaaggaagatgatgctttcaaactcattctgaaaaaaaaggatgaaaatggggataaatctgtaatgttcactctcactaacttacatcaaagttagctcgaggatcc

(19) United States
(12) Patent Application Publication
(10) Pub. No.: US 2024/0307441 A1
(43) Pub. Date: Sep. 19, 2024

(54) COMPOSITIONS AND METHODS FOR IMMUNOTHERAPY

(71) Applicant: Memorial Sloan-Kettering CAncer Center, New York, NY (US)

(72) Inventors: Renier J. Brentjens, Short Hills, NJ (US); Mauro P. Avanzi, New York, NY (US); Sarah Yoo, Great Neck, NY (US)

(73) Assignee: Memorial Sloan-Kettering Cancer Center, New York, NY (US)

(21) Appl. No.: 18/538,640

(22) Filed: Dec. 13, 2023

Related U.S. Application Data
(62) Division of application No. 16/265,168, filed on Feb. 1, 2019, now abandoned.
Application No. 16/265,168 is a continuation of application No. PCT/US17/45550, filed on Aug. 4, 2017.
(60) Provisional application No. 62/370,969, filed on Aug. 4, 2016.

(51) Int. Cl. A61K 35/17; A61K 38/20; A61P 35/00; C07K 14/54; C07K 14/705; C07K 14/725; C07K 16/28; C12N 5/0783

(52) U.S. Cl. CPC A61K 35/17; A61K 38/20; A61P 35/00; C07K 14/54; C07K 14/7051; C07K 14/70521; C07K 16/2803; C12N 5/0636; C07K 2319/02; C07K 2319/03; C12N 2501/2318; C12N 2510/02

(57) ABSTRACT

The present disclosure provides methods and compositions for enhancing the immune response toward cancers and pathogens. It relates to immunoresponsive cells comprising antigen recognizing receptors (e.g., chimeric antigen receptors (CARs) or T cell receptors (TCRs)), and expressing increased level of IL-18. In certain embodiments, the engineered immunoresponsive cells are antigen-directed and resistant to immunosuppression and/or have enhanced immune-activating properties.

### CROSS-REFERENCE TO RELATED APPLICATION

This application is a Divisional of U.S. patent application Ser. No. 16/265,168, filed Feb. 1, 2019, which is a Continuation of International Patent Application No. PCT/US2017/045550, filed Aug. 4, 2017, which claims priority to U.S. Provisional Application No. 62/370,969 filed on Aug. 4, 2016, the contents of each of which are hereby incorporated by reference in their entireties, and to each of which priority is claimed.

### SEQUENCE LISTING

The specification further incorporates by reference the Sequence Listing submitted herewith via Patent Center on Dec. 13, 2023. The Sequence Listing XML file, identified as 072734.1534.xml, is 45,243 bytes and was created on Dec. 13, 2023. The Sequence Listing, electronically filed herewith, does not extend beyond the scope of the specification and thus does not contain new matter.

### INTRODUCTION

The presently disclosed subject matter provides methods and compositions for enhancing the immune response toward cancers and pathogens. It relates to immunoresponsive cells comprising antigen recognizing receptors (e.g., chimeric antigen receptors (CARs) or T cell receptors (TCRs) that are engineered to express IL-18. These engineered immunoresponsive cells are antigen-directed, promote recruitment of other cytokines and exhibit enhanced anti-target efficacy.

### BACKGROUND OF THE INVENTION

The majority of adult B-cell malignancies, including acute lymphoblastic leukemia (ALL), chronic lymphocytic leukemia, and non-Hodgkin's lymphoma, are incurable despite currently available therapies. Adoptive therapy with genetically engineered autologous T cells has shown evidence of therapeutic efficacy in melanoma and indolent B cell malignancies. T cells may be modified to target tumor-associated antigens through the introduction of genes encoding artificial T-cell receptors, termed chimeric antigen receptors (CAR), specific to such antigens. Immunotherapy is a targeted therapy that has the potential to provide for the treatment of cancer.

However, malignant cells adapt to generate an immunosuppressive microenvironment to protect themselves from immune recognition and elimination. This “hostile” tumor microenvironment poses a challenge to methods of treatment involving stimulation of an immune response, such as targeted T cell therapies. Various modifications have been made toward improving the antitumor effect of CAR- or TCR-engineered T cells. For example, Pegram et al. describes a murine model of CAR-engineered T cells that constitutively secrete interleukin 12 (IL-12) and showed increased cytotoxicity towards CD19+ tumor cells (Pegram et al., BLOOD, Vol. 119, No. 18, 2012). However, the secretion of IL-12 led to suppression of interleukin 2 (IL-2), an important cytokine that promotes the proliferation and anti-tumor effect of T and B lymphocytes. Dotti et al. discloses CAR-engineered T cells that constitutively secrete interleukin 15 (IL-15) and an inducible caspase-9 based suicide gene (iC9), which showed increase cytotoxicity towards CD19⁺ tumor cells (US 20130071414 A1). This modified CAR-T cell demonstrated unchanged levels of IL-2 expression both in vivo and in vitro. Accordingly, novel therapeutic strategies for treating neoplasia are urgently required.

### SUMMARY OF THE INVENTION

The presently disclosed subject matter provides immunoresponsive cells (e.g., T cells, Tumor Infiltrating Lymphocytes, Natural Killer (NK) cells, cytotoxic T lymphocytes (CTLs), Natural Killer T (NK-T) cells or regulatory T cells) which (a) express an antigen recognizing receptor (e.g., CAR or TCR) directed toward a target antigen of interest, and (b) express and secrete interleukin 18 (“IL-18”) (generally, immunoresponsive/IL-18 expressing cell, or “IR/IL-18 cell”). In certain non-limiting embodiments, the cell comprises an exogenous IL-18 polypeptide-encoding nucleic acid, in expressible form. In certain non-limiting embodiments, the cell expresses (a) an antigen recognizing receptor (e.g., a CAR or TCR) directed toward a target antigen of interest and (b) IL-18 polypeptide, for example via an exogenous IL-18 polypeptide encoding nucleic acid in expressible form. In certain embodiments, the cell constitutively expresses the IL-18 polypeptide (mature or non-mature form of IL-18). In certain embodiments, the IL-18 polypeptide is secreted. In certain embodiments, the antigen recognizing receptor is a T cell receptor (TCR) or chimeric antigen receptor (CAR). In certain embodiments, the antigen recognizing receptor is a CAR, and the cell is a CAR/IL-18 T cell (“T-CAR/IL-18”). In certain embodiments, the cell is selected from the group consisting of a T cell, a Natural Killer (NK) cell, a cytotoxic T lymphocyte (CTL), a regulatory T cell, a Natural Killer T (NK-T) cell, a human embryonic stem cell, and a pluripotent stem cell from which lymphoid cells may be differentiated. In certain embodiments, the cell is autologous.

Furthermore, the presently disclosed subject matter provides methods of using such immunoresponsive cells for treating and/or preventing a neoplasia (e.g., cancer), infectious disease, and other pathologies that would benefit from an augmented immune response.

In certain non-limiting embodiments, the presently disclosed subject matter provides an isolated immunoresponsive cell comprising an antigen recognizing receptor that binds to an antigen (CAR or TCR), and constitutively expressing IL-18 polypeptide. In certain embodiments, the immunoresponsive cell comprises an exogenous IL-18 polypeptide. In certain embodiments, binding of the antigen recognizing receptor to the antigen is capable of activating the immunoresponsive cell.

The presently disclosed subject matter also provides a method of treating and/or preventing a neoplasia in a subject, the method comprising administering, to the subject, an effective amount of immunoresponsive cells disclosed herein. The presently disclosed subject matter also provides a method of reducing tumor burden in a subject, the method comprising administering, to the subject, an effective amount of immunoresponsive cells disclosed herein. The presently disclosed subject matter further provides a method of lengthening survival of a subject having neoplasia (e.g., cancer), the method comprising administering, to the subject, an effective amount of immunoresponsive cells disclosed herein.

The presently disclosed subject matter also provides a method of increasing an immune response to a target antigen in a subject, comprising administering, to the subject, an effective amount of immunoresponsive cells disclosed herein, wherein said cell produces and secretes IL-18 polypeptide that enhances the subject's immune response toward the target antigen.

The presently disclosed subject matter further provides a method of increasing immune-activating cytokine production in response to a cancer or pathogen in a subject, comprising administering, to the subject, an effective amount of immunoresponsive cells disclosed herein. In certain non-limiting embodiments, the immune-activating cytokine is selected from the group consisting of IL-2, TNF-α and IFN-γ. In certain non-limiting embodiments, the immune-activating cytokine is IL-2.

The presently disclosed subject matter further provides a method of increasing a CD8⁺ cytotoxic T cell response to a cancer cell or a pathogen in a subject, comprising administering, to the subject, an effective amount of immunoresponsive cells disclosed herein.

The presently disclosed subject matter further provides a method of promoting dendritic cell maturation in a subject having a cancer or a disease caused by a pathogen, comprising administering, to the subject, an effective amount of immunoresponsive cells disclosed herein.

The presently disclosed subject matter further provides a method of treating blood cancer in a subject in need thereof, the method involving administering to the subject a therapeutically effective amount of the immunoresponsive cells disclosed herein, wherein the cells are T cells comprising an antigen recognizing receptor that binds CD19.

The presently disclosed subject matter further provides a method for producing an immunoresponsive cell disclosed herein, the method involving introducing into the immunoresponsive cell a nucleic acid sequence that encodes an IL-18 polypeptide that enhances an immune response of the immunoresponsive cell, where the immunoresponsive cell has an antigen recognizing receptor that binds an antigen.

In certain non-limiting embodiments, the nucleic acid sequence that encodes the IL-18 polypeptide is operably linked to a promoter element constitutively or inducibly expressed in the immunoresponsive cell, optionally comprised in a vector. In certain non-limiting embodiments, the antigen recognizing receptor is a CAR.

The presently disclosed subject matter further provides a nucleic acid comprising a first nucleic acid sequence encoding an antigen recognizing receptor (e.g., a CAR or TCR) and a second nucleic acid sequence encoding an IL-18 polypeptide (mature or non-mature form of IL-18), each optionally operably linked to a promoter element constitutively or inducibly expressed in the immunoresponsive cell. In certain embodiments, said nucleic acid may optionally be comprised in a vector. The presently disclosed subject matter also provides a vector comprising such nucleic acid. Optionally one or both nucleic acids may be comprised in a vector, which may be the same vector (bicistronic) or separate vectors. The nucleic acid encoding the antigen recognizing receptor (e.g., a CAR or a TCR) and/or the nucleic acid encoding the IL-18 polypeptide may each be operably linked to a promoter which may be the same or different promoters. The antigen receptor (e.g. CAR)-encoding nucleic acid and the IL-18 polypeptide-encoding nucleic acid may be separate molecules each optionally comprised in a separate vector. In certain non-limiting embodiments, the vector is a virus vector, e.g., a retroviral vector.

The presently disclosed subject matter further provides a pharmaceutical composition comprising an effective amount of immunoresponsive cells disclosed herein and a pharmaceutically acceptable excipient. In certain embodiments, the pharmaceutical composition is a pharmaceutical composition for treating and/or a neoplasia (e.g., cancer), wherein the antigen to which the antigen recognizing receptor binds is a tumor antigen.

The presently disclosed subject matter provides a kit for treating and/or preventing a neoplasia (e.g., cancer) or, pathogen infection, the kit comprising immunoresponsive cells disclosed herein, a nucleic acid disclosed herein, or a vector disclosed herein. In certain embodiments, the kit further comprises written instructions for treating and/or preventing a neoplasia or a pathogen infection.

In various non-limiting embodiments, the immunoresponsive cell is autologous to its intended recipient subject.

In various embodiments of any of the aspects delineated herein, the antigen recognizing receptor is a T cell receptor (TCR) or a chimeric antigen receptor (CAR). In various embodiments of any of the aspects delineated herein, the antigen recognizing receptor is exogenous or endogenous. In various embodiments of any of the aspects delineated herein, the antigen recognizing receptor is recombinantly expressed. In various embodiments of any of the aspects delineated herein, the antigen recognizing receptor is expressed from a vector. In various embodiments of any of the aspects delineated herein, the antigen recognizing receptor is a CAR comprising an intracellular signaling domain that is the CD3ζ-chain, CD97, CD11a-CD18, CD2, ICOS, CD27, CD154, CD8, OX40, -IBB, CD28, SLAM, ITAM signaling domain, a portion thereof, or combinations thereof. In certain non-limiting embodiments, the antigen recognizing receptor is a CAR comprising at least a portion of CD28, 4-1BB, ICOS and/or CD3ζ-chain (see, e.g., Zhong et al., 2010, Molecular Ther. 18(2):413-420), together with an antigen binding portion. In certain non-limiting embodiments, the antigen recognizing receptor is a CAR described in Kohn et al., 2011, Molecular Ther. 19(3):432-438), optionally where the antigen binding portion is substituted with amino acid sequence that binds to another tumor or pathogen antigen. In various embodiments, the CAR is 1928z, 19BBz, or 4H1128z.

In various embodiments of any of the aspects delineated herein, the antigen recognizing receptor is a T cell receptor (TCR). In certain embodiments, the TCR is a recombinant TCR. In certain embodiments, the TCR is a non-naturally occurring TCR. In certain embodiments, the TCR differs from any naturally occurring TCR by at least one amino acid residue. In certain embodiments, the TCR is modified from a naturally occurring TCR by at least one amino acid residue.

In various embodiments of any of the aspects delineated herein, the antigen is a tumor or pathogen antigen. In various embodiments of any of the aspects delineated herein, the tumor antigen is selected from the group consisting of CD19, MUC16, MUC1, CA1X, CEA, CD8, CD7, CD10, CD20, CD22, CD30, CD33, CLL1 CD34, CD38, CD41, CD44, CD49f, CD56, CD74, CD133, CD138, a cytomegalovirus (CMV) infected cell antigen, EGP-2, EGP-40, EpCAM, erb-B2,3,4, FBP, Fetal acetylcholine receptor, folate receptor-α, GD2, GD3, HER-2, hTERT, IL-13R-a2, κ-light chain, KDR, LeY, L1 cell adhesion molecule, MAGE-A1, Mesothelin, ERBB2, MAGEA3, p53, MART1,GP100, Proteinase3 (PRI), Tyrosinase, Survivin, hTERT, EphA2, NKG2D ligands, NY-ES0-1, oncofetal antigen (hST4), PSCA, PSMA, ROR1, TAG-72, VEGF-R2, WT-1, BCMA, CD123, CD44V6, NKCS1, EGF1R, EGFR-VIII, or ERBB. In certain embodiments, the antigen is CD19 or MUC16. Amino acid sequences that specifically bind to said antigens are known in the art or may be prepared using methods known in the art; examples include immunoglobulins, variable regions of immunoglobulins (e.g. variable fragment (“Fv”) or bivalent variable fragment (“Fab”)), single chain antibodies, etc.

In various non-limiting embodiments of any of the aspects delineated herein, the exogenous IL-18 polypeptide is secreted. In various non-limiting embodiments of any of the aspects delineated herein, the IL-18 polypeptide is expressed from a vector. In various non-limiting embodiments of any of the aspects delineated herein, the IL-18 polypeptide comprises a heterologous signal sequence at the amino-terminus (that is to say, a signal sequence that is not naturally associated with IL-18). In various embodiments of any of the aspects delineated herein, the heterologous signal sequence is selected from the group consisting of IL-2 signal sequence, the kappa leader sequence, the CD8 leader sequence, and combinations and/or synthetic variations thereof which retain the capacity to promote secretion of IL-18 polypeptide (either mature or non-mature). In various embodiments of any of the aspects delineated herein, the IL-18 polypeptide enhances an immune response of the immunoresponsive cell. In certain embodiments, the exogenous IL-18 polypeptide increases anti-tumor cytokine production. In certain embodiments, the anti-tumor cytokine is selected from the group consisting of IL-2, INF-α and IFN-γ. In certain embodiments, the exogenous IL-18 polypeptide decreases the secretion of cytokines associated with cytokine release syndrome (CRS). In certain embodiments, the cytokines associated with cytokine release syndrome (CRS) is IL-6.

In various non-limiting embodiments of any of the aspects delineated herein, the immunoresponsive cell exhibits enhanced cell expansion compared to an immunoresponsive cell expressing the antigen recognizing receptor alone. In various non-limiting embodiments of any of the aspects delineated herein, the immunoresponsive cell exhibits enhanced cell persistence compared to an immunoresponsive cell expressing the antigen recognizing receptor alone. In various non-limiting embodiments of any of the aspects delineated herein, the immunoresponsive cell induces prolonged B-cell aplasia compared to an immunoresponsive cell expressing the antigen recognizing receptor alone. In various non-limiting embodiments of any of the aspects delineated herein, the immunoresponsive cell activates an endogenous immune cell. In certain embodiments, the endogenous immune cell is selected from the group consisting of a NK cell, a NKT cell, a dendritic cell, a macrophage and an endogenous CD8 T cell. In certain embodiments, the endogenous immune cell is an endogenous CD8 T cells with a central memory phenotype (CD44⁺;Ly6C⁺), a macrophage with an M1 phenotype (MHC-II⁺) or a dendritic cell with a mature and activated phenotype (CD86⁺;MHC-II⁺). In various non-limiting embodiments of any of the aspects delineated herein, the immunoresponsive cell increases the endogenous immune cell population. In various non-limiting embodiments of any of the aspects delineated herein the immunoresponsive cell recruits the endogenous immune cell to a tumor site.

In various embodiments of any of the aspects delineated herein, the immunoresponsive cell secretes a cytokine. In various embodiments of any of the aspects delineated herein, the cytokine is expressed from a vector.

In various non-limiting embodiments of any of the aspects delineated herein, the immunoresponsive cells are administered in a treatment protocol that lacks one or more, or all, of the following: prior conditioning of the host with total body irradiation, high-dose chemotherapy, and/or post-infusion cytokine support. In various subsets of such embodiments, the protocol lacks administration, except as a consequence of treatment with the immunoresponsive cells, of one or more of IL-2, IL-3, IL-6, IL-11, IL-7, IL-12, IL-15, IL-21, granulocyte macrophage colony stimulating factor, alpha, beta or gamma interferon and erythropoietin.

In various non-limiting alternative embodiments of any of the aspects delineated herein, the immunoresponsive cells are administered in a treatment protocol together with one or more, or all, of the following: prior conditioning of the host with total body irradiation, high-dose chemotherapy, and/or post-infusion cytokine support. In various subsets of such alternative embodiments, the protocol comprises administration of one or more of IL-2, IL-3, IL-6, IL-11, IL-7, IL-12, IL-15, IL-21, granulocyte macrophage colony stimulating factor, alpha, beta or gamma interferon and erythropoietin. Accordingly, the presently disclosed subject matter provides a pharmaceutical composition comprising immunoresponsive cells disclosed herein and a cytokine, for example, but not limited to, one or more of IL-2, IL˜3, IL-6, IL-11, IL-7, IL-12, IL-15, IL-21, granulocyte macrophage colony stimulating factor, alpha, beta or gamma interferon and erythropoietin.

In various embodiments of any of the aspects delineated herein, the method reduces the number of tumor cells, reduces tumor size, eradicates the tumor in the subject, reduces the tumor burden in the subject, eradicates the tumor burden in the subject, increases the period of time to relapse/recurrence, and/or increases the period of survival.

Illustrative neoplasms for which the presently disclosed subject matter can be used include, but are not limited to leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma).

In various non-limiting embodiments of any of the aspects delineated herein, the neoplasia is one or more of blood cancer, B cell leukemia, multiple myeloma, lymphoblastic leukemia (ALL), chronic lymphocytic leukemia, non-Hodgkin's lymphoma, and ovarian cancer. In certain embodiments, the blood cancer is one or more of B cell leukemia, multiple myeloma, acute lymphoblastic leukemia (ALL), chronic lymphocytic leukemia, and non-Hodgkin's lymphoma. In particular embodiments, the antigen is CD19. In particular embodiments, the neoplasia is ovarian cancer, the antigen is MUC16.

The presently disclosed subject matter also provides an immunoresponsive cell (e.g., a T cell, Tumor Infiltrating Lymphocyte, Natural Killer (NK) cell, cytotoxic T lymphocyte (CTL), Natural Killer T (NKT) cells or regulatory T cell), comprising (a) an antigen binding receptor (e.g., CAR or TCR) directed toward a target antigen of interest, and (b) a modified promoter/enhancer at an endogenous (native) IL-18 gene locus. In certain non-limiting embodiments, the modified promoter/enhancer increases IL-18 gene expression. In certain non-limiting embodiments, a constitutive promoter is placed to drive endogenous IL-18 gene expression. In certain non-limiting embodiments, the constitutive promoter is selected from the group consisting of a CMV promoter, a EF1a promoter, a SV40 promoter, a PGK1 promoter, a Ubc promoter, a beta-actin promoter, and a CAG promoter. Accordingly, the presently disclosed subjection matter further provides methods of using such immunoresponsive cells for treating and/or preventing a neoplasia (e.g., cancer), infectious disease, or other pathologies that would benefit from an augmented immune response.

### BRIEF DESCRIPTION OF THE FIGURES

The following Detailed Description, given by way of example, but not intended to limit the invention to specific embodiments described, may be understood in conjunction with the accompanying drawings.

FIGS. 1A-1D show representations of the human CAR constructs. A) Human IL-18 secreting CAR constructs. B) Human CAR constructs of 1928z, 1928z-hIL18, 4H1128z and 4H1128z-hIL18, where the antigen recognizing domains are anti-hCD19 scFv or anti-Muc16^{ecto} scFv. C) and D) Retroviral vectors of human IL-18 secreting CAR.

FIG. 2 shows a representation of the human CAR constructs: 19BBz-hIL18

FIGS. 3A-3D show in vitro cytokine secretion of IL-18, IFNγ and IL-2 after coculture with NALM6 tumor cells (E:T 1:1). A) IL18 secretion. B) IFNγ secretion. C) IL-2 secretion. D) Data in scatter plots. Representative data from one of two experiments.

FIGS. 4A-4D A) and C) show in vitro proliferation assay with 1928z and 1928z-hIL18 CAR T cells when co-cultured with NALM6 tumor cells (E:T 1:1). CAR T cells were stimulated on day 0 and day 7. B) and D) show in vitro cytotoxicity utilizing standard 4 h ⁵¹Cr release assay comparing 1928z and 1928z-hIL18 CAR T cells.

FIGS. 5A-5D. A) and B) show survival curve comparing 1928z and 1928z-hIL18 CAR T cells in a NALM6-GFP⁺/Luc⁺-tumor bearing xenograft Seid-Beige mouse model. Representative data of one out of two experiments. C and D) show bioluminescence imaging comparing 1928z and 1928z-hIL18 CAR T cells in Scid-Beige mice inoculated with NALM6-GFP⁺/Luc⁺ tumor cells.

FIGS. 6A-6C show representations of murine CAR T cell constructs. A) Murine IL-18 secreting CAR constructs. B) Murine CAR constructs of: 19m28mz, 19m28mz-mIL18, 4H11m28mz and 4H11m28mz-mIL18. C) Retroviral vector of murine IL-18 secreting CAR.

FIGS. 7A-7B shows in vitro murine IL-18 secretion after co-stimulation with EL4hCD19 tumor cells (E:T 1:1).

FIGS. 8A-8D. A) and D) survival curve of EL4hCD19⁺ tumor-bearing mice treated with full dose (2.5×10⁶ CAR T cells mouse). B) IL-18 secreting first-generation CAR T cells (19mz-mIL18) also demonstrated enhanced anti-tumor effect and significantly increased mice long-term survival, compared to 19m28mz CAR T cells and controls. C) Survival curve of mice treated with 19m28mz-mIL18 CAR T cells on a delayed tumor model. Mice were inoculated with EL4hCD19⁺ tumor cells on day 0 and treated with CAR T cells on day 7 after tumor inoculation.

FIG. 9 shows survival curves of mice inoculated with EL4hCD19⁺ tumor cells on day 0, treated with 19m28mz-mIL18 CAR T cells on day 1 and re-challenged with EL4hCD19⁺ tumor cells on day 40. Control untreated mice were inoculated with EL4hCD19⁺ tumor cells either on day 0 or on day 40.

FIGS. 10A-10E. A), C) and E) show CAR T cell peripheral blood in vivo expansion quantified by flow cytometry analysis, with correspondent dot plot (bottom). Results are pooled from two independent experiments. B) and D) show peripheral blood B cell aplasia quantified by flow cytometry analysis, with correspondent dot plot (bottom).

FIG. 11 shows bone marrow PCR analysis of mice treated with 19m28mz-mIL18 CAR T cells. Material collected on days 35 (D35), 80 (D80), 120 (D120) and 150 (D150) after CAR T cell injection. Expected size of PCR product: 1450 bp. 19m28mz-mIL18 CAR T cell DNA was used as positive control and water was used as negative control.

FIG. 12A-12D show serum cytokine quantification on day 7, comparing 19m28mz and 19m28mz-mIL18 CAR T cells. Analysis was performed for A) IFNγ, B) TNFα, C) IL-6 and D) IL-18, IFNγ TNFα and IL-6.

FIG. 13 shows that mass cytometry was utilized to analyze day 18 bone marrow samples from mice treated with 19m28mz or 19m28mz-mIL18 CAR T cells. Log₂ fold change of manually gated populations comparing bone marrow of mice treated with 19m28mz and 19m28mz-mIL18 CAR T cells

FIG. 14 shows the percentage of endogenous CD8 non-CAR T cells and CD8 19m28mz-mIL18 CAR T cells in the bone marrow of mice treated with 19m28mz-mIL18 CAR T cells. Phenotypic analysis was performed with bone marrow endogenous cells of mice treated with 19m28mz or 19m28mz-mIL18 CAR T cells. The endogenous cells include: endogenous CD8 T cells, macrophages, and dendritic cells.

FIG. 15 shows survival curves of mice inoculated with both EL4hCD19⁺ and EL4hCD19⁻ tumor cells and treated with 19m28mz or 19m28mz-mIL18 CAR T cells.

FIG. 16 shows Elispot-IFNγ images comparing 19m28mz, 19m28mz-mIL18 CAR T cells and naive mice CAR-negative splenocytes after 24 hours exposure to EL4hCD19⁺ tumor cells. Representative data of one experiment out of 2 independent experiments. Elispot-IFNγ results comparing CAR-negative splenocytes from mice treated with 19m28mz, 19m28mz-mIL18 CAR T cells or naive mice when exposed to EL4hCD19⁺ tumor cells. Spots counted per 1×10⁵ splenocytes (1:1 E:T ratio) after 24 hours of exposure

FIG. 17 shows IFNγ cytokine quantification comparing CAR-negative splenocytes from mice treated with 19m28mz, 19m28mz-mIL18 CAR T cells or naive mice after 24 hours of exposure to EL4hCD19⁺ tumor cells (1:1 E:T ratio). IFNγ cytokine quantification comparing CAR-negative splenocytes from mice treated with 19m28mz, 19m28mz-mIL18 CAR T cells or naive mice after 24 hours of exposure to EL4hCD19⁻ tumor cells (1:1 E:T ratio). Results are pooled data of two independent experiments.

FIG. 18 shows survival curves of mice treated with 19m28mz-mIL18 CAR T cells with macrophage depletion (Clodronate Liposome) or without macrophage depletion (PBS Liposome).

FIGS. 19A-19B show anti-Muc16^{ecto} 19m28mz-mIL18 CAR T cells enhance survival of ovarian tumor-bearing syngeneic mice. C57BL/6 mice were inoculated with ID8 tumor cells I.P. on day 0 and treated with 2×10⁶ anti-Muc16^{ecto} 4H11m28mz or 4H11m28mz-mIL18 CAR T cells either on day 24 (A) or day 42 (B). 19m28mz-mIL18 CAR T cells were capable of significantly enhancing survival in both early (p=0.002) and delayed tumor models (p=0.0008).

FIG. 20 shows images describing the vectors for generating the pmel TCR and pmel TCR/IL-18 T cells

FIG. 21 shows in vitro IL-18 secretion of lmel TCR/IL18 T cells after 24 hs coculture with tumor cells

FIG. 22 shows the in vitro cytotoxic potential of armored IL-18 pmel-1 TCR T cells on B16F10 tumor cells that were transduced to express a GFP/luciferase gene was then analyzed and compared to the mCD19t control pmel-1 T cells. The modified pmel-1 T cells were co-cultured at various Effector to Target ratios with the gp100 positive B16F10 melanoma tumor cells for 24 hours. After 24 hours, the luciferin substrate was added and the luminescence of the cells was measured and used to calculate percent lysis of the tumor cells by the T cells.

FIG. 23 shows survival curves and the comparison of tumor volumes between the experimental groups.

### DETAILED DESCRIPTION OF THE INVENTION

The presently disclosed subject matter provides cells, including genetically modified immunoresponsive cells (e.g., T cells, Natural Killer (NK) cells, cytotoxic T lymphocytes (CTL) cells) comprising a combination of an antigen-recognizing receptor (e.g., TCR or CAR) and a secretable IL-18 polypeptide (e.g., an exogenous IL-18 polypeptide). The presently disclosed also provides methods of using such cells for treating and/or preventing a neoplasia or other pathologies where an increase in an antigen-specific immune response is desired. The presently disclosed subject is based, at least in part, on the discovery that a secretable IL-18 polypeptide enhances the anti-tumor effect of an immunoresponsive cell (e.g., a CAR T cell or a TCR T cell). In particular, the co-expression of IL-18 polypeptide and 1928z CAR was observed to increase proliferation, cytokine secretion and cell persistence of an activated immuno-reactive cell in the tumor microenvironment.

Malignant cells have developed a series of mechanisms to protect themselves from immune recognition and elimination. The presently disclosed subject provides immunogenicity within the tumor microenvironment for tumor eradication, and represents a significant advance over conventional adoptive T cell therapy. In certain non-limiting embodiments, it provides an option of foregoing some or all ancillary treatments such as prior conditioning of the host with total body irradiation, high-dose chemotherapy, and/or post-infusion cytokine support.

### 1. Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

By “activates an immunoresponsive cell” is meant induction of signal transduction or changes in protein expression in the cell resulting in initiation of an immune response. For example, when CD3 Chains cluster in response to ligand binding and immunoreceptor tyrosine-based inhibition motifs (ITAMs) a signal transduction cascade is produced. In certain embodiments, when an endogenous TCR or an exogenous CAR binds antigen, a formation of an immunological synapse occurs that includes clustering of many molecules near the bound receptor (e.g. CD4 or CD8, CD3γ/δ/ε/ζ, etc.) This clustering of membrane bound signaling molecules allows for ITAM motifs contained within the CD3 chains to become phosphorylated. This phosphorylation in turn initiates a T cell activation pathway ultimately activating transcription factors, such as NF-κB and AP-1. These transcription factors induce global gene expression of the T cell to increase IL-2 production for proliferation and expression of master regulator T cell proteins in order to initiate a T cell mediated immune response. By “stimulates an immunoresponsive cell” is meant a signal that results in a robust and sustained immune response. In various embodiments, this occurs after immune cell (e.g., T-cell) activation or concomitantly mediated through receptors including, but not limited to, CD28, CD137 (4-1BB), OX40, CD40 and ICOS. Without being bound to a particular theory, receiving multiple stimulatory signals is important to mount a robust and long-term T cell mediated immune response. Without receiving these stimulatory signals, T cells quickly become inhibited and unresponsive to antigen. While the effects of these co-stimulatory signals vary and remain partially understood, they generally result in increasing gene expression in order to generate long lived, proliferative, and anti-apoptotic T cells that robustly respond to antigen for complete and sustained eradication.

The term “antigen recognizing receptor” as used herein refers to a receptor that is capable of activating an immune cell (e.g., a T-cell) in response to antigen binding. Exemplary antigen recognizing receptors may be native or endogenous T cell receptors or chimerie antigen receptors in which an antigen-binding domain is fused to an intracellular signaling domain capable of activating an immune cell (e.g., a T-cell).

As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments that lack the Fe fragment of intact antibody, clear more rapidly from the circulation, and may have less non-specific tissue binding of an intact antibody (Wahl et al., J. Nucl. Med. 24:316-325 (1983). The antibodies of the invention comprise whole native antibodies, bispecific antibodies; chimeric antibodies; Fab, Fab′, single chain V region fragments (scFv), fusion polypeptides, and unconventional antibodies.

As used herein, the term “single-chain variable fragment” or “scFv” is a fusion protein of the variable regions of the heavy (VH) and light chains (VL) of an immunoglobulin covalently linked to form a VH:VL heterodimer. The heavy (VH) and light chains (VL) are either joined directly or joined by a peptide-encoding linker (e.g., 10, 15, 20, 25 amino acids), which connects the N-terminus of the VH with the C-terminus of the VL, or the C-terminus of the VH with the N-terminus of the VL. The linker is usually rich in glycine for flexibility, as well as serine or threonine for solubility. Despite removal of the constant regions and the introduction of a linker, scFv proteins retain the specificity of the original immunoglobulin. Single chain Fv polypeptide antibodies can be expressed from a nucleic acid including VH- and VL-encoding sequences as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hyrbidoma (Larchmt) 2008 27(6):455-51; Peter et al., J Cachexia Sarcopenia Muscle 2012 Aug. 12; Shieh et al., J Imunol 2009 183(4):2277-85; Giomarelli et al., Thromb Haemost 2007 97(6):955-63; Fife eta., J Clin Invst 2006 116(8): 2252-61; Brocks et al., Immunotechnology 1997 3(3): 173-84; Moosmayer et al., Ther Immunol 1995 2(10:31-40). Agonistic scFvs having stimulatory activity have been described (see, e.g., Peter et al., J Biol Chem 2003 25278(38):36740-7; Xie et al., Nat Biotech 1997 15(8): 768-71; Ledbetter et al., Crit Rev Immunol 1997 17(5-6):427-55; Ho et al., BioChim Biophys Acta 2003 1638(3):257-66).

As used herein, the term “affinity” is meant a measure of binding strength. Without being bound to theory, affinity depends on the closeness of stereochemical fit between antibody combining sites and antigen determinants, on the size of the area of contact between them, and on the distribution of charged and hydrophobic groups. Affinity also includes the term “avidity,” which refers to the strength of the antigen-antibody bond after formation of reversible complexes. Methods for calculating the affinity of an antibody for an antigen are known in the art, including use of binding experiments to calculate affinity. Antibody activity in functional assays (e.g., flow cytometry assay) is also reflective of antibody affinity. Antibodies and affinities can be phenotypically characterized and compared using functional assays (e.g., flow cytometry assay).

The term “chimeric antigen receptor” or “CAR” as used herein refers to an antigen-binding domain that is fused to an intracellular signaling domain capable of activating or stimulating an immune cell, and in certain embodiments, the CAR also comprises a transmembrane domain. In certain embodiments the CAR's extracellular antigen-binding domain is composed of a single chain variable fragment (scFv) derived from fusing the variable heavy and light regions of a murine or humanized monoclonal antibody. Alternatively, scFvs may be used that are derived from Fab's (instead of from an antibody, e.g., obtained from Fab libraries). In various embodiments, the scFv is fused to the transmembrane domain and then to the intracellular signaling domain. “First-generation” CARs include those that solely provide CD3ζ signals upon antigen binding, “Second-generation” CARs include those that provide both co-stimulation (e.g., CD28 or CD137) and activation (CD3ζ). “Third-generation” CARs include those that provide multiple co-stimulation (e.g. CD28 and CD137) and activation (CD3ζ). In various embodiments, the CAR is selected to have high affinity or avidity for the antigen.

The term “immunosuppressive activity” is meant induction of signal transduction or changes in protein expression in a cell (e.g., an activated immunoresponsive cell) resulting in a decrease in an immune response. Polypeptides known to suppress or decrease an immune response via their binding include CD47, PD-1, CTLA-4, and their corresponding ligands, including SIRPa, PD-L1, PD-L2, B7-1, and B7-2. Such polypeptides are present in the tumor microenvironment and inhibit immune responses to neoplastic cells. In various embodiments, inhibiting, blocking, or antagonizing the interaction of immunosuppressive polypeptides and/or their ligands enhances the immune response of the immunoresponsive cell.

The term “immunostimulatory activity” is meant induction of signal transduction or changes in protein expression in a cell (e.g., an activated immunoresponsive cell) resulting in an increase in an immune response. Immunostimulatory activity may include pro-inflammatory activity. Polypeptides known to stimulate or increase an immune response via their binding include CD28, OX-40, 4-1BB, and their corresponding ligands, including B7-1, B7-2, OX-40L, and 4-1BBL. Such polypeptides are present in the tumor microenvironment and activate immune responses to neoplastic cells. In various embodiments, promoting, stimulating, or agonizing pro-inflammatory polypeptides and/or their ligands enhances the immune response of the immunoresponsive cell.

Nucleic acid molecules useful in the methods of the invention include any nucleic acid molecule that encodes a polypeptide of the invention or a fragment thereof. Such nucleic acid molecules need not be 100% homologous or identical with an endogenous nucleic acid sequence, but will typically exhibit substantial identity. Polynucleotides having “substantial identity” or “substantial homology” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In certain embodiments, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Rogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” or “substantially homologous” is meant a polypeptide or nucleic acid molecule exhibiting at least about 50% homologous or identical to a reference amino acid sequence (for example, any one of the amino acid sequences described herein) or nucleic acid sequence (for example, any one of the nucleic acid sequences described herein). Preferably, such a sequence is at least about 60%, about 80%, about 85%, about 90%, about 95%, about 99%, or about 100% homologous or identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

By “analog” is meant a structurally related polypeptide or nucleic acid molecule having the function of a reference polypeptide or nucleic acid molecule.

The term “ligand” as used herein refers to a molecule that binds to a receptor. In particular, the ligand binds a receptor on another cell, allowing for cell-to-cell recognition and/or interaction.

The term “constitutive expression” as used herein refers to expression under all physiological conditions.

By “disease” is meant any condition or disorder that damages or interferes with the normal function of a cell, tissue, or organ. Examples of diseases include neoplasia or pathogen infection of cell.

By “effective amount” is meant an amount sufficient to have a therapeutic effect. In one embodiment, an “effective amount” is an amount sufficient to arrest, ameliorate, or inhibit the continued proliferation, growth, or metastasis (e.g., invasion, or migration) of a neoplasia.

By “endogenous” is meant a nucleic acid molecule or polypeptide that is normally expressed in a cell or tissue.

By “enforcing tolerance” is meant preventing the activity of self-reactive cells or immunoresponsive cells that target transplanted organs or tissues.

By “exogenous” is meant a nucleic acid molecule or polypeptide that is not endogenously present in the cell. The term “exogenous” would therefore encompass any recombinant nucleic acid molecule or polypeptide expressed in a cell, such as foreign, heterologous, and over-expressed nucleic acid molecules and polypeptides. By “exogenous” nucleic acid is meant a nucleic acid not present in a native wild type cell; for example an exogenous nucleic acid may vary from an endogenous counterpart by sequence, by position/location, or both. For clarity, an exogenous nucleic acid may have the same or different sequence relative to its native endogenous counterpart; it may be introduced by genetic engineering into the cell itself or a progenitor thereof, and may optionally be linked to alternative control sequences, such as a non-native promoter or secretory sequence.

By a “heterologous nucleic acid molecule or polypeptide” is meant a nucleic acid molecule (e.g., a cDNA, DNA or RNA molecule) or polypeptide that is not normally present in a cell or sample obtained from a cell. This nucleic acid may be from another organism, or it may be, for example, an mRNA molecule that is not normally expressed in a cell or sample.

By “immunoresponsive cell” is meant a cell that functions in an immune response or a progenitor, or progeny thereof.

By “increase” is meant to alter positively by at least about 5%. An alteration may be by about 5%, about 10%, about 25%, about 30%, about 50%, about 75%, about 100% or more.

By “isolated cell” is meant a cell that is separated from the molecular and/or cellular components that naturally accompany the cell.

The terms “isolated,” “purified,” or “biologically pure” refer to material that is free to varying degrees From components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

The term “tumor antigen-binding domain” as used herein refers to a domain capable of specifically binding a particular antigenic determinant or set of antigenic determinants present on a tumor.

The term “obtaining” as in “obtaining the agent” is intended to include purchasing, synthesizing or otherwise acquiring the agent (or indicated substance or material).

“Linker”, as used herein, shall mean a functional group (e.g., chemical or polypeptide) that covalently attaches two or more polypeptides or nucleic acids so that they are connected to one another. As used herein, a “peptide linker” refers to one or more amino acids used to couple two proteins together (e.g., to couple V_{H} and V_{L} domains). An exemplary linker sequence used in the invention is GGGGSGGGGSGGGGS [SEQ ID NO: 23].

By “modulate” is meant positively or negatively alter. Exemplary modulations include a about 1%, about 2%, about 5%, about 10%, about 25%, about 50%, about 75%, or about 100% change.

By “neoplasia” is meant a disease characterized by the pathological proliferation of a cell or tissue and its subsequent migration to or invasion of other tissues or organs. Neoplasia growth is typically uncontrolled and progressive, and occurs under conditions that would not elicit, or would cause cessation of, multiplication of normal cells. Neoplasias can affect a variety of cell types, tissues, or organs, including but not limited to an organ selected from the group consisting of bladder, bone, brain, breast, cartilage, glia, esophagus, fallopian tube, gallbladder, heart, intestines, kidney, liver, lung, lymph node, nervous tissue, ovaries, pancreas, prostate, skeletal muscle, skin, spinal cord. spleen, stomach, testes, thymus, thyroid, trachea, urogenital tract, ureter, urethra, uterus, and vagina, or a tissue or cell type thereof. Neoplasias include cancers, such as sarcomas, carcinomas, or plasmacytomas (malignant tumor of the plasma cells).

By “receptor” is meant a polypeptide, or portion thereof, present on a cell membrane that selectively binds one or more ligand.

By “reduce” is meant to alter negatively by at least about 5%. An alteration may be by about 5%, about 10%, about 25%, about 30%, about 50%, about 75%, or even by about 100%.

By “recognize” is meant selectively binds a target. AT cell that recognizes a virus typically expresses a receptor that binds an antigen expressed by the virus.

By “reference” or “control” is meant a standard of comparison. For example, the level of scFv-antigen binding by a cell expressing a CAR and an scFv may be compared to the level of scFv-antigen binding in a corresponding cell expressing CAR alone.

By “secreted” is meant a polypeptide that is released from a cell via the secretory pathway through the endoplasmic reticulum, Golgi apparatus, and as a vesicle that transiently fuses at the cell plasma membrane, releasing the proteins outside of the cell.

By “signal sequence” or “leader sequence” is meant a peptide sequence (e.g., 5, 10, 15, 20, 25 or 30 amino acids) present at the N-terminus of newly synthesized proteins that directs their entry to the secretory pathway. Exemplary leader sequences include, but is not limited to, the IL-2 signal sequence: MYRMQLLSCIALSLALVTNS [SEQ NO: ID 8] (human), MYSMQLASCVTLTLVLLVNS [SEQ ID NO: 24] (mouse); the kappa leader sequence: METPAQLLFLLLLWLPDTTG [SEQ ID NO: 25] (human), METDTLLLWVLLLWVPGSTG [SEQ ID NO: 26] (mouse); the CD8 leader sequence: MALPVTALLLPLALLLHAARP [SEQ ID NO: 27] (human); the albumin signal sequence: MKWVTFISLLFSSAYS [SEQ ID NO: 28] (human); and the prolactin signal sequence: MDSKGSSQKGSRLLLLLVVSNLLLCQGVVS [SEQ ID NO: 29] (human).

By “soluble” is meant a polypeptide that is freely diffusible in an aqueous environment (e.g., not membrane bound).

By “specifically binds” is meant a polypeptide or fragment thereof that recognizes and binds a biological molecule of interest (e.g., a polypeptide), but which does not substantially recognize and bind other molecules in a sample, for example, a biological sample, which naturally includes a polypeptide of the invention.

The term “tumor antigen” as used herein refers to an antigen (e.g., a polypeptide) that is uniquely or differentially expressed on a tumor cell compared to a normal or non-IS neoplastic cell. In certain embodiments, a tumor antigen includes any polypeptide expressed by a tumor that is capable of activating or inducing an immune response via an antigen recognizing receptor (e.g., CD19, MUC-16) or capable of suppressing an immune response via receptor-ligand binding (e.g., CD47, PD-L1/L2, B7.1/2).

The terms “comprises”, “comprising”, and are intended to have the broad meaning ascribed to them in U.S. Patent Law and can mean “includes”, “including” and the like.

As used herein, “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

The term “subject” as used herein refers to a vertebrate, preferably a mammal, more preferably a human.

The term “immunocompromised” as used herein refers to a subject who has an immunodeficiency. The subject is very vulnerable to opportunistic infections, infections caused by organisms that usually do not cause disease in a person with a healthy immune system, but can affect people with a poorly functioning or suppressed immune system.

Other aspects of the invention are described in the following disclosure and are within the ambit of the invention.

### 2. Antigen Recognizing Receptors

The present disclosure provides antigen recognizing receptors that bind to an antigen of interest. In certain embodiments, the antigen recognizing receptor is a chimeric antigen receptor (CAR). In certain embodiments, the antigen recognizing receptor is a T-cell receptor (TCR). The antigen recognizing receptor can bind to a tumor antigen or a pathogen antigen.

### 2.1. Antigens

In certain embodiments, the antigen recognizing receptor binds to a tumor antigen. Any suitable tumor antigen (antigenic peptide) is suitable for use in the tumor-related embodiments described herein. Sources of antigen include, but are not limited to cancer proteins. The antigen can be expressed as a peptide or as an intact protein or portion thereof. The intact protein or a portion thereof can be native or mutagenized. Non-limiting examples of tumor antigens include carbonic anhydrase IX (CA1X), carcinoembryonic antigen (CEA), CD8, CD7, CDI0, CD19, CD20, CD22, CD30, CD33, CLL1, CD34, CD38, CD41, CD44, CD49f, CD56, CD74, CD133, CD138, CD123, CD44V6, an antigen of a cytomegalovirus (CMV) infected cell (e.g., a cell surface antigen), epithelial glycoprotein-2 (EGP-2), epithelial glycoprotein-40 (EGP-40), epithelial cell adhesion molecule (EpCAM), receptor tyrosine-protein kinases erb-B2,3,4 (erb-B2,3,4), folate-binding protein (FBP), fetal acetylcholine receptor (AChR), folate receptor-α, Ganglioside G2 (GD2), Ganglioside G3 (GD3), human Epidermal Growth Factor Receptor 2 (HER-2), human telomerase reverse transcriptase (hTERT), Interleukin-13 receptor subunit alpha-2 (IL-13Rα2), κ-light chain, kinase insert domain receptor (KDR), Lewis Y (LeY), L1 cell adhesion molecule (L1CAM), melanoma antigen family A, 1 (MAGE-A1), Mucin 16 (MUC16), Mucin 1 (MUC1), Mesothelin (MSLN), ERBB2, MAGEA3, p53, MART1, GP100, Proteinase3 (PR1), Tyrosinase, Survivin, hTERT, EphA2, NKG2D ligands, cancer-testis antigen NY-ES0-1, oncofetal antigen (hST4), prostate stem cell antigen (PSCA), prostate-specific membrane antigen (PSMA). ROR1, tumor-associated glycoprotein 72 (TAG-72), vascular endothelial growth factor R2 (VEGF-R2), and Wilms tumor protein (WT-1), BCMA, NKCS1, EGF1R, EGFR-VIII, and ERBB.

In certain embodiments, the antigen recognizing receptor binds to a pathogen antigen, e.g., for use in treating and/or preventing a pathogen infection or other infectious disease, for example, in an immunocompromised subject. In certain embodiments, pathogen includes a virus, bacteria, fungi, parasite or protozoa capable of causing disease.

Non-limiting examples of viruses include, Retroviridae (e.g. human immunodeficiency viruses, such as HIV-1 (also referred to as HDTV-III, LAVE or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviridae (e.g. vesicular stomatitis viruses, rabies viruses); Filoviridae (e.g. ebola viruses): Paramyxoviridae (e.g. parainfluenza viruses, mumps virus, measles virus, respiratory syncytial virus); Orthomyxoviridae(e.g. influenza viruses); Bungaviridae (e.g. Hantaan viruses, bunga viruses, phleboviruses and Naira viruses); Arena viridae (hemorrhagic fever viruses); Reoviridae (e.g. reovinises, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g. African swine fever virus); and unclassified viruses (e.g. the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

Non-limiting examples of bacteria include Pasteurella, Staphylococci, Streptococcus, Escherichia coli, Pseudomonas species, and Salmonella species. Specific examples of infectious bacteria include but are not limited to, Helicobacter pyloris, Borelia burgdorferi, Legionella pneumophilia, Mycobacteria sps (e.g. M. tuberculosis. M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus antracis, Corynebacterium diphtheriae, corynebacterium sp., Erysipelothrix rhusiopathiae, Clostridium perfringers, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, Rickettsia, and Actinomyces israelli.

In certain embodiments, the pathogen antigen is a viral antigen present in Cytomegalovirus (CMV), a viral antigen present in Epstein Barr Virus (EBV), a viral antigen present in Human Immunodeficiency Virus (HIV), or a viral antigen present in influenza virus.

### 2.2. T-Cell Receptor (TCR)

In certain embodiments, the antigen recognizing receptor is a TCR. A TCR is a disulfide-linked heterodimeric protein consisting of two variable chains expressed as part of a complex with the invariant CD3 chain molecules. A TCR is found on the surface of T cells, and is responsible for recognizing antigens as peptides bound to major histocompatibility complex (MHC) molecules. In certain embodiments, a TCR comprises an alpha chain and a beta chain (encoded by TRA and TRB, respectively). In certain embodiments, a TCR comprises a gamma chain and a delta chain (encoded by TRG and TRD, respectively).

Each chain of a TCR is composed of two extracellular domains: Variable (V) region and a Constant (C) region. The Constant region is proximal to the cell membrane, followed by a transmembrane region and a short cytoplasmic tail. The Variable region binds to the peptide/MHC complex. The variable domain of both chains each has three complementarity determining regions (CDRs).

In certain embodiments, a TCR can form a receptor complex with three dimeric signaling modules CD3δ/ε,CD3γ/ε and CD247 ζ/ζ or ζ/η. When a TCR complex engages with its antigen and MHC (peptide/MHC), the T cell expressing the TCR complex is activated.

In certain embodiments, the presently disclosed subject matter provides a recombinant TCR. In certain embodiments, the TOR is a non-naturally occurring TCR. In certain embodiments, the TCR differs from any naturally occurring TCR by at least one amino acid residue. In certain embodiments, the TCR differs from any naturally occurring TCR by at least 2, 3, 4, 5, 6, 7, 8, 9, 10, 11, 12, 13, 14, 15, 20, 25, 30, 40, 50, 60, 70. 80, 90, 100 or more amino acid residues. In certain embodiments, the TCR is modified from a naturally occurring TCR by at least one amino acid residue. In certain embodiments, the TCR is modified from a naturally occurring TCR by at least 2, 3, 4, 5, 6, 7, 8, 9, 10, 11, 12, 13, 14, 15, 20, 25, 30, 40, 50, 60, 70, 80, 90, 100 or more amino acid residues.

### 2.3. Chimeric Antigen Receptor (CAR)

In certain embodiments, the antigen recognizing receptor is a CAR. CARs are engineered receptors, which graft or confer a specificity of interest onto an immune effector cell. CARs can be used to graft the specificity of a monoclonal antibody onto a T cell; with transfer of their coding sequence facilitated by retroviral vectors.

There are three generations of CARs. “First generation” CARs are typically composed of an extracellular antigen binding domain (e.g., a single-chain variable fragments (scFv)) fused to a transmembrane domain, fused to cytoplasmic/intracellular signaling domain of the T cell receptor chain. “First generation” CARs typically have the intracellular signaling domain from the CD3ζ-chain, which is the primary transmitter of signals from endogenous TCRs. “First generation” CARs can provide de novo antigen recognition and cause activation of both CD4⁺ and CD8⁺ T cells through their CD3ζ chain signaling domain in a single fusion molecule, independent of HLA-mediated antigen presentation. “Second generation” CARs add intracellular signaling domains from various co-stimulatory molecules (e.g., CD28, 4-1BB, ICOS, OX40) to the cytoplasmic tail of the CAR to provide additional signals to the T cell. “Second generation” CARs comprise those that provide both co-stimulation (e.g., CD28 or 4-1BB) and activation (CD3ζ). Preclinical studies have indicated that “Second Generation” CARs can improve the anti-tumor activity of T cells. For example, robust efficacy of “Second Generation” CAR modified T cells was demonstrated in clinical trials targeting the CD19 molecule in patients with chronic lymphoblastic leukemia (CLL) and acute lymphoblastic leukemia (ALL). “Third generation” CARs comprise those that provide multiple co-stimulation (e.g., CD28 and 4-1BB) and activation (CD3ζ).

In certain non-limiting embodiments, the extracellular antigen-binding domain of the CAR (embodied, for example, an scFv or an analog thereof) binds to an antigen with a dissociation constant (K_{d}) of about 2×10⁻⁷ M or less. In certain embodiments, the K_{d} is about 2×10⁻⁷ M or less, about 1×10⁻⁷ M or less, about 9×10⁻⁸ M or less, about 1×10⁻⁸ M or less, about 9×10⁻⁹ M or less, about 5×10⁻⁹ M or less, about 4×10⁻⁹ M or less, about 3×10⁻⁹ or less, about 2×10⁻⁹ M or less, or about 1×10⁻⁹ M or less. In certain non-limiting embodiments, the K_{d} is about 3×10⁻⁹ M or less. In certain non-limiting embodiments, the K_{d} is from about 1×10⁻⁹ M to about 3×10⁻⁷ M. In certain non-limiting embodiments, the K_{d} is from about 1.5×10⁻⁹ M to about 3×10⁻⁷ M. In certain non-limiting embodiments, the K_{d} is from about 1.5×10⁻⁹ M to about 2.7×10⁻⁷ M.

Binding of the extracellular antigen-binding domain (for example, in an scFv or an analog thereof) of a presently disclosed antigen-targeted CAR can be confirmed by, for example, enzyme-linked immunosorbent assay (ELISA), radioimmunoassay (RIA), FACS analysis, bioassay (e.g., growth inhibition), or Western Blot assay. Each of these assays generally detect the presence of protein-antibody complexes of particular interest by employing a labeled reagent (e.g., an antibody, or an scFv) specific for the complex of interest. For example, the scFv can be radioactively labeled and used in a radioimmunoassay (RIA) (see, for example, Weintraub, B., Principles of Radioimmunoassays, Seventh Training Course on Radioligand Assay Techniques, The Endocrine Society, March 1986, which is incorporated by reference herein). The radioactive isotope can be detected by such means as the use of a y counter or a scintillation counter or by autoradiography. In certain embodiments, the extracellular antigen-binding domain of the CAR is labeled with a fluorescent marker. Non-limiting examples of fluorescent markers include green fluorescent protein (GFP), blue fluorescent protein (e.g., EBFP, EBFP2, Azurite, and mKalama1), cyan fluorescent protein (e.g., ECFP, Cerulean, and CyPet), and yellow fluorescent protein (e.g., YFP, Citrine, Venus, and YPet).

In accordance with the presently disclosed subject matter, a CARs can comprise an extracellular antigen-binding domain, a transmembrane domain and an intracellular signaling domain, wherein the extracellular antigen-binding domain specifically binds to an antigen, e.g., a tumor antigen or a pathogen antigen.

### 2.3.1. Extracellular Antigen-Binding Domain of A CAR

In certain embodiments, the extracellular antigen-binding domain specifically binds to an antigen. In certain embodiments, the extracellular antigen-binding domain is an scFv. In certain embodiments, the scFv is a human scFv. In certain embodiments, the scfv is a humanized scFv. In certain embodiments, the extracellular antigen-binding domain is a Fab, which is optionally crosslinked. In certain embodiments, the extracellular binding domain is a F(ab)₂. In certain embodiments, any of the foregoing molecules may be comprised in a fusion protein with a heterologous sequence to form the extracellular antigen-binding domain. In certain embodiments, the scFv is identified by screening scFv phage library with an antigen-Fc fusion protein. In certain embodiments, the antigen is a tumor antigen. In certain embodiments, the antigen is a pathogen antigen.

### 2.3.2. Transmembrane Domain of a CAR

In certain non-limiting embodiments, the transmembrane domain of the CAR comprises a hydrophobic alpha helix that spans at least a portion of the membrane. Different transmembrane domains result in different receptor stability. After antigen recognition, receptors cluster and a signal is transmitted to the cell. In accordance with the presently disclosed subject matter, the transmembrane domain of the CAR can comprise a CD8 polypeptide, a CD28 polypeptide, a CD3ζ polypeptide, a CD4 polypeptide, a 4-1BB polypeptide, an OX40 polypeptide, an ICOS polypeptide, a synthetic peptide (not based on a protein associated with the immune response), or a combination thereof.

In certain embodiments, the transmembrane domain comprises a CD8 polypeptide. In certain embodiments, the CD8 polypeptide has an amino acid sequence that is at least about 85%, about 90%, about 95%, about 96%, about 97%, about 98%, about 99% or about 100% homologous to the sequence having a NCBI Reference No: NP_001139345.1 (SEQ ID NO: 9) (homology herein may be determined using standard software such as BLAST or FASTA) as provided below, or fragments thereof, and/or may optionally comprise up to one or up to two or up to three conservative amino acid substitutions. In certain embodiments, the CD8 polypeptide can have an amino acid sequence that is a consecutive portion of SEQ ID NO: 9 which is at least 20, or at least 30, or at least 40, or at least 50, and up to 235 amino acids in length. Alternatively or additionally, in non-limiting various embodiments, the CD8 polypeptide comprises or has an amino acid sequence of amino acids 1 to 235, 1, to 50, 50 to 100, 100 to 150, 150 to 200, or 200 to 235 of SEQ ID NO: 9. In certain embodiments, the CAR of the presently disclosed comprises a transmembrane domain comprising a CD8 polypeptide that comprises an amino acid sequence of amino acids 137 to 209 of SEQ ID NO: 9.

[SEQ ID NO: 9]
MALPVTALLLPLALLLHAARPSQFRVSPLDRTWNLGETVELKCQVLLSN
PTSGCSWLFQPRGAAASPTFLLYLSQNKPKAAEGLDTQRFSGKRLGDTF
VLTLSDFRRENEGYYFCSALSNSIMYFSHFVPVFLPAKPTTTPAPRPPT
PAPTIASQPLSLRPEACRPAAGGAVHTRGLDFACDIYIWAPLAGTCGVL
LLSLVITLYCNHRNRRRVCKCPRPVVKSGDKPSLSARYV

In certain embodiments, the CD8 polypeptide has an amino acid sequence that is at least about 85%, about 90%, about 95%, about 96%, about 97%, about 98%, about 99% or about 100% homologous to the sequence having a NCBI Reference No: AAA92533.1 (SEQ ID NO: 10) (homology herein may be determined using standard software such as BLAST or FASTA) as provided below, or fragments thereof, and/or may optionally comprise up to one or up to two or up to three conservative amino acid substitutions. In certain embodiments, the CD8 polypeptide can have an amino acid sequence that is a consecutive portion of SEQ ID NO: 10 which is at least about 20, or at least about 30, or at least about 40, or at least about 50, or at least about 60, or at least about 70, or at least about 100, or at least about 200, and up to 247 amino acids in length. Alternatively or additionally, in non-limiting various embodiments, the CD8 polypeptide comprises or has an amino acid sequence of amino acids 1 to 247, 1 to 50, 50 to 100, 100 to 150, 150 to 200, 151 to 219, or 200 to 247 of SEQ ID NO: 10. In certain embodiments, the CAR of the presently disclosed comprises a transmembrane domain comprising a CD8 polypeptide that comprises an amino acid sequence of amino acids 151 to 219 of SEQ ID NO: 10.

| [SEQ ID NO: 10] | |
|---|---|
| 1 | MASPLTRELS LNLLLMGESI ILGSGEAKPQ APELRIFPKK MDAELGQKVD LVCEVLGSVS |
| 61 | QGCSWLFQNS SSKLPQPTFV VYMASSHNKI TWDEKLNSSK LFSAVRDTNN KYVLTLNKFS |
| 121 | KENEGYYFCS VISNSVMYFS SVVPVLQKVN STTTKPVLRT PSPVHPTGTS QPQRPEDCRP |
| 181 | RGSVKGTGLD FACDIYIWAP LAGICVAPLL SLIITLICYH RSRKRVCKCP RPLVRQEGKP |
| 241 | RPSEKIV |

In certain embodiments, the CDS polypeptide comprises or has the amino acid sequence set forth in SEQ ID NO: 11, which is provided below:

[SEQ ID NO: 11]
STTTKPVLRTPSPVHPTGTSQPQRPEDCRPRGSVKGTGLDFACDIYIWAP
LAGICVALLLSLIITLICY

In accordance with the presently disclosed subject matter, a “CD8 nucleic acid molecule” refers to a polynucleotide encoding a CD8 polypeptide.

In certain embodiments, the CD8 nucleic acid molecule encoding the CD8 polypeptide having the amino acid sequence set forth in SEQ ID NO: 11 comprises nucleic acids having the sequence set forth in SEQ ID NO: 12 as provided below.

[SEQ ID NO: 12]
TCTACTACTACCAAGCCAGTGCTGCGAACTCCCTCACCTGTGCACCCTAC
CGGGACATCTCAGCCCCAGAGACCAGAAGATTGTCGGCCCCGTGGCTCAG
TGAAGGGGACCGGATTGGACTTCGCCTGTGATATTTACATCTGGGCACCC
TTGGCCGGAATCTGCGTGGCCCTTCTGCTGTCCTTGATCATCACTCTCAT
CTGCTAC

In certain embodiments, the transmembrane domain of a presently disclosed CAR comprises a CD28 polypeptide. The CD28 polypeptide can have an amino acid sequence that is at least about 85%, about 90%, about 95%, about 96%, about 97%, about 98%, about 99% or 100% homologous to the sequence having a NCBI Reference No: P10747 or NP_006130 (SEQ ID No: 2), or fragments thereof, and or may optionally comprise up to one or up to two or up to three conservative amino acid substitutions. In non-limiting certain embodiments, the CD28 polypeptide can have an amino acid sequence that is a consecutive portion of SEQ ID NO: 2 which is at least 20, or at least 30, or at least 40, or at least 50, and up to 220 amino acids in length. Alternatively or additionally, in non-limiting various embodiments, the CD28 polypeptide has an amino acid sequence of amino acids 1 to 220, 1 to 50, 50 to 100, 100 to 150, 114 to 220, 150 to 200, or 200 to 220 of SEQ ID NO: 2. In certain embodiments, the CD28 polypeptide comprised in the transmembrane domain of a presently disclosed CAR has an amino acid sequence of amino acids 153 to 179 of SEQ ID NO: 2.

SEQ ID NO: 2 is provided below:

| [SEQ ID NO: 2] | |
|---|---|
| 1 | MLRLLLALNL FPSIQVTGNK ILVKQSPMLV AYDNAVNLSC KYSYNLFSRE FRASLHKGLD |
| 61 | SAVEVCVVYG NYSQQLQVYS KTGFNCDGKL GNESVTFYLQ NLYVNQTDIY FCKIEVMYPP |
| 121 | PYLDNEKSNG TIIHVKGKHL CPSPLFPGPS KPFWVLVVVG GVLACYSLLV TVAFIIFWVR |
| 181 | SKRSRLLHSD YMNMTPRRPG PTRKHYQPYA PPRDFAAYRS |

In accordance with the presently disclosed subject matter, a “CD28 nucleic acid molecule” refers to a polynucleotide encoding a CD28 polypeptide. In certain embodiments, the CD28 nucleic acid molecule encoding the CD28 polypeptide having amino acids 153 to 179 of SEQ ID NO: 2 comprises nucleic acids having the sequence set forth in SEQ ID NO: 22 as provided below.

[SEQ ID NO: 22]
ttttgggtgctggtggtggttggtggagtcctggcttgctatagcttgct
agtaacagtggcctttattattttctgggtg

In certain non-limiting embodiments, a CAR can also comprise a spacer region that links the extracellular antigen-binding domain to the transmembrane domain. The spacer region can be flexible enough to allow the antigen binding domain to orient in different directions to facilitate antigen recognition. The spacer region can be the hinge region from IgG1, or the CH₂CH₃ region of immunoglobulin and portions of CD3, a portion of a CD28 polypeptide (e.g., a portion of SEQ ID NO: 2), a portion of a CD8 polypeptide (e.g., a portion of SEQ ID NO: 9, or a portion of SEQ ID NO: 10), a variation of any of the foregoing which is at least about 80%, at least about 85%, at least about 90%, or at least about 95% homologous thereto, or a synthetic spacer sequence.

### 2.3.3. Intracellular Signaling Domain of a CAR

In certain non-limiting embodiments, an intracellular signaling domain of the CAR can comprise a CD3ζ polypeptide, which can activate or stimulate a cell (e.g., a cell of the lymphoid lineage, e.g., a T cell). CD3ζ comprises 3 ITAMs, and transmits an activation signal to the cell (e.g., a cell of the lymphoid lineage, e.g., a T cell) after antigen is bound. In certain embodiments, the CD3ζ polypeptide has an amino acid sequence that is at least about 85%, about 90%, about 95%, about 96%, about 97%, about 98%, about 99% or about 100% homologous to the sequence having a NCBI Reference No: NP_932170 (SEQ ID No: 1), or fragments thereof, and/or may optionally comprise up to one or up to two or up to three conservative amino acid substitutions. In certain non-limiting embodiments, the CD3ζ polypeptide comprises or bas an amino acid sequence that is a consecutive portion of SEQ ID NO: 1, which is at least 20, or at least 30, or at least 40, or at least 50, and up to 164 amino acids in length. Alternatively or additionally, in non-limiting various embodiments, the CD3ζ polypeptide comprises or has an amino acid sequence of amino acids 1 to 164, 1 to 50, 50 to 100, 100 to 150, or 150 to 164 of SEQ ID NO: 1. In certain embodiments, the CD3ζ polypeptide comprises or has an amino acid sequence of amino acids 52 to 164 of SEQ ID NO: 1.

SEQ ID NO: 1 is provided below:

| [SEQ ID NO: 1] | |
|---|---|
| 1 | MKWKALFTAA ILQAQLPITE AQSFGLLDPK LCYLLDGILF IYGVILTALF LRVKFSRSAD |
| 61 | APAYQQGQNQ LYNELNLGRR EEYDVLDKRR GRDPEMGGKP QRRKNPQEGL YNELQKDKMA |
| 121 | EAYSEIGMKG ERRRGKGHDG LYQGLSTATK DTYDALHMQA LPPR |

In certain embodiments, the CD3ζ polypeptide has an amino acid sequence that is at least about 85%, about 90%, about 95%, about 96%, about 97%, about 98%, about 99% or about 100% homologous to the sequence having a NCBI Reference No: NP_001106864.2 (SEQ ID No: 13), or fragments thereof, and/or may optionally comprise up to one or up to two or up to three conservative amino acid substitutions. In certain non-limiting embodiments, the CD3ζ polypeptide can have an amino acid sequence that is a consecutive portion of SEQ ID NO: 13, which is at least about 20, or at least about 30, or at least about 40, or at least about 50, or at least about 90, or at least about 100, and up to 188 amino acids in length. Alternatively or additionally, in non-limiting various embodiments, the CD3ζ polypeptide comprises or has an amino acid sequence of amino acids 1 to 164, 1 to 50, 50 to 100, 52 to 142, 100 to 150, or 150 to 188 of SEQ ID NO: 13. In certain embodiments, the CD3ζ polypeptide comprises or has an amino acid sequence of amino acids 52 to 142 of SEQ ID NO: 13.

SEQ ID NO: 13 is provided below:

| [SEQ ID NO: 13] | |
|---|---|
| 1 | MKWKVSVLAC ILHVRFPGAE AQSFGLLDPK LCYLLDGILF IYGVIITALY LRAKFSRSAE |
| 61 | TAANLQDPNQ LYNELNLGRR EEYDVLEKKR ARDPEMGGKQ RRRNPQEGVY NALQKDKMAE |
| 121 | AYSEIGTKGE RRRGKGHDGL YQDSHFQAVQ FGNRREREGS ELTRTLGLRA RPKACRHKKP |
| 181 | LSLPAAVS |

In certain embodiments, the CD3ζ polypeptide comprises or has the amino acid sequence set forth in SEQ ID NO: 14, which is provided below:

[SEQ ID NO: 14]
RAKESRSAETAANLQDPNQLYNELNLGRREEYDVLEKKRARDPEMGGKQQ
RRRNPQEGVYNALQKDKMAEAYSEIGTKGERRRGKGHDGLYQGLSTATKD
TYDALHMQTLAPR

In accordance with the presently disclosed subject matter, a “CD3ζ nucleic acid molecule” refers to a polynucleotide encoding a CD3ζ polypeptide. In certain embodiments. the CD3ζ nucleic acid molecule encoding the CD3ζ polypeptide having the amino acid sequence set forth in SEQ ID NO: 14 comprises the nucleotide sequence set forth in SEQ ID NO: 15 as provided below.

[SEQ ID NO: 15]
AGAGCAAAATTCAGCAGGAGTGCAGAGACTGCTGCCAACCTGCAGGACCC
CAACCAGCTCTACAATGAGCTCAATCTAGGGCGAAGAGAGGAATATGACG
TCTTGGAGAAGAAGCGGGCTCGGGATCCAGAGATGGGAGGCAAACAGCAG
AGGAGGAGGAACCCCCAGGAAGGCGTATACAATGCACTGCAGAAAGACAA
GATGGCAGAAGCCTACAGTGAGATCGGCACAAAAGGCGAGAGGCGGAGAG
GCAAGGGGCACGATGGCCTTTACCAGGGTCTCAGCACTGCCACCAAGGAC
ACCTATGATGCCCTGCATATGCAGACCCTGGCCCCTCGCTAA

In certain non-limiting embodiments, an intracellular signaling domain of the CAR further comprises at least a co-stimulatory signaling region. In certain embodiments, the co-stimulatory region comprises at least one co-stimulatory molecule, which can provide optimal lymphocyte activation. As used herein, “co-stimulatory molecules” refer to cell surface molecules other than antigen receptors or their ligands that are required for an efficient response of lymphocytes to antigen. The at least one co-stimulatory signaling region can include a CD28 polypeptide, a 4-1BB polypeptide, an OX40 polypeptide, an ICOS polypeptide, a DAP-10 polypeptide, or a combination thereof. The co-stimulatory molecule can bind to a co-stimulatory ligand, which is a protein expressed on cell surface that upon binding to its receptor produces a co-stimulatory response, i.e., an intracellular response that effects the stimulation provided when an antigen binds to its CAR molecule. Co-stimulatory ligands, include, but are not limited to CD80, CD86, CD70, OX40L, and 4-1BBL. As one example, a 4-1BB ligand (i.e., 4-1BBL) may bind to 4-1BB (also known as “CD137”) for providing an intracellular signal that in combination with a CAR signal induces an effector cell function of the CAR⁺ T cell. CARs comprising an intracellular signaling domain that comprises a co-stimulatory signaling region comprising 4-1BB, ICOS or DAP-10 are disclosed in U.S. Pat. No. 7,446,190 (e.g., the nucleotide sequence encoding 4-1BB is set forth in SEQ ID NO:15, the nucleotide sequence encoding ICOS is set forth in SEQ ID NO:16, and the nucleotide sequence encoding DAP-10 is set forth in SEQ ID NO:17 in U.S. Pat. No. 7,446,190), which is herein incorporated by reference in its entirety.

In certain embodiments, the intracellular signaling domain of the CAR comprises a co-stimulatory signaling region that comprises a CD28 polypeptide. The CD28 polypeptide can have an amino acid sequence that is at least about 85%, about 90%, about 95%, about 96%, about 97%, about 98%, about 99% or 100% homologous to the sequence having a NCBI Reference No: P10747 or NP_006130 (SEQ ID NO: 2), or fragments thereof, and/or may optionally comprise up to one or up to two or up to three conservative amino acid substitutions. In non-limiting certain embodiments, the CD28 polypeptide has an amino acid sequence that is a consecutive portion of SEQ ID NO: 2 which is at least 20, or at least 30, or at least 40, or at least 50, and up to 220 amino acids in length. Alternatively or additionally, in non-limiting various embodiments, the CD28 polypeptide has an amino acid sequence of amino acids 1 to 220, 1 to 50, 50 to 100, 100 to 150, 114 to 220, 150 to 200, or 200 to 220 of SEQ ID NO: 2. In certain embodiments, the intracellular signaling domain of the CAR comprises a co-stimulatory signaling region that comprises a CD28 polypeptide having an amino acid sequence of amino acids 180 to 220 of SEQ ID NO: 2.

In certain embodiments, the CD28 polypeptide has an amino acid sequence that is at least about 85%, about 90%, about 95%, about 96%, about 97%, about 98%, about 99% or about 100% homologous to the sequence having a NCBI Reference No: NP_031668.3 (SEQ ID NO: 16), or fragments thereof, and/or may optionally comprise up to one or up to two or up to three conservative amino acid substitutions. In non-limiting certain embodiments, the CD28 polypeptide has an amino acid sequence that is a consecutive portion of SEQ ID NO: 16 which is at least about 20, or at least about 30, or at least about 40, or at least about 50, and up to 218 amino acids in length. Alternatively or additionally, in non-limiting various embodiments, the CD28 polypeptide has an amino acid sequence of amino acids 1 to 218, 1 to 50, 50 to 100, 100 to 150, 114 to 220, 150 to 200, 178 to 218, or 200 to 220 of SEQ ID NO: 16. In certain embodiments, the co-stimulatory signaling region of a presently disclosed CAR comprises a CD28 polypeptide that comprises or has the amino acids 178 to 218 of SEQ ID NO: 16.

SEQ ID NO: 16 is provided below:

| [SEQ ID NO: 16] | |
|---|---|
| 1 | MTLRLLFLAL NFFSVQVTEN KILVKQSPLL VVDSNEVSLS CRYSYNLLAK EFRASLYKGV |
| 61 | NSDVEVCVGN GNFTYQPQFR SNAEFNCDGD FDNETVTFRL WNLHVNHTDI YFCKIEFMYP |
| 121 | PPYLDNERSN GTIIHIKEKH LCHTQSSPKL FWALVVVAGV LFCYGLLVTV ALCVIWTNSR |
| 181 | RNRLLQSDYM NMTPRRPGLT RKPYQPYAPA RDFAAYRP |

In accordance with the presently disclosed subject matter, a “CD28 nucleic acid molecule” refers to a polynucleotide encoding a CD28 polypeptide. In certain embodiments, a CD28 nucleic acid molecule that encodes a CD28 polypeptide comprised in the co-stimulatory signaling region of a presently disclosed CAR (e.g., amino acids 178 to 218 of SEQ ID NO: 16) comprises or has a nucleotide sequence set forth in SEQ ID NO: 17, which is provided below.

[SEQ ID NO: 17]
AATAGTAGAAGGAACAGACTCCTTCAAAGTGACTACATGAACATGACTCC
CCGGAGGCCTGGGCTCACTCGAAAGCCTTACCAGCCCTACGCCCCTGCCA
GAGACTTTGCAGCGTACCGCCCC

In certain embodiments, the intracellular signaling domain of the CAR comprises a co-stimulatory signaling region that comprises two co-stimulatory molecules: CD28 and 4-1BB or CD28 and OX40.

4-1BB can act as a tumor necrosis factor (TNF) ligand and have stimulatory activity. The 4-1BB polypeptide can have an amino acid sequence that is at least about 85%, about 90%, about 95%, about 96%, about 97%, about 98%, about 99% or about 100% homologous to the sequence having a NCBI Reference No: P41273 or NP_001552 (SEQ ID NO: 3) or fragments thereof, and/or may optionally comprise up to one or up to two or up to three conservative amino acid substitutions.

SEQ ID NO: 3 is provided below:

| [SEQ ID NO: 3] | |
|---|---|
| 1 | MGNSCYNIVA TLLLVLNFER TRSLQDPCSN CPAGTFCDNN RNQICSPCPP NSFSSAGGQR |
| 61 | TCDICRQCKG VFRTRKECSS TSNAECDCTP GFHCLGAGCS MCEQDCKQGQ ELTKKGCKDC |
| 121 | CFGTFNDQKR GICRPWTNCS LDGKSVLVNG TKERDVVCGP SPADLSPGAS SVTPPAPARE |
| 181 | PGHSPQIISF FLALTSTALL FLLFFLTLRF SVVKRGRKKL LYIFKQPFMR PVQTTQEEDG |
| 241 | CSCRFPEEEE GGCEL |

In accordance with the presently disclosed subject matter, a “4-1BB nucleic acid molecule” refers to a polynucleotide encoding a 4-1BB polypeptide.

An OX40 polypeptide can have an amino acid sequence that is at least about 85%, about 90%, about 95%, about 96%, about 97%, about 98%, about 99% or about 100% homologous to the sequence having a NCBI Reference No: P43489 or NP_003318 (SEQ ID NO: 18), or fragments thereof, and/or may optionally comprise up to one or up to two or up to three conservative amino acid substitutions.

SEQ ID NO: 18 is provided below:

| [SEQ ID NO: 18] | |
|---|---|
| 1 | MCVGARRLGR GPCAALLLLG LGLSTVTGLH CVGDTYPSND RCCHECRPGN GMVSRCSRSQ |
| 61 | NTVCRPCGPG FYNDVVSSKP CKPCTWCNLR SGSERKQLCT ATQDTVCRCR AGTQPLDSYK |
| 121 | PGVDCAPCPP GHFSPGDNQA CKPWTNCTLA GKHTLQPASN SSDAICEDRD PPATQPQETQ |
| 181 | GPPARPITVQ PTEAWPRTSQ GPSTRPVEVP GGRAVAAILG LGLVLGLLGP LAILLALYLL |
| 241 | RRDQRLPPDA HKPPGGGSFR TPIQEEQADA HSTLAKI |

In accordance with the presently disclosed subject matter, an “OX40 nucleic acid molecule” refers to a polynucleotide encoding an OX40 polypeptide.

An ICOS polypeptide can have an amino acid sequence that is at least about 85%, about 90%, about 95%, about 96%, about 97%, about 98%, about 99% or about 100% homologous to the sequence having a NCBI Reference No: NP_036224 (SEQ ID NO: 19) or fragments thereof, and/or may optionally comprise up to one or up to two or up to three conservative amino acid substitutions.

SEQ ID NO: 19 is provided below:

| [SEQ ID NO: 19] | |
|---|---|
| 1 | MKSGLWYFFL FCLRIKVLTG EINGSANYEM FIFHNGGVQI LCKYPDIVQQ FKMQLLKGGQ |
| 61 | ILCDLIKTKG SGNTVSIKSL KFCHSQLSNN SVSFFLYNLD HSHANYYFCN LSIFDPPPFK |
| 121 | VTLIGGYLHI YESQLCCQLK FWLPIGCAAF VVVCILGCIL ICWLTKKKYS SSVHDPNGEY |
| 181 | MFMRAVNTAK KSRLTDVTL |

In accordance with the presently disclosed subject matter, an “ICOS nucleic acid molecule” refers to a polynucleotide encoding an ICOS polypeptide.

In certain embodiments, a presently disclosed CAR can further comprise an inducible promoter, for expressing nucleic acid sequences in human cells. Promoters for use in expressing CAR genes can be a constitutive promoter, such as ubiquitin C (UbiC) promoter.

In certain embodiments, a presently disclosed CAR comprises an extracellular antigen-binding domain that binds to CD19, a transmembrane domain comprising a CD28 polypeptide, and an intracellular signaling domain comprising a CD3ζ polypeptide and a co-stimulatory signaling region comprising a CD28 polypeptide. In certain embodiments, the CAR is 1928z. In certain embodiments, 1928z is a protein having at least about 85%, about 90%, about 95%, about 96%, about 97%, about 98%, about 99% or about 100% homologous to the amino acid sequence set forth in SEQ ID NO: 6, which is provided below. SEQ ID NO: 6 includes a CD8 leader sequence at amino acids 1-18, and is able to bind CD19.

MALPVTALLLPLALLLHAEVKLQQSGAELVRPGSSVKISCKASGYAFSSY
WMNWVKQRPGQGLEWIGQIYPGDGDTNYNGKFKGQATLTADKSSSTAYMQ
LSGLTSEDSAVYFCARKTISSVVDFYFDYWGQGTTVTVSSGGGGSGGGGS
GGGGSDIELTQSPKFMSTSVGDRVSVTCKASQNVGTNVAWYQQKPGQSPK
PLIYSATYRNSGVPDRFTGSGSGTDFTLTITNVQSKDLADYFCQQYNRYP
YTSGGGTKLEIKRAAAIEVMYPPPYLDNEKSNGTIIHVKGKHLCPSPLFP
GPSKPFWVLVVVGGVLACYSLLVTVAFIIFWVRSKRSRLLHSDYMNMTPR
RPGPTRKHYQPYAPPRDFAAYRSRVKFSRSAEPPAYQQGQNQLYNELNLG
RREEYDVLDKRRGRDPEMGGKPRRKNPQEGLYNELQKDKMAEAYSEIGMK
GERRRGKGHDGLYQGLSTATKDTYDALHMQALPPRX

An exemplary nucleic acid sequence encoding a 1928z polypeptide, including a CD8 leader sequence, is set forth in SEQ ID NO:7, which is provided below.

(SEQ ID NO: 7)
ccatggctctcccagtgactgccctactgcttcccctagcgcttctcctg
catgcagaggtgaagctgcagcagtctggggctgagctggtgaggcctgg
gtcctcagtgasgatttcctgcaaggcttctggctatgcattcagtagct
actggatgaactgggtgaagcagaggcctggacagggtcttgagtggatt
ggacagatttatcctggagatggtgatactaactacaatggaaagttcaa
gggtcaagccacactgactgcagacaaatcctccagcacagcctacatgc
agctcagcggcctaacatctgaggactctgcggtctatttctgtgcaaga
aagaccattagttcggtagtagatttctactttgactactggggccaagg
gaccacggtcaccgtctcctcaggtggaggtggatcaggtggaggtggat
ctggtggaggtggatctgacattgagctcacccagtctccaaaattcatg
tccacatcagtaggagacagggtcagcgtcacctgcaaggccagtcagaa
tgtgggtactaatgtagcctggtatcaacagaaaccaggacaatctccta
aaccactgatttactcggcaacctaccggaacagtggagtccctgatcgc
ttcacaggcagtggatctgggacagatttcactctcaccatcactaacgt
gcagtctaaagacttggcagactatttctgtcaacaatataacaggtatc
cgtacacgtccggaggggggaccaagctggagatcaaacgggcggccgca
attgaagttatgtatcctcctccttacctagacaatgagaagagcaatgg
aaccattatccatgtgaaagggaaacacctttgtccaagtcccctatttc
ccggaccttctaagcccttttgggtgctggtggtggttggtggagtcctg
gcttgctatagcttgctagtaacagtggcctttattattttctgggtgag
gagtaagaggagcaggctcctgcacagtgactacatgaacatgactcccc
gccgccccgggcccacccgcaagcattaccagccctatgccccaccacgc
gacttcgcagcctatcgctccagagtgaagttcagcaggagcgcagagcc
ccccgcgtaccagcagggccagaaccagctctataacgagctcaatctag
gacgaagagaggagtacgatgttttggacaagagacgtggccgggaccct
gagatggggggaaagccgagaaggaagaaccctcaggaaggcctgtacaa
tgaactgcagaaagataagatggcggaggcctacagtgagattgggatga
aaggcgagcgccggaggggcaaggggcacgatggcctttaccagggtctc
agtacagccaccaaggacacctacgacgcccttcacatgcaggccctgcc
ccctcgcg

In certain embodiments, a presently disclosed CAR comprises an extracellular antigen-binding domain that binds to MUC16, a transmembrane domain comprising a CD28 polypeptide, and an intracellular signaling domain comprising a CD3ζ polypeptide and a co-stimulatory signaling region comprising a CD28 polypeptide. In certain embodiments, the CAR is 4H1128z. In certain embodiments, 4H1128z is a protein having at least about 85%, about 90%, about 95%, about 96%, about 97%, about 98%, about 99% or about 100% homologous to the amino acid sequence set forth in SEQ ID NO: 20, which is provided below. SEQ ID NO: 20 includes a CD8 leader sequence at amino acids 1-18, and is able to bind to MUC-16 ectodomain.

(SEQ ID NO: 20)
MALPVTALLLPLALLLHAEVKLQESGGGFVKPGGSLKVSCAASGFTFSSY
AMSWVRLSPEMRLEWVATISSAGGYIFYSDSVQGRFTISRDNAKNTLHLQ
MGSLRSGDTAMYYCARQGFGNYGDYYAMDYWGQGTTVTVSSGGGGSGGGG
SGGGGSDIELTQSPSSLAVSAGEKVTMSCKSSQSLLNSRTRKNQLAWYQQ
KPGQSPELLIYWASTRQSGVPDRFTGSGSGTDFTLTISSVQAEDLAVYYC
QQSYNLLTFGPGTKLEIKRAAAIEVMYPPPYLDNEKSNGTIIHVKGKHLC
PSPLFPGPSKPFWVLVVVGGVLACYSLLVTVAFIIFWVRSKRSRLLHSDY
MNMTPRRPGPTRKHYQPYAPPRDFAAYRSRVKFSRSAEPPAYQQGQNQLY
NELNLGRREEYDVLDKRRGRDPEMGGKPRRKNPQEGLYNELQKDKMAEAY
SEIGMKGERRRGKGHDGLYQGLSTATKDTYDALHMQALPPR

An exemplary nucleic acid sequence encoding a 4H1128z polypeptide, including a Kappa leader sequence, is set forth in SEQ ID NO: 21, which is provided below.

(SEQ ID NO: 21)
ccatggctctcccagtgactgccctactgcttcccctagcgcttctcctg
catgcagaggtgaagctgcaggagtcagggggaggcttcgtgaagcctgg
agggtccctcaaagtctcctgtgcagcctctggattcactttcagtagct
atgccatgtcctgggttcgcctgagtccggagatgaggctggagtgggtc
gcaaccattagcagtgctggtggttacatcttctattctgacagcgtgca
gggacgattcaccatttccagagacaatgccaagaacaccctgcacctgc
aaatgggcagtctgaggtctggggacacggccatgtattactgtgcaagg
cagggatttggtaactacggtgattactatgctatggactactggggcca
agggaccacggtcaccgtctcctcaggtggaggtggatcaggtggaggtg
gatctggtggaggtggatctgacattgagctcacccagtctccatcctcc
ctggctgtgtcagcaggagagaaggtcactatgagctgcaaatccagtca
gagtctgctcaacagtagaacccgaaagaaccagttggcttggtaccagc
aaaaaccaggacagtctcctgaactgctgatctactgggcatccactagg
caatctggagtccctgatcgcttcacaggcagtggatctgggacagattt
cactctcaccatcagcagtgtgcaggctgaagacetggcagtttattact
gccagcaatcttataatctactcacgttcggtcctgggaccaagctggag
atcaaacgggcggccgcaattgaagttatgtatcctcctccttacctaga
caatgagaagagcaatggaaccattatccatgtgaaagggaaacaccttt
gtccaagtcccctatttcccggaccttctaagcccttttgggtgctggtg
gtggttggtggagtcctggcttgctatagcttgctagtaacagtggccct
tattattttctgggtgaggagtaagaggagcaggctcctgcacagtgact
acatgaacatgactccccgccgccccgggcccacccgcaagcattaccag
ccctatgccccaccacgcgacttcgcagcctatcgctccagagtgaagtt
cagcaggagcgcagagccccccgcgtaccagcagggccagaaccagctct
ataacgagctcaatctaggacgaagagaggagtacgatgttttggacaag
agacgtggccgggaccctgagatggggggaaagccgagaaggaagaaccc
tcaggaaggcctgtacaatgaactgcagaaagataagatggcggaggcct
acagtgagattgggatgaaaggcgagcgccggaggggcaaggggcacgat
ggcctttaccagggtctcagtacagccaccaaggacacctacgacgccct
tcacatgcaggccctgccccctcgc

In certain embodiments, a presently disclosed CAR comprises an extracellular antigen-binding domain that binds to CD19, a transmembrane domain comprising a CD8 polypeptide, and an intracellular signaling domain comprising a CD3ζ polypeptide and a co-stimulatory signaling region comprising a 4-1BB polypeptide. In certain embodiments, the CAR is 19BBz.

An exemplary nucleic acid sequence encoding an 19BBz polypeptide is set forth in SEQ ID NO: 30, which is provided below.

(SEQ ID NO: 30)
ATGGCTCTCCCAGTGACTGCCCTACTGCTTCCCCTAGCGCTTCTCCTGCA
TGCAGAGGTGAAGCTGCAGCAGTCTGGGGCTGAGCTGGTGAGGCCTGGGT
CCTCAGTGAAGATTTCCTGCAAGGCTTCTGGCTATGCATTCAGTAGCTAC
TGGATGAACTGGGTGAAGCAGAGGCCTGGACAGGGTCTTGAGTGGATTGG
ACAGATTTATCCTGGAGATGGTGATACTAACTACAATGGAAAGTTCAAGG
GTCAAGCCACACTGACTGCAGACAAATCCTCCAGCACAGCCTACATGCAG
CTCAGCGGCCTAACATCTGAGGACTCTGCGGTCTATTTCTGTGCAAGAAA
GACCATTAGTTCGGTAGTAGATTTCTACTTTGACTACTGGGGCCAAGGGA
CCACGGTCACCGTCTCCTCAGGTGGAGGTGGATCAGGTGGAGGTGGATCT
GGTGGAGGTGGATCTGACATTGAGCTCACCCAGTCTCCAAAATTCATGTC
CACATCAGTAGGAGACAGGGTCAGCGTCACCTGCAAGGCCAGTCAGAATG
TGGGTACTAATGTAGCCTGGTATCAACAGAAACCAGGACAATCTCCTAAA
CCACTGATTTACTCGGCAACCTACCGGAACAGTGGAGTCCCTGATCGCTT
CACAGGCAGTGGATCTGGGACAGATTTCACTCTCACCATCACTAACGTGC
AGTCTAAAGACTTGGCAGACTATTTCTGTCAACAATATAACAGGTATCCG
TACACGTCCGGAGGGGGGACCAAGCTGGAGATCAAACGGGCGGCCGCACC
CACCACGACGCCAGCGCCGCGACCACCAACCCCGGCGCCCACGATCGCGT
CGCAGCCCCTGTCCCTGCGCCCAGAGGCGTGCCGGCCAGCGGCGGGGGGC
GCAGTGCACACGAGGGGGCTGGACTTCGCCTGTGATATCTACATCTGGGC
GCCCCTGGCCGGGACTTGTGGGGTCCTTCTCCTGTCACTGGTTATCACCC
TTTACTGCAACAAACGGGGCAGAAAGAAGCTCCTGTATATATTCAAACAA
CCATTTATGAGACCAGTACAAACTACTCAAGAGGAAGATGGCTGTAGCTG
CCGATTTCCAGAAGAAGAAGAAGGAGGATGTGAACTGAGAGTGAAGTTCA
GCAGGAGCGCAGAGCCCCCCGCGTACCAGCAGGGCCAGAACCAGCTCTAT
AACGAGCTCAATCTAGGACGAAGAGAGGAGTACGATGTTTTGGACAAGAG
ACGTGGCCGGGACCCTGAGATGGGGGGAAAGCCGAGAAGGAAGAACCCTC
AGGAAGGCCTGTACAATGAACTGCAGAAAGATAAGATGGCGGAGGCCTAC
AGTGAGATTGGGATGAAAGGCGAGCGCCGGAGGGGCAAGGGGCACGATGG
CCTTTACCAGGGTCTCAGTACAGCCACCAAGGACACCTACGACGCCCTTC
ACATGCAGGCCCTGCCCCCTCGCTAA

The presently disclosed subject matter also provides a nucleic acid comprising a first nucleic acid sequence encoding an antigen recognizing receptor and a second nucleic acid sequence encoding an exogenous IL-18 polypeptide. In certain embodiments, a nucleic acid comprising a first nucleic acid sequence encoding an 19BBz CAR and a second nucleic acid sequence encoding an exogenous IL-18 polypeptide comprises the nucleic acid sequence set forth in SEQ ID NO: 31, which is provided below, wherein nucleic acids 1 to 1473 of SEQ ID NO: 31 encode 19BBz CAR, and nucleic acids 1540 to 2198 encode the exogenous IL-18 polypeptide.

(SEQ ID NO: 31)
ATGGCTCTCCCAGTGACTGCCCTACTGCTTCCCCTAGCGCTTCTCCTGCA
TGCAGAGGTGAAGCTGCAGCAGTCTGGGGCTGAGCTGGTGAGGCCTGGGT
CCTCAGTGAAGATTTCCTGCAAGGCTTCTGGCTATGCATTCAGTAGCTAC
TGGATGAACTGGGTGAAGCAGAGGCCTGGACAGGGTCTTGAGTGGATTGG
ACAGATTTATCCTGGAGATGGTGATACTAACTACAATGGAAAGTTCAAGG
GTCAAGCCACACTGACTGCAGACAAATCCTCCAGCACAGCCTACATGCAG
CTCAGCGGCCTAACATCTGAGGACTCTGCGGTCTATTTCTGTGCAAGAAA
GACCATTAGTTCGGTAGTAGATTTCTACTTTGACTACTGGGGCCAAGGGA
CCACGGTCACCGTCTCCTCAGGTGGAGGTGGATCAGGTGGAGGTGGATCT
GGTGGAGGTGGATCTGACATTGAGCTCACCCAGTCTCCAAAATTCATGTC
CACATCAGTAGGAGACAGGGTCAGCGTCACCTGCAAGGCCAGTCAGAATG
TGGGTACTAATGTAGCCTGGTATCAACAGAAACCAGGACAATCTCCTAAA
CCACTGATTTACTCGGCAACCTACCGGAACAGTGGAGTCCCTGATCGCTT
CACAGGCAGTGGATCTGGGACAGATTTCACTCTCACCATCACTAACGTGC
AGTCTAAAGACTTGGCAGACTATTTCTGTCAACAATATAACAGGTATCCG
TACACGTCCGGAGGGGGGACCAAGCTGGAGATCAAACGGGCGGCCGCACC
CACCACGACGCCAGCGCCGCGACCACCAACCCCGGCGCCCACGATCGCGT
CGCAGCCCCTGTCCCTGCGCCCAGAGGCGTGCCGGCCAGCGGCGGGGGGC
GCAGTGCACACGAGGGGGCTGGACTTCGCCTGTGATATCTACATCTGGGC
GCCCCTGGCCGGGACTTGTGGGGTCCTTCTCCTGTCACTGGTTATCACCC
TTTACTGCAACAAACGGGGCAGAAAGAAGCTCCTGTATATATTCAAACAA
CCATTTATGAGACCAGTACAAACTACTCAAGAGGAAGATGGCTGTAGCTG
CCGATTTCCAGAAGAAGAAGAAGGAGGATGTGAACTGagagtgaagttca
gcaggagcgcagacgcccccgcgtaccagcagggccagaaccagctctat
aacgagctcaatctaggacgaagagaggagtacgatgttttggacaagag
acgtggccgggaccctgagatggggggaaagccgagaaggaagaaccctc
aggaaggcctgtacaatgaactgcagaaagataagatggcggaggcctac
agtgagattgggatgaaaggcgagcgccggaggggcaaggggcacgatgg
cctttaccagggtctcagtacagccaccaaggacacctacgacgcccttc
acatgcaggccctgccccctcgcggatctggagcaacaaacttctcacta
ctcaaacaagcaggtgacgtggaggagaatcccggacCCATGGGTTACAG
GATGCAACTCCTGTCTTGCATTGCACTAAGTCTTGCACTTGTCACAAACA
GTGGCTACTTTGGCAAGCTTGAATCTAAATTATCAGTCATAAGAAATTTG
AATGACCAAGTTCTCTTCATTGACCAAGGAAATCGGCCTCTATTTGAAGA
TATGACTGATTCTGACTGTAGAGATAATGCACCCCGGACCATATTTATTA
TAAGTATGTATAAAGATAGCCAGCCTAGAGGTATGGCTGTAACTATCTCT
GTGAAGTGTGAGAAAATTTCAACTCTCTCCTGTGAGAACAAAATTATTTC
CTTTAAGGAAATGAATCCTCCTGATAACATCAAGGATACAAAAAGTGACA
TCATATTCTTTCAGAGAAGTGTCCCAGGACATGATAATAAGATGCAATTT
GAATCTTCATCATACGAAGGATACTTTCTAGCTTGTGAAAAAGAGAGAGA
CCTTTTTAAACTCATTTTGAAAAAAGAGGATGAATTGGGGGATAGATCTA
TAATGTTCACTGTTCAAAACGAAGACtagGTCGAGGATCCGGATTAGTCC
AATTTGTTAAAGACAGGATATCAGTGGTCCAGGCTCTAGTTTTGACTCAA
CAATATCACCAGCTGAAGCCTATAGAGTACGAGCCATAGATAAAATAA

### 3. Immunoresponsive Cells

The presently disclosed subject matter provides immunoresponsive cells comprising (a) an antigen recognizing receptor (e.g., CAR or TCR), and (b) a secretable IL-18 polypeptide. In certain embodiments, the secretable IL-18 polypeptide is an exogenous IL-18 polypeptide. In certain embodiments, the antigen recognizing receptor is capable of activating the immunoresponsive cell. In certain embodiments, the secretable IL-18 polypeptide (e.g., exogenous IL-18 polypeptide) is capable of promoting an anti-tumor effect of the immunoresponsive cell. The immunoresponsive cells can be transduced with an antigen recognizing receptor and an exogenous IL-18 polypeptide such that the cells express the antigen recognizing receptor and the exogenous IL-18 polypeptide. The presently disclosed subject matter also provides methods of using such cells for treating and/or preventing treating and/or preventing a disease that requires an enhanced immune response, e.g., a liquid or solid tumor.

The immunoresponsive cells of the presently disclosed subject matter can be cells of the lymphoid lineage. The lymphoid lineage, comprising B, T and natural killer (NK) cells, provides for the production of antibodies, regulation of the cellular immune system, detection of foreign agents in the blood, detection of cells foreign to the host, and the like. Non-limiting examples of immunoresponsive cells of the lymphoid lineage include T cells, Natural Killer (NK) cells, embryonic stem cells, and pluripotent stem cells (e.g., those from which lymphoid cells may be differentiated). T cells can be lymphocytes that mature in the thymus and are chiefly responsible for cell-mediated immunity. T cells are involved in the adaptive immune system. The T cells of the presently disclosed subject matter can be any type of T cells, including, but not limited to, helper T cells, cytotoxic T cells, memory T cells (including central memory T cells, stem-cell-like memory T cells (or stem-like memory T cells), and two types of effector memory T cells: e.g., T_{EM} cells and T_{EMRA} cells, Regulatory T cells (also known as suppressor T cells), Natural killer T cells, Mucosal associated invariant T cells, and γδ T cells. Cytotoxic T cells (CTL or killer T cells) are a subset of T lymphocytes capable of inducing the death of infected somatic or tumor cells. A patient's own T cells may be genetically modified to target specific antigens through the introduction of an antigen recognizing receptor, e.g., a CAR or a TCR. In certain embodiments, the immunoresponsive cell is a T cell. The T cell can be a CD4⁺ T cell or a CD8⁺ T cell. In certain embodiments, the T cell is a CD4⁺ T cell. In certain embodiments, the T cell is a CD8⁺ T cell.

Natural killer (NK) cells can be lymphocytes that are part of cell-mediated immunity and act during the innate immune response. NK cells do not require prior activation in order to perform their cytotoxic effect on target cells.

Types of human lymphocytes of the presently disclosed subject matter include, without limitation, peripheral donor lymphocytes, e.g., those disclosed in Sadelain, M., et al. 2003 Nat Rev Cancer 3:35-45 (disclosing peripheral donor lymphocytes genetically modified to express CARs), in Morgan, R.A., et al. 2006 Science 314:126-129 (disclosing peripheral donor lymphocytes genetically modified to express a full-length tumor antigen-recognizing T cell receptor complex comprising the α and β heterodimer), in Panelli, M. C., et al. 2000 J Immunol 164:495-504; Panelli, M. C., et al. 2000 J Immunol 164:4382-4392 (disclosing lymphocyte cultures derived from tumor infiltrating lymphocytes (TILs) in tumor biopsies), and in Dupont, J., et al. 2005 Cancer Res 65:5417-5427; Papanicolaou, G. A., et al. 2003 Blood 102:2498-2505 (disclosing selectively in vitro-expanded antigen-specific peripheral blood leukocytes employing artificial antigen-presenting cells (AAPCs) or pulsed dendritic cells). The immunoresponsive cells (e.g., T cells) can be autologous, non-autologous (e.g., allogeneic), or derived in vitro from engineered progenitor or stem cells.

The presently disclosed immunoresponsive cells are capable of modulating the tumor microenvironment. Tumors have a microenvironment that is hostile to the host immune response involving a series of mechanisms by malignant cells to protect themselves from immune recognition and elimination. This “hostile tumor microenvironment” comprises a variety of immune suppressive factors including infiltrating regulatory CD4⁺ T cells (Tregs), myeloid derived suppressor cells (MDSCs), tumor associated macrophages (TAMs), immune suppressive cytokines including IL-10 and TGF-β, and expression of ligands targeted to immune suppressive receptors expressed by activated T cells (CTLA-4 and PD-1). These mechanisms of immune suppression play a role in the maintenance of tolerance and suppressing inappropriate immune responses, however within the tumor microenvironment these mechanisms prevent an effective anti-tumor immune response. Collectively these immune suppressive factors can induce either marked anergy or apoptosis of adoptively transferred CAR modified T cells upon encounter with targeted tumor cells.

In certain embodiments, the presently disclosed immunoresponsive cells have increased secretion of anti-tumor cytokines, including, but not limited to, IL-18, IL-2, IFN-γ, and TNF-α. In certain embodiments, the immunoresponsive cells have decreased secretion of cytokines associated with cytokine release syndrome (CRS), e.g., IL-6.

### Interleukin-18

Interleukin 18 (also known as IGIF, IL-1g and IL1F4; GenBank ID: 3606 (human), 16173 (mouse), 29197 (rat), 403796 (dog), 100034216 (horse), 281249(cattle)) is a gene encoding a pro-inflammatory cytokine that increases immune activity of certain immunoresponsive cells. The protein product of Interleukin 18 includes, but is not limited to, NCBI Reference Sequences NP_001553.1 and NP_001230140.1. IL-18 is produced by macrophages, T cells and other cells. IL-18 functions by binding to the interleukin-18 receptor, and together with other cytokines, such as IL-12, it can induce cell-mediated immunity, which following infection with microbial products. After stimulation with IL-18, natural killer (NK) cells and certain T cells release other cytokines, such as interferon-γ (IFN-γ), IL-2 and TNF-α, which can further activate other types of immunoresponsive cells.

In certain embodiments, the term “IL-18” or “IL-18 cytokine” refers to the bioactive form of IL-18 after secretion from a cell (that is to say, where the signal peptide has been cleaved off). A non-limiting example of human IL-18 has the following amino acid sequence set forth in SEQ ID NO: 4, which is provided below.

(SEQ ID NO: 4)
YFGKLESKLSVIRNLNDQVLFIDQGNRPLFEDMTDSDCRDNAPRTIFIIS
MYKDSQPRGMAVTISVKCEKISTLSCENKIISFKEMNPPDNIKDTKSDII
FFQRSVPGHDNKMQFESSSYEGYFLACEKERDLEKLILKKEDELGDRSIM
FTVQNED

In certain embodiments, a secretable IL-18 polypeptide refers to a polypeptide or a protein, the cytokine portion of which has at least about 85%, about 90%, about 95%, about 96%, about 97%, about 98%, about 99%, or about 100% homologous to the cytokine portion of the protein product of IL-18 (GenBank ID: 3606 (human), 16173 (mouse), 29197 (rat), 403796 (dog), 100034216 (horse), 281249(cattle)), or a fragment thereof that has immunostimulatory activity. In certain non-limiting embodiments, the secretable IL-18 polypeptide comprises a cytokine portion and a signal peptide, optionally joined by a linker peptide. Non-limiting examples of secretable IL-18 polypeptides include NCBI Reference Sequences NP_001553.1 and NP_001230140.1. An exemplary secretable IL-18 polypeptide is provided below [SEQ ID NO: 5].

| | |
|---|---|
| 1 | maaepvednc infvamkfid ntlyfiaedd enlesdyfgk lesklsvirn lndqvlfidq |
| 61 | gnrplfedmt dsdcrdnapr tifiismykd sqprgmavti svkcekistl scenkiisfk |
| 121 | emnppdnikd tksdiiffqr svpghdnkmq fesssyegyf lacekerdlf klilkkedel |
| 181 | gdrsimftvq ned |

In certain non-limiting embodiments, the secretable IL-18 polypeptide comprises a signal sequence, for example, but not limited to the IL-2 signal sequence, the kappa leader sequence, the CD8 leader sequence or a peptide with essentially equivalent activity.

### IL-2

Interleukin 2 (IL-2, GenBank ID: 3558, also known as TCGA and lymphokine) is a gene encoding a secreted cytokine that regulates the activities of immunoresponsive cells. IL-2 mediates its effects by binding to IL-2 receptors, a heterotrimeric protein complex expressed by lymphocytes. IL-2 promotes the proliferation of T and B lymphocytes, and also promotes the differentiation of a T cell precursor into an effector T cell or a memory T cell, when the T cell precursor is stimulated by an antigen.

### IFN-γ

Interferon gamma (IFN-γ, GenBank ID: 3458, also known as IFNG, IFNg, IFG and IFI) is a gene encoding a soluble cytokine of the type II interferon class. IFN-γ is secreted by cells of both the innate and adaptive immune systems, such as NK cells and T cells. The protein forms a homo-dimer which binds to the interferon gamma receptor complex. The binding activates a downstream signal that triggers immune response to viral and microbial infections. IFN-γ also potentiates the effects of the type I interferons and stimulates leukocytes and macrophages, which results in increased inflammation.

### TNF-α

Tumor necrosis factor-α, GenBank ID: 7124 (also known as DIF, TNFA, TNFSF2, TNLG1F, TNF-alpha, TNFα and tumor necrosis factor alpha) is a gene encoding a multifunctional pro-inflammatory cytokine. It can be produced by macrophages, T lymphocytes, NK cells, neutrophils, mast cells, eosinophils, and neurons. The protein forms a homo-trimer which binds to two receptors, TNFR1 and TNFR2. Upon binding of TNF-α, TNF receptors also form trimers, and activate downstream signals such as NF-κB and MAPK pathways.

### IL-6

Interleukin 6 (IL-6, GenBank ID: 3569, also known as CDF, HGF, HSF, BSF2, IL-6, BSF-2, IFNB2, and IFN-beta-2) is a gene encoding a cytokine that functions in inflammation and B cells maturation. IL-6 is also capable of inducing fever in people with infections or autoimmune diseases. Increased level of circulating IL-6 is associated with Cytokine Release Syndrome (CRS, also known as cytokine-associated toxicity), a life-threatening toxicity that has been observed following administration of antibodies and adoptive T-cell therapies. Studies have demonstrated that immunosuppression using anti-IL-6 receptor antibody can reverse the syndrome.

In certain embodiments, the presently disclosed immunoresponsive cells exhibit enhanced expansion and persistence. In certain embodiments, the immunoresponsive cells comprising an antigen recognizing receptor and a secretable IL-18 polypeptide exhibit at least about 5%, about 10%, about 20%, about 30%, about 40%, about 50%, about 60%, about 70%, about 80%, about 90%, about 100%, about 150%, about 200%, about 250%, about 300%, about 400%, about 500%, about 600%, about 700%, about 800%, about 900%, about 1000%, or more increase of cell expansion compared to the immunoresponsive cells comprising an antigen recognizing receptor alone. In certain embodiments, the immunoresponsive cells comprising an antigen recognizing receptor and a secretable IL-18 polypeptide (e.g., exogenous IL-18 polypeptide) exhibit at least about 5%, about 10%, about 20%, about 30%, about 40%, about 50%, about 60%, about 70%, about 80%, about 90%, about 100%, about 150%, about 200%, about 250%, about 300%, about 400%, about 500%, about 600%, about 700%, about 800%, about 900%, about 1000%, or more increase of cell persistence compared to the immunoresponsive cells expressing comprising an antigen recognizing receptor alone.

In certain embodiments, the presently disclosed immunoresponsive cells induce prolonged B-cell aplasia. In certain embodiments, the immunoresponsive cells comprising an antigen recognizing receptor and a secretable IL-18 polypeptide (e.g., exogenous IL-18 polypeptide) decrease B-cell population by at least about 5%, about 10%, about 20%, about 30%, about 40%, about 50%, about 60%, about 70%, about 80%, about 90%, or about 100%, compared to the immunoresponsive cells expressing comprising an antigen recognizing receptor alone.

In certain embodiments, the presently disclosed immunoresponsive cells activate endogenous immune cells. In certain embodiments, the endogenous immune cells are selected from the group consisting of NK cells, NK-T cells, dendritic cells and endogenous CD8 T cells. In certain embodiments, the endogenous immune cells are endogenous CD8 T cells with a central memory phenotype (CD44⁺;Ly6C⁺) (p=0.01), macrophages with an M1 phenotype (MHC-II⁺) (p<0.0001) or dendritic cells with a mature and activated phenotype (CD86⁺;MHC-II⁺). In certain embodiments, the immunoresponsive cells expressing disclosed herein increase the endogenous immune cells population by at least about 5%, about 10%, about 20%, about 30%, about 40%, about 50%, about 60%, about 70%, about 80%, about 90%, about 100%, about 150%, about 200%, about 250%, about 300%, about 400%, about 500%, about 600%, about 700%, about 800%, about 900%, about 1000%, or more compared to the immunoresponsive cells expressing comprising an antigen recognizing receptor alone. In certain embodiments, the presently disclosed immunoresponsive cells recruit the endogenous immune cells to the tumor site.

The unpurified source of CTLs may be any known in the art, such as the bone marrow, fetal, neonate or adult or other hematopoietic cell source, e.g., fetal liver, peripheral blood or umbilical cord blood. Various techniques can be employed to separate the cells. For instance, negative selection methods can remove non-CTLs initially. mAbs are particularly useful for identifying markers associated with particular cell lineages and/or stages of differentiation for both positive and negative selections.

A large proportion of terminally differentiated cells can be initially removed by a relatively crude separation. For example, magnetic bead separations can be used initially to remove large numbers of irrelevant cells. Preferably, at least about 80%, usually at least 70% of the total hematopoietic cells will be removed prior to cell isolation.

Procedures for separation include, but are not limited to, density gradient centrifugation; resetting; coupling to particles that modify cell density; magnetic separation with antibody-coated magnetic beads; affinity chromatography; cytotoxic agents joined to or used in conjunction with a mAb, including, but not limited to, complement and cytotoxins; and panning with antibody attached to a solid matrix, e.g. plate, chip, elutriation or any other convenient technique.

Techniques for separation and analysis include, but are not limited to, flow cytometry, which can have varying degrees of sophistication, e.g., a plurality of color channels, low angle and obtuse light scattering detecting channels, impedance channels.

The cells can be selected against dead cells, by employing dyes associated with dead cells such as propidium iodide (PI). Preferably, the cells are collected in a medium comprising 2% fetal calf serum (FCS) or 0.2% bovine serum albumin (BSA) or any other suitable, preferably sterile, isotonic medium.

### 4. Vectors

Genetic modification of immunoresponsive cells (e.g., T cells, CTL cells, NK cells) can be accomplished by transducing a substantially homogeneous cell composition with a recombinant DNA construct. Preferably, a retroviral vector (either gamma-retroviral or lentiviral) is employed for the introduction of the DNA construct into the cell. For example, a polynucleotide encoding a receptor that binds an antigen (e.g., a tumor antigen, or a variant, or a fragment thereof), can be cloned into a retroviral vector and expression can be driven from its endogenous promoter, from the retroviral long terminal repeat, or from a promoter specific for a target cell type of interest. Non-viral vectors may be used as well.

For initial genetic modification of the cells to provide antigen recognizing receptors (e.g., CARs or TCRs), a retroviral vector is generally employed for transduction, however any other suitable viral vector or non-viral delivery system can be used. For genetic modification of the cells to provide cells comprising an antigen recognizing receptor and a secretable IL-18 polypeptide (e.g., exogenous IL-18 polypeptide), retroviral gene transfer (transduction) likewise proves effective. The antigen recognizing receptor (e.g., CAR or TCR) and IL-18 polypeptide can be constructed in a single, multicistronic expression cassette, in multiple expression cassettes of a single vector, or in multiple vectors. Examples of elements which create polycistronic expression cassette include, but is not limited to, various viral and non-viral Internal Ribosome Entry Sites (IRES, e.g., FGF-1 IRES, FGF-2 IRES, VEGF IRES, IGF-II IRES, NF-κB IRES, RUNX1 IRES, p53 IRES, hepatitis A IRES, hepatitis C IRES, pestivirus IRES, aphthovirus IRES, picornavirus IRES, poliovirus IRES and encephalomyocarditis virus IRES) and cleavable linkers (e.g., 2A peptides, e.g., P2A, T2A, E2A and F2A peptides). Combinations of retroviral vector and an appropriate packaging line are also suitable, where the capsid proteins will be functional for infecting human cells. Various amphotropic virus-producing cell lines are known, including, but not limited to, PA12 (Miller, et al. (1985) Mol. Cell. Biol. 5:431-437); PA317 (Miller, et al. (1986) Mol. Cell. Biol. 6:2895-2902); and CRIP (Danos, et al. (1988) Proc. Natl. Acad. Sci. USA 85:6460-6464). Non-amphotropic particles are suitable too, e.g., particles pseudotyped with VSVG, RD114 or GALV envelope and any other known in the art.

Possible methods of transduction also include direct co-culture of the cells with producer cells, e.g., by the method of Bregni, et al. (1992) Blood 80:1418-1422, or culturing with viral supernatant alone or concentrated vector stocks with or without appropriate growth factors and polycations, e.g., by the method of Xu, et al. (1994) Exp. Hemat. 22:223-230; and Hughes, et al. (1992) J. Clin. Invest. 89:1817.

Other transducing viral vectors can be used to express a antigen receptor, a secretable IL-18 polypeptide, and/or other components of the invention in an immunoresponsive cell. Preferably, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Other viral vectors that can be used include, for example, adenoviral, lentiviral, and adena-associated viral vectors, vaccinia virus, a bovine papilloma virus, or a herpes virus, such as Epstein-Barr Virus (also see, for example, the vectors of Miller, Human Gene Therapy 15-14, 1990; Friedman, Science 244: 1275-1281, 1989; Eglitis et al., BioTechniques 6:608-614, 1988; Tolstoshey et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson. Science 226:401-409, 1984; Moen, Blood Cells 17:407-416, 1991; Miller et al., Biotechnology 7:980-990, 1989; LeGal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346).

Non-viral approaches can also be employed for the expression of a protein in cell. For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases or targeted nucleases (e.g. Zinc finger nucleases, meganucleases, or TALE nucleases). Transient expression may be obtained by RNA electroporation.

cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g. the elongation factor 1a enhancer promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

The resulting cells can be grown under conditions similar to those for unmodified cells, whereby the modified cells can be expanded and used for a variety of purposes.

### 5. Enhancing Endogenous IL-18 Gene Expression

Any targeted genome editing methods can be used to modify the promoter/enhancer region of the IL-18 gene locus, and thereby enhance the endogenous expression of IL-18 in an immunoresponsive cell. In certain embodiments, a constitutive promoter can be placed to the IL-18 gene locus to drive IL-18 gene expression. Eligible constitutive promoters include, but are not limited to, a CMV promoter, a EF1a promoter, a SV40 promoter, a PGK1 promoter, a Ubc promoter, a beta-actin promoter, and a CAG promoter. Alternatively, a conditional or inducable promoter can be placed to the IL-18 gene locus to drive IL-18 gene expression. Examples of conditional promoters include, but is not limited to, a tetracycline response element (TRE) promoter and an estrogen response element (ERE) promoter. In addition, enhancer elements can be placed in regions other than the promoter region.

### 6. Gene Editing Methods

Any targeted genome editing methods can be used to modify the promoter enhancer region of the IL-18 gene locus.

In certain embodiments, the CRISPR system is used to modify the promoter/enhancer region of the IL-18 gene locus. Clustered regularly-interspaced short palindromic repeats (CRISPR) system is a genome editing tool discovered in prokaryotic cells. When utilized for genome editing, the system includes Cas9 (a protein able to modify DNA utilizing crRNA as its guide), CRISPR RNA (crRNA, contains the RNA used by Cas9 to guide it to the correct section of host DNA along with a region that binds to tracrRNA (generally in a hairpin loop form) forming an active complex with Cas9), trans-activating crRNA (tracrRNA, binds to erRNA and forms an active complex with Cas9), and an optional section of DNA repair template (DNA that guides the cellular repair process allowing insertion of a specific DNA sequence). CRISPR/Cas9 often employs a plasmid to transfect the target cells. The crRNA needs to be designed for each application as this is the sequence that Cas9 uses to identify and directly bind to the target DNA in a cell. The repair template carrying CAR expression cassette need also be designed for each application, as it must overlap with the sequences on either side of the cut and code for the insertion sequence. Multiple crRNA's and the tracrRNA can be packaged together to form a single-guide RNA (sgRNA). This sgRNA can be joined together with the Cas9 gene and made into a plasmid in order to be transfected into cells.

In certain embodiments, zinc-finger nucleases are used to modify the promoter/enhancer region of the IL-18 gene locus. A zinc-finger nuclease (ZFN) is an artificial restriction enzyme, which is generated by combining a zinc finger DNA-binding domain with a DNA-cleavage domain. A zinc finger domain can be engineered to target specific DNA sequences which allows a zinc-finger nuclease to target desired sequences within genomes. The DNA-binding domains of individual ZFNs typically contain a plurality of individual zinc finger repeats and can each recognize a plurality of basepairs. The most common method to generate new zine-finger domain is to combine smaller zinc-finger “modules” of known specificity. The most common cleavage domain in ZENs is the non-specific cleavage domain from the type IIs restriction endonuclease FokI. Using the endogenous homologous recombination (HR) machinery and a homologous DNA template carrying CAR expression cassette, ZFNs can be used to insert the CAR expression cassette into genome. When the targeted sequence is cleaved by ZFNs, the HR machinery searches for homology between the damaged chromosome and the homologous DNA template, and then copies the sequence of the template between the two broken ends of the chromosome, whereby the homologous DNA template is integrated into the genome.

In certain embodiments, the TALEN system is used to modify the promoter/enhancer region of the IL-18 gene locus. Transcription activator-like effector nucleases (TALEN) are restriction enzymes that can be engineered to cut specific sequences of DNA. TALEN system operates on almost the same principle as ZENs. They are generated by combining a transcription activator-like effectors DNA-binding domain with a DNA cleavage domain. Transcription activator-like effectors (TALEs) are composed of 33-34 amino acid repeating motifs with two variable positions that have a strong recognition for specific nucleotides. By assembling arrays of these TALEs, the TALE DNA-binding domain can be engineered to bind desired DNA sequence, and thereby guide the nuclease to cut at specific locations in genome.

Methods for delivering the genome editing agents can vary depending on the need. In certain embodiments, the components of a selected genome editing method are delivered as DNA constructs in one or more plasmids. In certain embodiments, the components are delivered via viral vectors. Common delivery methods include but is not limited to, electroporation, microinjection, gene gun, impalefection, hydrostatic pressure, continuous infusion, sonication, magnetofection, adeno-associated viruses, envelope protein pseudotyping of viral vectors, replication-competent vectors cis and trans-acting elements, herpes simplex virus, and chemical vehicles (e.g., oligonucleotides, lipoplexes, polymersomes, polyplexes, dendrimers, inorganic Nanoparticles, and cell-penetrating peptides).

Modification can be made anywhere within the IL-18 gene locus, or anywhere that can influence gene expression of IL-18. In certain embodiments, the modification is introduced upstream of the transcriptional start site of IL-18 gene. In certain embodiments, the modification is introduced between the transcriptional start site and the protein coding region of IL-18 gene. In certain embodiments, the modification is introduced downstream of the protein coding region of IL-18 gene. In certain embodiments, the modification is introduced upstream of the transcriptional start site of IL-18 gene, wherein the modification supplies a new transcriptional start site.

### 7. Therapeutic Control

Therapeutic controls can be used to regulate cell proliferation, facilitate cell selection or a combination thereof. Examples of therapeutic controls include, but are not limited to, any one or more of truncated epidermal growth factor receptor (EGFRt), thymidine kinase, cytosine deaminase, nitroreductase, xanthine-guanine phosphoribosyl transferase, human caspase 8, human caspase 9, purine nucleoside phosphorylase, linamarase/linamarin/glucose oxidase, deoxyribonucleoside kinase, horseradish peroxidase (HRP)/indole-3-acetic (IAA), Gamma-glutamylcysteine synthetase, CD20/alphaCD20, CD34/thymidine kinase chimera, dox-depedent caspase-2, mutant thymidine kinase (HSV-TKSR39), AP1903/Fas system, a chimerie cytokine receptor (CCR), a selection marker, and combinations thereof. Examples of agents that regulate the therapeutic controls include, but are not limited to, any one or more of Herceptin, methotrexate, cetuximab, thymidine analogs (for example ganciclovir), (E)-5-(2-bromovinyl)-2′-deoxyuridine (BVDU), 5-flurocytosine (5-FC), 5-(azaridin-1-yl)-2,4-dinitrobenzamide (CB1954), 6-thioguanine, a synthetic dimerizing drug (for example API 903), fludarabine phosphate, linamarin (lin), nucleoside analogs (for example BVDU, difluorodeoxycytidine (dFdC), 1-β-D-arabinofuranosylthymine (ara-T)), indole-3-acetic (IAA), 1-buthionine-S,R-sulfoximine (BSO), rituximab (RTX), doxycycline, tyrosine kinase inhibitors or combinations thereof. These agents may be administered before, during or after the use of the therapeutic controls.

In certain embodiment, EGFRt may be used as an therapeutic control. In certain embodiment, EGFRt may be co-expressed with an antigen recognizing receptor to facilitate cell purification, in vivo or in vitro tracking, or regulation of cells by inducing cell ablation (see WO 2013123061 A1 and U.S. Pat. No. 8,802,374 B2, which is incorporated by reference in their entirety). Epidermal growth factor receptor (EGFR; ErbB-1, FIERI in humans) is a receptor tyrosine kinase which is not naturally expressed by hematopoietic or lymphopoietic cells. Extracellular domain of EGFR contains the binding sites of antibodies (e.g., cetuximab). In engineered immunoresponsive cell-based therapies, the use of EGFRt include, but is not limited to, ex vivo cell purification, in vivo cell tracking, and cell ablation. In certain embodiments, an antigen recognizing receptor and/or an IL-18 polypeptide of any aspect of the instant disclosure may be co-expressed with EGFRt. In certain embodiments, the antigen recognizing receptor and the therapeutic control can be constructed in a single, polycistronic expression cassette, in multiple expression cassettes of a single vector, or in multiple vectors. Examples of elements which create polycistronie expression cassette include, but is not limited to, various viral and non-viral Internal Ribosome Entry Sites and cleavable linkers disclosed in any aspect of the instant disclosure. In certain embodiments, the antigen recognizing receptor, IL-18 polypeptide, and EGFRt are co-expressed through a vector. Exemplary vectors include, but are not limited to, EGFRt-T2A-1928z-P2A-IL-18, EGFRt-T2A-19BB2-P2A-IL18 (wherein 4-1BB co-stimulatory domain is used), and EGFRt-T2A-19z-P2A-IL18 (first generation CAR with no co-stimulatory domain).

### 8. Polypeptides and Analogs

Also included in the presently disclosed subject matter are a CD19, CD28, CD3ζ, 4H1128z and IL-18 polypeptides or fragments thereof that are modified in ways that enhance their anti-neoplastic activity when expressed in an immunoresponsive cell. The presently disclosed subject matter provides methods for optimizing an amino acid sequence or nucleic acid sequence by producing an alteration in the sequence. Such alterations may include certain mutations, deletions, insertions, or post-translational modifications. The presently disclosed subject matter further includes analogs of any naturally-occurring polypeptide disclosed herein (including, but not limited to, CD19, CD28, CD3ζ, CD8, 4-1BB, 1928z, 4H1128z and IL-18). Analogs can differ from a naturally-occurring polypeptide disclosed herein by amino acid sequence differences, by post-translational modifications, or by both. Analogs can exhibit at least about 85%, about 90%, about 91%, about 92%, about 93%, about 94%, about 95%, about 96%, about 97%, about 98%, about 99% or more homologous to all or part of a naturally-occurring amino, acid sequence of the presently disclosed subject matter. The length of sequence comparison is at least 5, 10, 15 or 20 amino acid residues, preferably at least 25, 50, or 75 amino acid residues, and more preferably more than 100 amino acid residues. Again, in an exemplary approach to determining the degree of identity, a BLAST program may be used, with a probability score between e⁻³ and e⁻¹⁰⁰ indicating a closely related sequence. Modifications include in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally-occurring polypeptides of the invention by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989, or Ausubel et al., supra). Also included are cyclized peptides, molecules, and analogs which contain residues other than L-amina acids, e.g., D-amino acids or non-naturally occurring or synthetic amino acids, e.g., beta. or gamma. amino acids.

In addition to full-length polypeptides, the presently disclosed subject matter also provides fragments of any one of the polypeptides or peptide domains of the presently disclosed subject matter. As used herein, the term “a fragment” means at least 5, 10, 13, or 15 amino acids. In other embodiments a fragment is at least 20 contiguous amino acids, at least 30 contiguous amino acids, or at least 50 contiguous amino acids, and in other embodiments at least 60 to 80, 100, 200, 300 or more contiguous amino acids. Fragments of the invention can be generated by methods known to those skilled in the art or may result from normal protein processing (e.g., removal of amino acids from the nascent polypeptide that are not required for biological activity or removal of amino acids by alternative mRNA splicing or alternative protein processing events).

Non-protein analogs have a chemical structure designed to mimic the functional activity of a protein of the invention. Such analogs are administered according to methods of the presently disclosed subject matter. Such analogs may exceed the physiological activity of the original polypeptide. Methods of analog design are well known in the art, and synthesis of analogs can be carried out according to such methods by modifying the chemical structures such that the resultant analogs increase the anti-neoplastic activity of the original polypeptide when expressed in an immunoresponsive cell. These chemical modifications include, but are not limited to, substituting alternative R groups and varying the degree of saturation at specific carbon atoms of a reference polypeptide. In certain embodiments, the protein analogs are relatively resistant to in vivo degradation, resulting in a more prolonged therapeutic effect upon administration. Assays for measuring functional activity include, but are not limited to, those described in the Examples below.

### 9. Administration

Compositions comprising genetically modified immunoresponsive cells of the invention (e.g., T cells, NK cells, CTL cells, or their progenitors) can be provided systemically or directly to a subject for treating and/or preventing a neoplasia, pathogen infection, or infectious disease. In certain embodiments, cells of the presently disclosed subject matter are directly injected into an organ of interest (e.g., an organ affected by a neoplasia). Alternatively, compositions comprising the presently disclosed immunoresponsive cells are provided indirectly to the organ of interest, for example, by administration into the circulatory system (e.g., the tumor vasculature). Expansion and differentiation agents can be provided prior to, during or after administration of the cells to increase production of T cells, NK cells, or CTL cells in vitro or in vivo.

The modified cells can be administered in any physiologically acceptable vehicle, normally intravascularly, although they may also be introduced into bone or other convenient site where the cells may find an appropriate site for regeneration and differentiation (e.g., thymus). Usually, at least about 1×10⁵ cells will be administered, eventually reaching about 1×10¹⁰ or more. The presently disclosed immunoresponsive cells can comprise a purified population of cells. Those skilled in the art can readily determine the percentage of the presently disclosed immunoresponsive cells in a population using various well-known methods, such as fluorescence activated cell sorting (FACS). Suitable ranges of purity in populations comprising the presently disclosed immunoresponsive cells are about 50% to about 55%, about 5% to about 60%, and about 65% to about 70%. More preferably the purity is about 70% to about 75%, about 75% to about 80%, about 80% to about 85%; and still more preferably the purity is about 85% to about 90%, about 90% to about 95%, and about 95% to about 100%. Dosages can be readily adjusted by those skilled in the art (e.g., a decrease in purity may require an increase in dosage). The cells can be introduced by injection, catheter, or the like.

The presently disclosed compositions can be pharmaceutical compositions comprising the presently disclosed immunoresponsive cells or their progenitors and a pharmaceutically acceptable carrier. Administration can be autologous or heterologous. For example, immunoresponsive cells, or progenitors can be obtained from one subject, and administered to the same subject or a different. compatible subject. Peripheral blood derived immunoresponsive cells of the invention or their progeny (e.g., in vivo, ex vivo or in vitro derived) can be administered via localized injection, including catheter administration, systemic injection, localized injection, intravenous injection, or parenteral administration. When administering a therapeutic composition of the presently disclosed subject matter (e.g., a pharmaceutical composition containing a presently disclosed immunoresponsive cell), it can be formulated in a unit dosage injectable form (solution, suspension, emulsion).

### 10. Formulations

Compositions comprising the presently disclosed immunoresponsive cells can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the genetically modified immunoresponsive cells utilized in practicing the present invention in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the presently disclosed subject matter, however, any vehicle, diluent, or additive used would have to be compatible with the genetically modified immunoresponsive cells or their progenitors.

The compositions can be isotonic, i.e., they can have the same osmotic pressure as blood and lacrimal fluid. The desired isotonicity of the compositions of this presently disclosed subject matter may be accomplished using sodium chloride, or other pharmaceutically acceptable agents such as dextrose, boric acid, sodium tartrate, propylene glycol or other inorganic or organic solutes. Sodium chloride is preferred particularly for buffers containing sodium ions.

Viscosity of the compositions, if desired, can be maintained at the selected level using a pharmaceutically acceptable thickening agent. Methylcellulose is preferred because it is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

Those skilled in the art will recognize that the components of the compositions should be selected to be chemically inert and will not affect the viability or efficacy of the genetically modified immunoresponsive cells as described in the presently disclosed subject matter. This will present no problem to those skilled in chemical and pharmaceutical principles, or problems can be readily avoided by reference to standard texts or by simple experiments (not involving undue experimentation), from this disclosure and the documents cited herein.

One consideration concerning the therapeutic use of the presently disclosed immunoresponsive cells is the quantity of cells necessary to achieve an optimal effect. The quantity of cells to be administered will vary for the subject being treated. In certain embodiments, between about 10⁴ and about 10¹⁰, between about 10⁵ and about 10⁹, or between about 10⁶ and about 10⁸ genetically presently disclosed cells are administered to a human subject. More effective cells may be administered in even smaller numbers. In some embodiments, at least about 1×10⁸, about 2×10⁸, about 3×10⁸, about 4×10⁸, or about One consideration concerning the therapeutic use of the presently disclosed immunoresponsive cells is the quantity of cells necessary to achieve an optimal effect. The quantity of cells to be administered will vary for the subject being treated. In a one embodiment, between about 10⁴ and about 10¹⁰, between about 10⁵ and about 10⁹, or between about 10⁶ and about 10⁸ presently disclosed immunoresponsive cells are administered to a human subject. More effective cells may be administered in even smaller numbers. In some embodiments, at least about 1×10⁸, about 2×10⁸, about 3×10⁸, about 4×10⁴, or about 5×10⁸ presently disclosed immunoresponsive cells are administered to a human subject. The precise determination of what would be considered an effective dose may be based on factors individual to each subject, including their size, age, sex, weight, and condition of the particular subject. Dosages can be readily ascertained by those skilled in the art from this disclosure and the knowledge in the art.

The skilled artisan can readily determine the amount of cells and optional additives, vehicles, and/or carrier in compositions and to be administered in methods of the invention. Typically, any additives (in addition to the active cell(s) and/or agent(s)) are present in an amount of 0.001 to 50% (weight) solution in phosphate buffered saline, and the active ingredient is present in the order of micrograms to milligrams, such as about 0.0001 to about 5 wt %, preferably about 0.0001 to about 1 wt %, still more preferably about 0.0001 to about 0.05 wt % or about 0.001 to about 20 wt %, preferably about 0.01 to about 10 wt %, and still more preferably about 0.05 to about 5 wt %. Of course, for any composition to be administered to an animal or human, and for any particular method of administration, it is preferred to determine therefore: toxicity, such as by determining the lethal dose (LD) and LD50 in a suitable animal model e.g., rodent such as mouse; and, the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

### 11. Methods of Treatment

The presently disclosed subject matter provides methods for increasing an immune response in a subject in need thereof. The presently disclosed immunoresponsive cells and compositions comprising thereof can be used for treating and for preventing a neoplasia in a subject. The presently disclosed immunoresponsive cells and compositions comprising thereof can also be used for treating and/or preventing a pathogen infection or other infectious disease in a subject, such as an immunocompromised human subject. The methods comprise administering the presently disclosed immunoresponsive cells in an amount effective to achieve the desired effect, be it palliation of an existing condition or prevention of recurrence. For treatment, the amount administered is an amount effective in producing the desired effect. An effective amount can be provided in one or a series of administrations. An effective amount can be provided in a bolus or by continuous perfusion.

An “effective amount” (or, “therapeutically effective amount”) is an amount sufficient to effect a beneficial or desired clinical result upon treatment. An effective amount can be administered to a subject in one or more doses. In terms of treatment, an effective amount is an amount that is sufficient to palliate, ameliorate, stabilize, reverse or slow the progression of the disease, or otherwise reduce the pathological consequences of the disease. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the immunoresponsive cells administered.

For adoptive immunotherapy using antigen-specific T cells, cell doses in the range of about 10⁶-10¹⁰ (e.g., about 10⁹) are typically infused. Upon administration of the presently disclosed cells into the host and subsequent differentiation, T cells are induced that are specifically directed against the specific antigen. “Induction” of T cells can include inactivation of antigen-specific T cells such as by deletion or anergy. Inactivation is particularly useful to establish or reestablish tolerance such as in autoimmune disorders. The modified cells can be administered by any method known in the art including, but not limited to, intravenous, subcutaneous, intranodal, intratumoral, intrathecal, intrapleural, intraperitoneal and directly to the thymus.

The presently disclosed subject matter provides methods for treating and/or preventing a neoplasia in a subject. The method can comprise administering an effective amount of the presently disclosed immunoresponsive cells or a composition comprising thereof to a subject having a neoplasia.

Non-limiting examples of neoplasia include blood cancers (e.g. leukemias, lymphomas, and myelomas), ovarian cancer, breast cancer, bladder cancer, brain cancer, colon cancer, intestinal cancer, liver cancer, lung cancer, pancreatic cancer, prostate cancer, skin cancer, stomach cancer, glioblastoma, throat cancer, melanoma, neuroblastoma, adenocarcinoma, glioma, soft tissue sarcoma, and various carcinomas (including prostate and small cell lung cancer). Suitable carcinomas further include any known in the field of oncology, including, but not limited to, astrocytoma, fibrosarcoma, myxosarcoma, liposarcoma, oligodendroglioma, ependymoma, medulloblastoma, primitive neural ectodermal tumor (PNET), chondrosarcoma, osteogenic sarcoma, pancreatic ductal adenocarcinoma, small and large cell lung adenocarcinomas, chordoma, angiosarcoma, endotheliosarcoma, squamous cell carcinoma, bronchoalveolarcarcinoma, epithelial adenocarcinoma, and liver metastases thereof, lymphangiosarcoma, lymphangioendotheliosarcoma, hepatoma, cholangiocarcinoma, synovioma, mesothelioma, Ewing's tumor, rhabdomyosarcoma, colon carcinoma, basal cell carcinoma, sweat gland carcinoma, papillary carcinoma, sebaceous gland carcinoma, papillary adenocarcinoma, cystadenocarcinoma, medullary carcinoma, bronchogenie carcinoma, renal cell carcinoma, bile duct carcinoma, choriocarcinoma, seminoma, embryonal carcinoma, Wilms' tumor, testicular tumor, medulloblastoma, craniopharyngioma, ependymoma, pinealoma, hemangioblastoma, acoustic neuroma, oligodendroglioma, meningioma, neuroblastoma, retinoblastoma, leukemia, multiple myeloma, Waldenstrom's macroglobulinemia, and heavy chain disease, breast tumors such as ductal and lobular adenocarcinoma, squamous and adenocarcinomas of the uterine cervix, uterine and ovarian epithelial carcinomas, prostatic adenocarcinomas, transitional squamous cell carcinoma of the bladder, B and T cell lymphomas (nodular and diffuse) plasmacytoma, acute and chronic leukemias, malignant melanoma, soft tissue sarcomas and leiomyosarcomas. In certain embodiments, the neoplasia is selected from the group consisting of blood cancers (e.g. leukemias, lymphomas, and myelomas), ovarian cancer, prostate cancer, breast cancer, bladder cancer, brain cancer, colon cancer, intestinal cancer, liver cancer, lung cancer, pancreatic cancer, prostate cancer, skin cancer, stomach cancer, glioblastoma, and throat cancer. In certain embodiments, the presently disclosed immunoresponsive cells and compositions comprising thereof can be used for treating and/or preventing blood cancers (e.g., leukemias, lymphomas, and myelomas) or ovarian cancer, which are not amenable to conventional therapeutic interventions.

The subjects can have an advanced form of disease, in which case the treatment objective can include mitigation or reversal of disease progression, and/or amelioration of side effects. The subjects can have a history of the condition, for which they have already been treated, in which case the therapeutic objective will typically include a decrease or delay in the risk of recurrence.

Suitable human subjects for therapy typically comprise two treatment groups that can be distinguished by clinical criteria. Subjects with “advanced disease” or “high tumor burden” are those who bear a clinically measurable tumor. A clinically measurable tumor is one that can be detected on the basis of tumor mass (e.g., by palpation, CAT scan, sonogram, mammogram or X-ray; positive biochemical or histopathologic markers on their own are insufficient to identify this population). A pharmaceutical composition embodied in this invention is administered to these subjects to elicit an anti-tumor response, with the objective of palliating their condition. Ideally, reduction in tumor mass occurs as a result, but any clinical improvement constitutes a benefit. Clinical improvement includes decreased risk or rate of progression or reduction in pathological consequences of the tumor.

A second group of suitable subjects is known in the art as the “adjuvant group.” These are individuals who have had a history of neoplasia, but have been responsive to another mode of therapy. The prior therapy can have included, but is not restricted to, surgical resection, radiotherapy, and traditional chemotherapy. As a result, these individuals have no clinically measurable tumor. However, they are suspected of being at risk for progression of the disease, either near the original tumor site, or by metastases. This group can be further subdivided into high-risk and low-risk individuals. The subdivision is made on the basis of features observed before or after the initial treatment. These features are known in the clinical arts, and are suitably defined for each different neoplasia. Features typical of high-risk subgroups are those in which the tumor has invaded neighboring tissues, or who show involvement of lymph nodes.

Another group have a genetic predisposition to neoplasia but have not yet evidenced clinical signs of neoplasia. For instance, women testing positive for a genetic mutation associated with breast cancer, but still of childbearing age, can wish to receive one or more of the immunoresponsive cells described herein in treatment prophylactically to prevent the occurrence of neoplasia until it is suitable to perform preventive surgery.

As a consequence of surface expression of a receptor that binds to a tumor antigen and a secretable IL-18 polypeptide (e.g., exogenous IL-18 polypeptide) that enhances the anti-tumor effect of the immunoresponsive cell, adoptively transferred human T or NK cells are endowed with augmented and selective cytolytic activity at the tumor site. Furthermore, subsequent to their localization to tumor or viral infection and their proliferation, the T cells turn the tumor or viral infection site into a highly conductive environment for a wide range of immune cells involved in the physiological anti-tumor or antiviral response (tumor infiltrating lymphocytes, NK-, NKT-cells, dendritic cells, and macrophages).

Additionally, the presently disclosed subject matter provides methods for treating and/or preventing a pathogen infection (e.g., viral infection, bacterial infection, fungal infection, parasite infection, or protozoal infection) in a subject, e.g., in an immunocompromised subject. The method can comprise administering an effective amount of the presently disclosed immunoresponsive cells or a composition comprising thereof to a subject having a pathogen infection. Exemplary viral infections susceptible to treatment using a method of the invention include, but are not limited to, Cytomegalovirus (CMV), Epstein Barr Virus (EBV), Human Immunodeficiency Virus (HIV), and influenza virus infections.

### 12. Kits

The presently disclosed subject matter provides kits for treating and or preventing a neoplasia or a pathogen infection. In certain embodiments, the kit comprises a therapeutic or prophylactic composition comprising an effective amount of presently disclosed immunoresponsive cells. In some embodiments, the kit comprises a sterile container; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments. In certain non-limiting embodiments, the kit includes an isolated nucleic acid encoding an antigen recognizing receptor (e.g., a CAR or a TCR) directed toward an antigen of interest and an isolated nucleic acid encoding an IL-18 polypeptide in expessible (and secretable) form, which may optionally be comprised in the same or different vectors.

If desired the immunoresponsive cell and/or nucleic acid is provided together with instructions for administering the cell or nucleic acid to a subject having or at risk of developing a neoplasia or pathogen or immune disorder. The instructions will generally include information about the use of the composition for the treatment and/or prevention of neoplasia or a pathogen infection. In certain embodiments, the instructions include at least one of the following: description of the therapeutic agent; dosage schedule and administration for treatment or prevention of a neoplasia, pathogen infection, or immune disorder or symptoms thereof; precautions; warnings; indications; counter-indications; over-dosage information; adverse reactions; animal pharmacology; clinical studies; and/or references. The instructions may be printed directly on the container (when present), or as a label applied to the container, or as a separate sheet, pamphlet, card, or folder supplied in or with the container.

### EXAMPLES

The practice of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”. second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### Example 1. The Interleukin-18 (IL-18) Secreting CAR-T Cells

### Introduction

A genetically modified Chimeric Antigen Receptor (CAR-T cell) that constitutively secretes Interleukin-18 (IL-18) was generated for the treatment of malignancies. IL-18 can be used in any type of T cell adoptive therapy technology, including CAR-T cells. TCRs and Tumor Infiltrating Lymphocytes (TILs).

In the specific setting of CD19+ tumor cells, this new technology presents major improvements when compared to 1928z CAR-T cells. The new construct has shown improved results in vivo and has significantly prolonged survival curves in human and murine models.

This product is useful for the treatment of any CD19+ malignancy, including Acute Lymphoblastic Leukemia (ALL), Chronic Lymphocytic Leukemia (CLL) and B-cell Lymphomas. Further, this technology can be expanded to treat other malignancies, either solid or hematological, including Acute Myeloid Leukemia (AML), Multiple Myeloma, ovarian, breast, lung, brain and prostate cancers.

### Results

### Human IL18 Secreting CAR T Cells Prolong Survival in Xenograft Scid-Beige Mouse Model

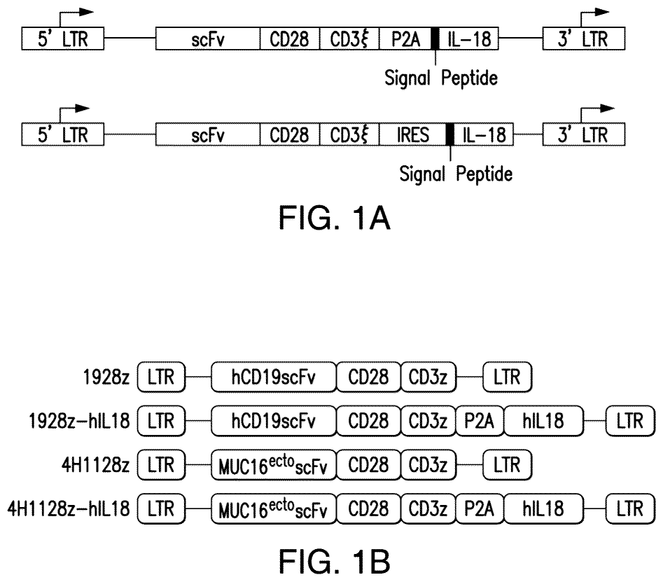

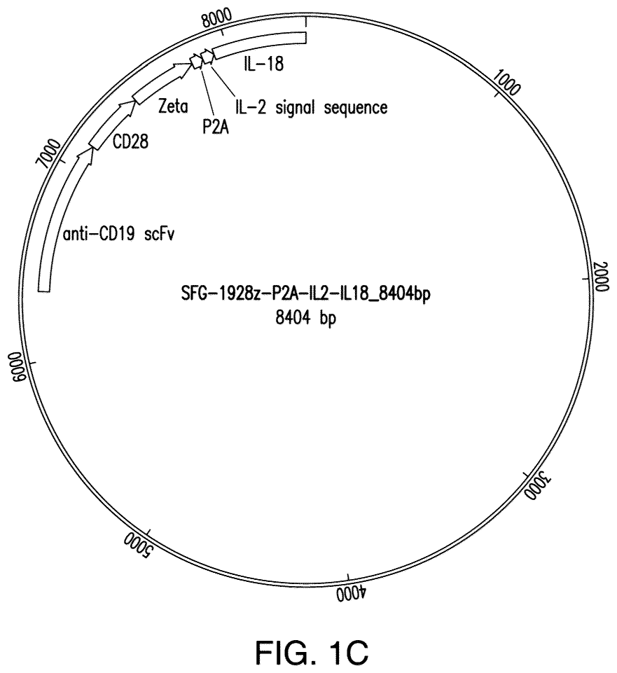

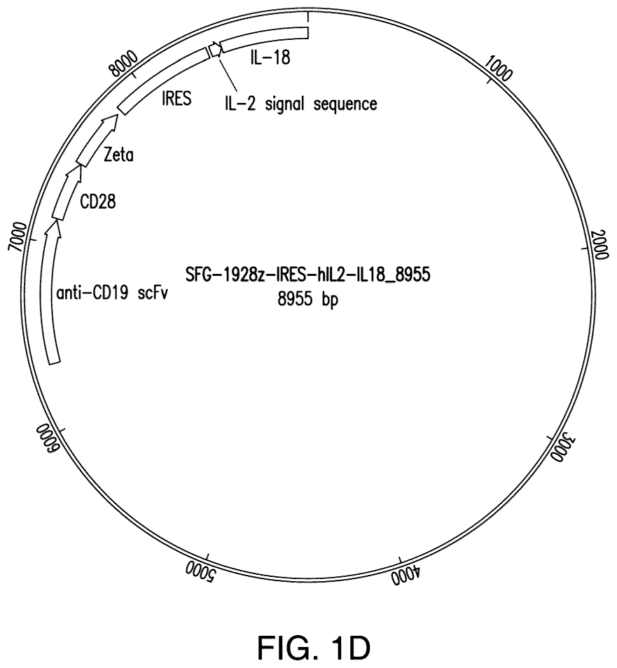

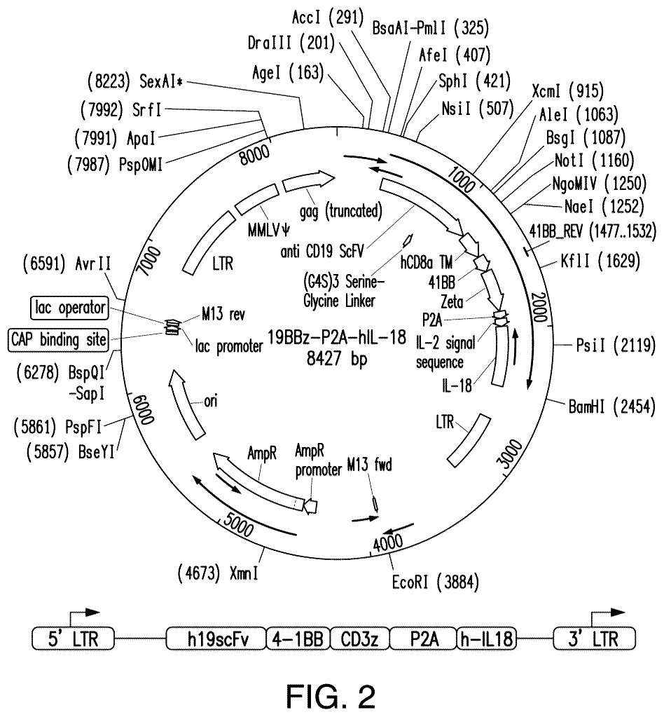

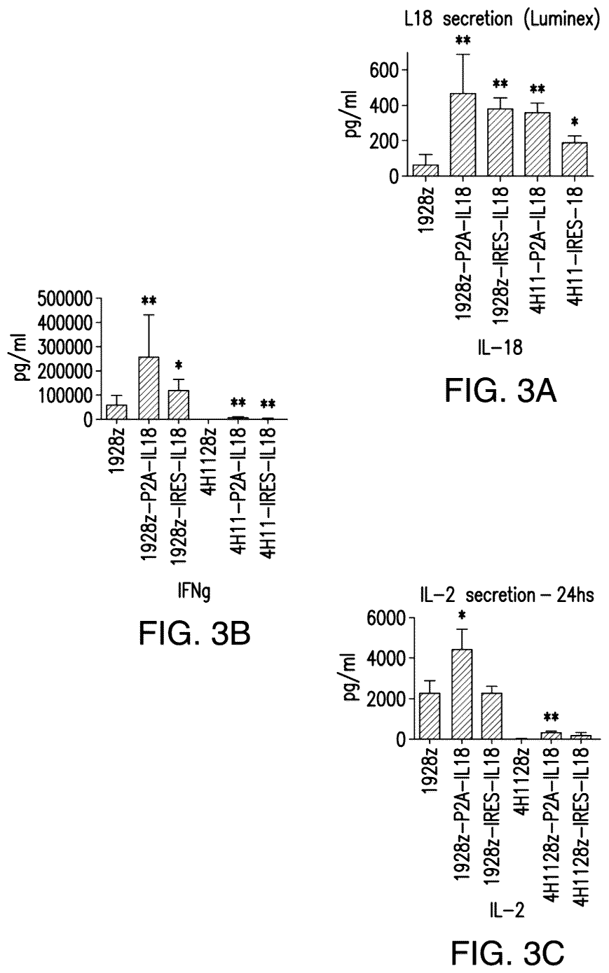

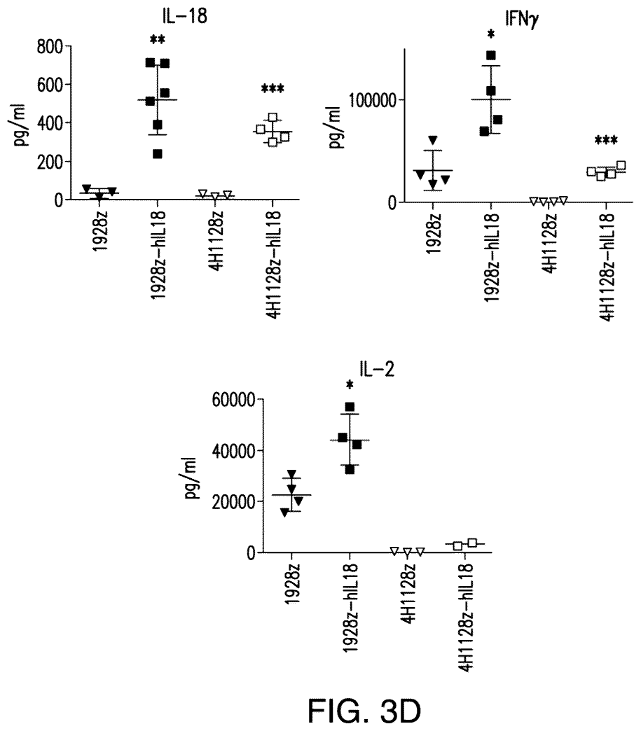

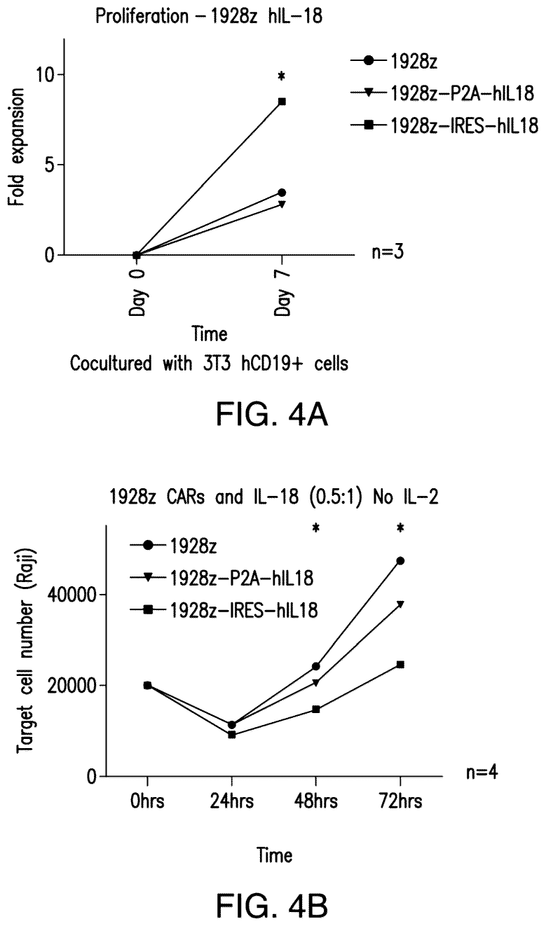

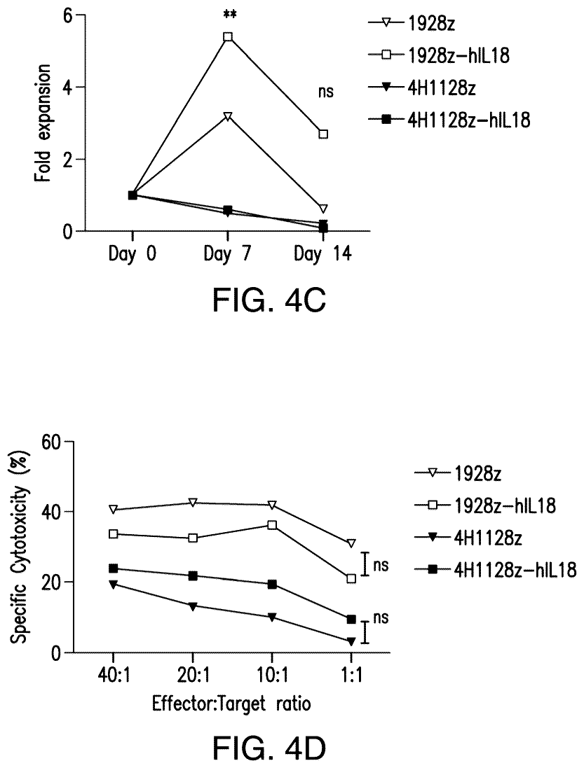

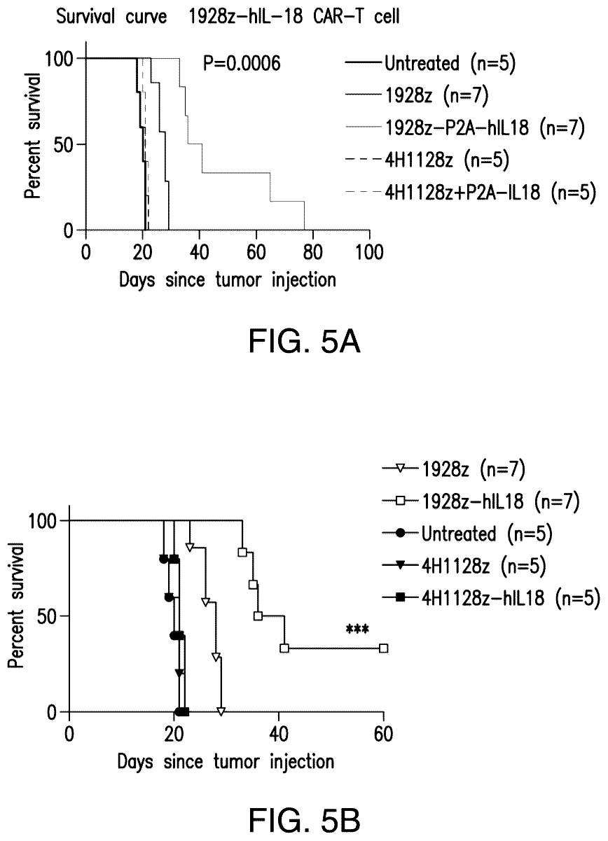

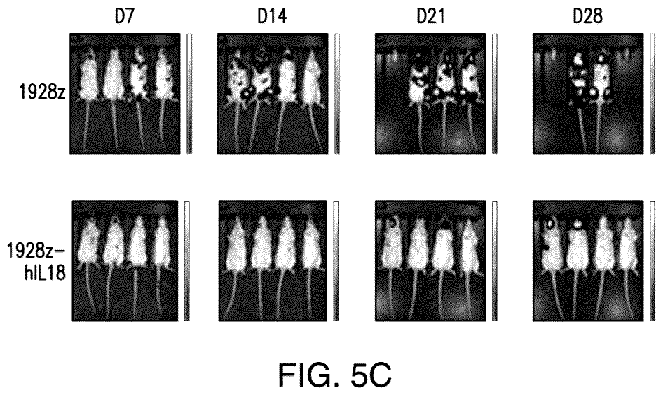

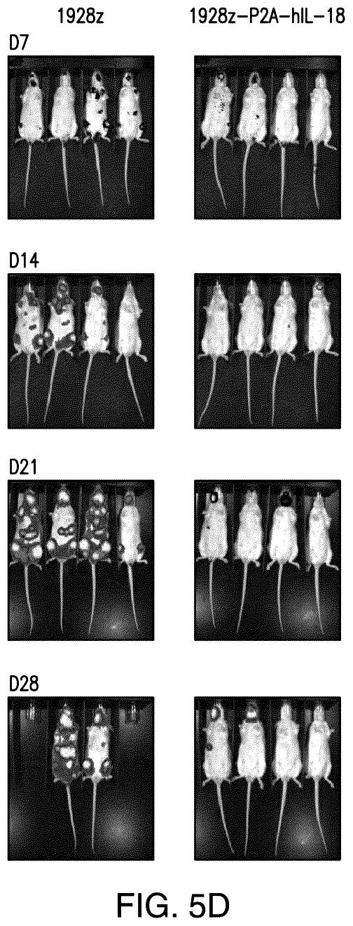

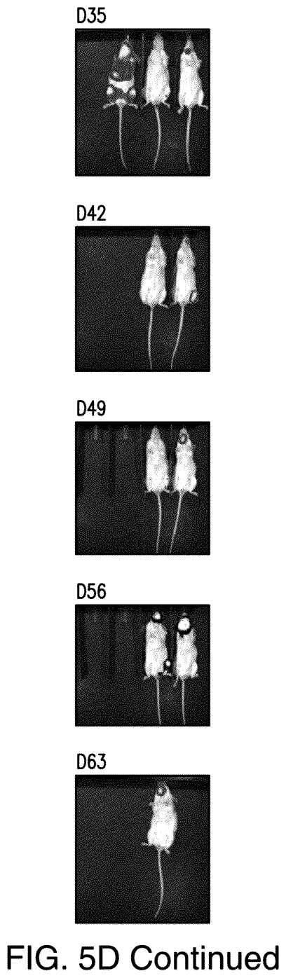

Human CD19-directed 1928z-hIL18 CAR retroviral construct were derived from a previously described and clinically utilized 1928z CAR construct. Ovarian tumor-targeted anti-Muc16^{ecto} 4H1128z CAR T cells were utilized as untargeted controls (FIG. 1). 19BBz-IL18 CAR T cell constructs were also generated (FIG. 2) In order to validate the construct, human T cells modified to express the 1928z-hIL18 CAR vector were compared to 1928z CAR T cells and demonstrated enhanced in vitro IL-18 (p=0.003), IFNγ (p=0.01) and IL-2 (p=0.01) secretion after stimulation with CD19⁺ NALM6 B-ALL tumor cells (FIG. 3). In addition, 1928z-hIL18 CAR T cells compared to 1928z CAR T cells demonstrated enhanced proliferation after repeated NALM6 tumor stimulation (p=0.003) and retained anti-tumor cytotoxicity (FIG. 4). In order to assess the in vivo anti-tumor efficacy of 1928z-hIL18 CAR T cells, NALM6-GFP⁺/Luc⁺ tumor-bearing Scid-Beige mice were treated with 1928z or 1928z-hIL18 CAR. T cells. In this model, mice were treated with low dose CAR T cells to better observe the survival benefits of the 1928z-hIL18 CAR T cells therapy. 19287-hIL18 CAR T cells significantly enhanced survival when compared to 1928z CAR T cells (p=0.0006) and significantly lowered tumor burden as assessed by bioluminescent imaging (ROI) (FIG. 5).

Human IL-18 DNA sequence, including an human IL-2 signal peptide having a nucleic acid sequence set forth in SEQ ID NO: 32, which is provided below:

(SEQ ID NO: 32)
CCATGGGTTACAGGATGCAACTCCTGTCTTGCATTGCACTAAGTCTTGCA
CTTGTCACAAACAGTGGCTACTTTGGCAAGCTTGAATCTAAATTATCAGT
CATAAGAAATTTGAATGACCAAGTTCTCTTCATTGACCAAGGAAATCGGC
CTCTATTTGAAGATATGACTGATTCTGACTGTAGAGATAATGCACCCCGG
ACCATATTTATTATAAGTATGTATAAAGATAGCCAGCCTAGAGGTATGGC
TGTAACTATCTCTGTGAAGTGTGAGAAAATTTCAACTCTCTCCTGTGAGA
ACAAAATTATTTCCTTTAAGGAAATGAATCCTCCTGATAACATCAAGGAT
ACAAAAAGTGACATCATATTCTTTCAGAGAAGTGTCCCAGGACATGATAA
TAAGATGCAATTTGAATCTTCATCATACGAAGGATACTTTCTAGCTTGTG
AAAAAGAGAGAGACCTTTTTAAACTCATTTTGAAAAAAGAGGATGAATTG
GGGGATAGATCTATAATGTTCACTGTTCAAAACGAAGACtag GGATCC

### Murine IL-18 Secreting CAR T Cells Enhance Survival of EL4hCD19+Tumor-Bearing Syngeneic Immuno-Competent Mice in the Absence of Chemotherapy Preconditioning

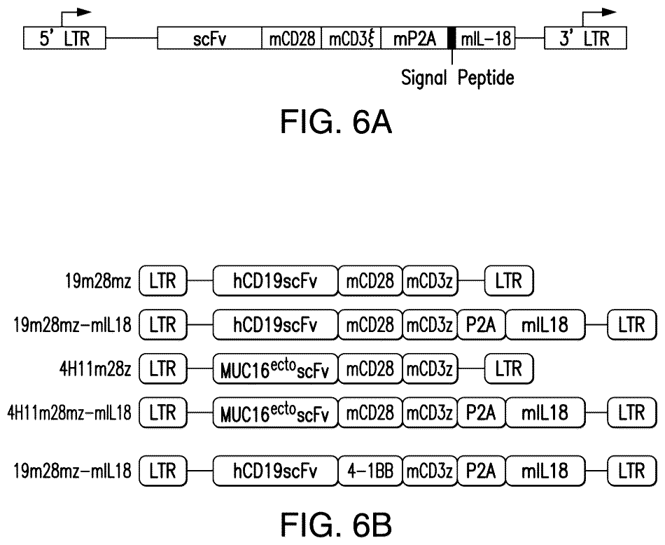

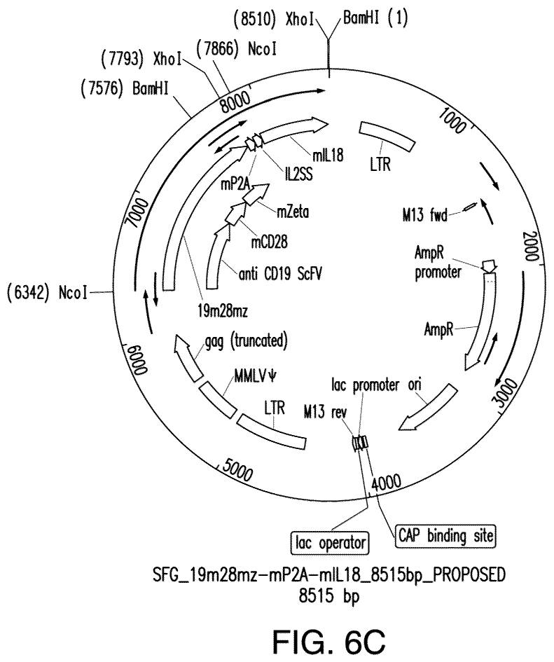

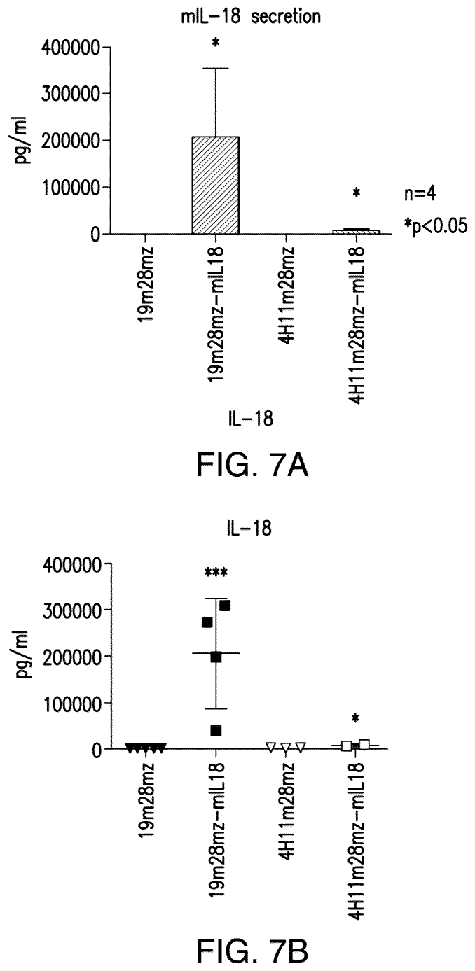

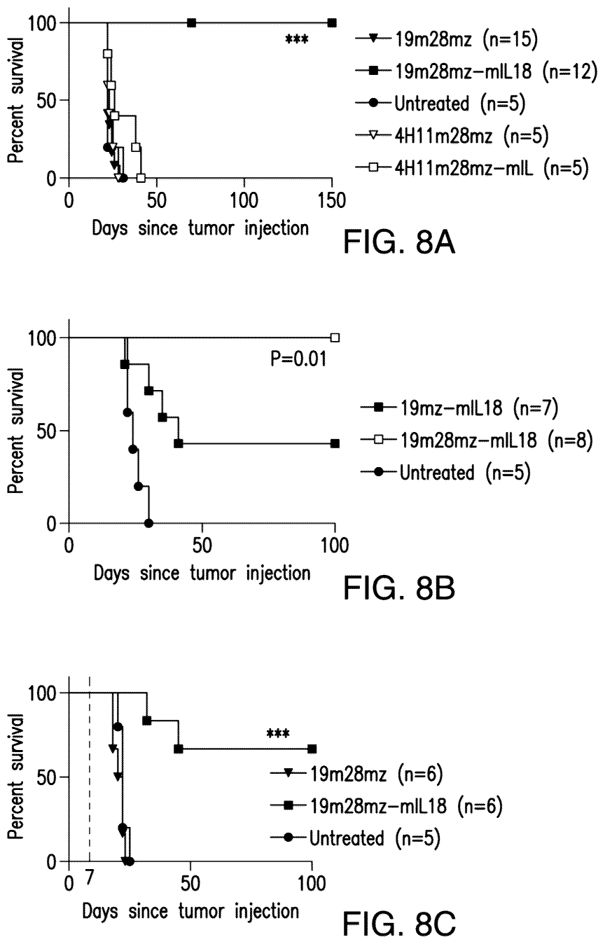

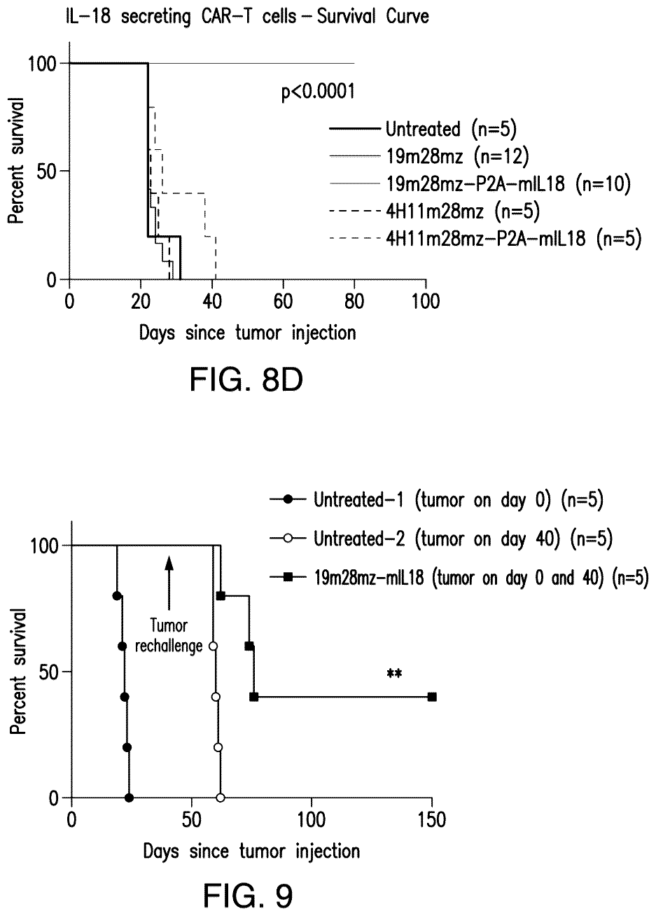

Given the fact that an immunocompromised xenotransplant mouse tumor model has limited clinical and biological relevance, IL-18 secreting CAR T cells were next studied in the context of syngeneic, immune competent models of disease wherein a more comprehensive analysis of the anti-tumor effects mediated by murine IL-18 (mIL-18) secreting T cells could be assessed. To this end, a panel of retroviral vectors encoding murine CAR T cells that target hCD19 were generated: 19m28mz-mIL18, 19m28mz-mIL12, 19mz-mIL, 18, 19mDel-mIL18 and 19mDel all derived from a 19m28mz retroviral construct. Anti-Muc16^{ecto} 4H11m28mz-mIL18 and 4H11m28mz target an ovarian antigen and were used as untargeted controls(37) (FIG. 6). As predicted, mouse T cells modified with the 19m28mz-mIL18 retroviral vector demonstrated enhanced in vitro IL-18 secretion (p=0.005) (FIG. 7). Utilizing a syngeneic hCD19⁺ transgenie mouse model (C57BL/6 mCD19^{+/-} hCD19^{+/-}), the efficacy of 19m28mz-mIL18 CAR T cells were evaluated in mice infused systemically with a thymoma tumor cell line (EL4), modified to express hCD19 (EL4hCD19⁺). Based on our previously published results, syngeneic mice inoculated I.V. with EL4hCD19⁺ tumor cells on day 0 and treated anti-hCD19 CAR T cells on day 1 failed to eradicate tumor cells in the absence of prior cyclophosphamide preconditioning and all mice succumbed to disease. This aggressive tumor model was ideal to investigate whether 19m28mz-mIL18 CAR T cells could overcome the lack of anti-tumor efficacy exhibited by T cells modified to express the 19m28mz alone. In the absence of preconditioning, 19m28mz-mIL18 CAR T cells (2.5×10⁶ CAR cells/mouse) were capable of significantly enhancing long-term survival of EL4hCD19⁺ tumor-bearing syngeneic mice, compared to 19m28mz CAR T cell treatment (p<0.0001) (FIG. 8-A). IL-18 secreting first-generation CAR T cells (19mz-mIL18) also demonstrated enhanced anti-tumor effect and significantly increased mice long-term survival, compared to 19m28mz CAR T cells and controls (FIG. 8-B). Furthermore, 19m28mz-mILIS CAR T cells were also capable of enhancing long-term survival in delayed tumor models wherein CAR T cells (2.5×10⁶ CAR T cells/mouse) were administered on day 7 after tumor inoculation, when compared to mice treated with 19m28mz CAR T cells (p=0.0009) (FIG. 8-C). Whether 19m28mz-mIL. 18 CAR T cells persist and retain meaningful anti-tumor efficacy against tumor re-challenge were next assessed. To this end, surviving mice previously inoculated with 1×10⁶ EL4hCD19⁺ tumor cells on day 0 and treated with 19m28mz-mIL18 CAR T cells on day 1 were re-challenged with a second inoculum of 1×10⁶ tumor cells 40 days after the first tumor injection. Mice treated with 19m28mz-mIL18 CAR T cells were capable of rejecting a second lethal dose of tumor (p=0.004) (FIG. 9). Significantly, mice that succumbed to disease in these studies harbored CD19⁺ tumor cells on necropsy.

Murine IL˜18 DNA sequence, including an IL-2 signal peptide having the nucleic acid sequence set forth in SEQ ID NO: 33, which is provided below:

(SEQ ID NO: 32)
CTCGAGGGTAGCGGTGCCACTAACTTCAGTCTCCTTAAGCAGGCTGGCGA
TGTGGAAGAAAATCCTGGACCAtCCATGGGTTACAGGATGCAACTCCTGT
CTTGCATTGCACTAAGTCTTGCACTTGTCACAAACAGTGGCGCTGCCATG
TCAGAAGACTCTTGCGTCAACTTCAAGGAAATGATGTTTATTGACAACAC
GCTTTACTTTATACCTGAAGAAAATGGAGACCTGGAATCAGACAACTTTG
GCCGACTTCACTGTACAACCGCAGTAATACGGAATATAAATGACCAAGTT
CTCTTCGTTGACAAAAGACAGCCTGTGTTCGAGGATATGACTGATATTGA
TCAAAGTGCCAGTGAACCCCAGACCAGACTGATAATATACATGTACAAAG
ACAGTGAAGTAAGAGGACTGGCTGTGACCCTCTCTGTGAAGGATAGTAAA
ATGTCTACCCTCTCCTGTAAGAACAAGATCATTTCCTTTGAGGAAATGGA
CCCACCTGAAAATATTGATGATATACAAAGTGATCTCATATTCTTTCAGA
AACGTGTTCCAGGACACAACAAGATGGAGTTTGAATCTTCACTGTATGAA
GGACACTTTCTTGCTTGCCAAAAGGAAGATGATGCTTTCAAACTCATTCT
GAAAAAAAAGGATGAAAATGGGGATAAATCTGTAATGTTCACTCTCACTA
ACTTACATCAAAGTTAGCTCGAGGATCC

### Murine IL-18 Secreting CAR T Cells Exhibit Enhanced Expansion and Persistence, and Induce Prolonged B-Cell Aplasia Dependent Upon Autocrine IL-18R Signaling

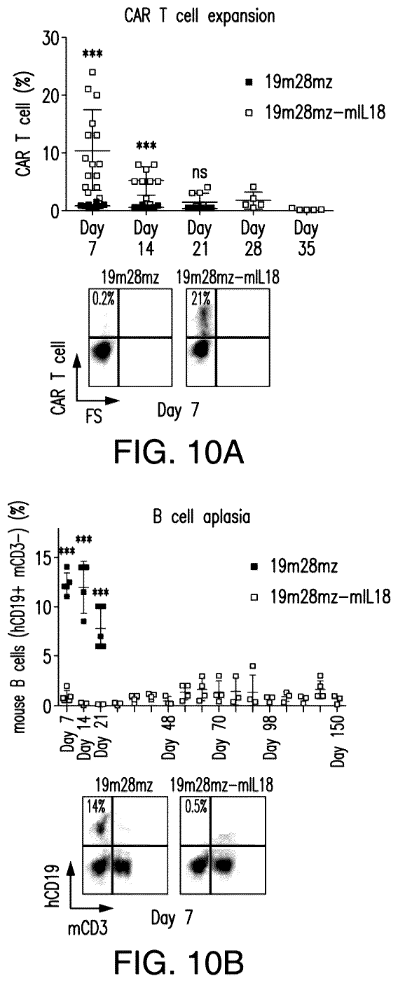

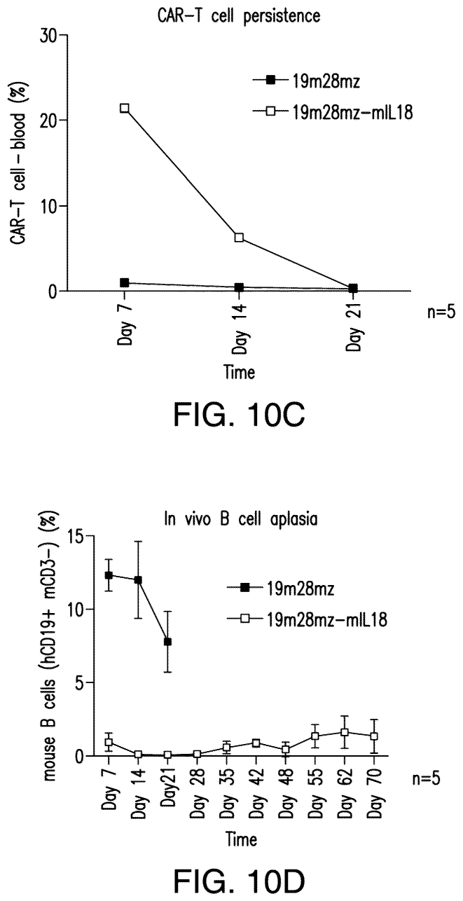

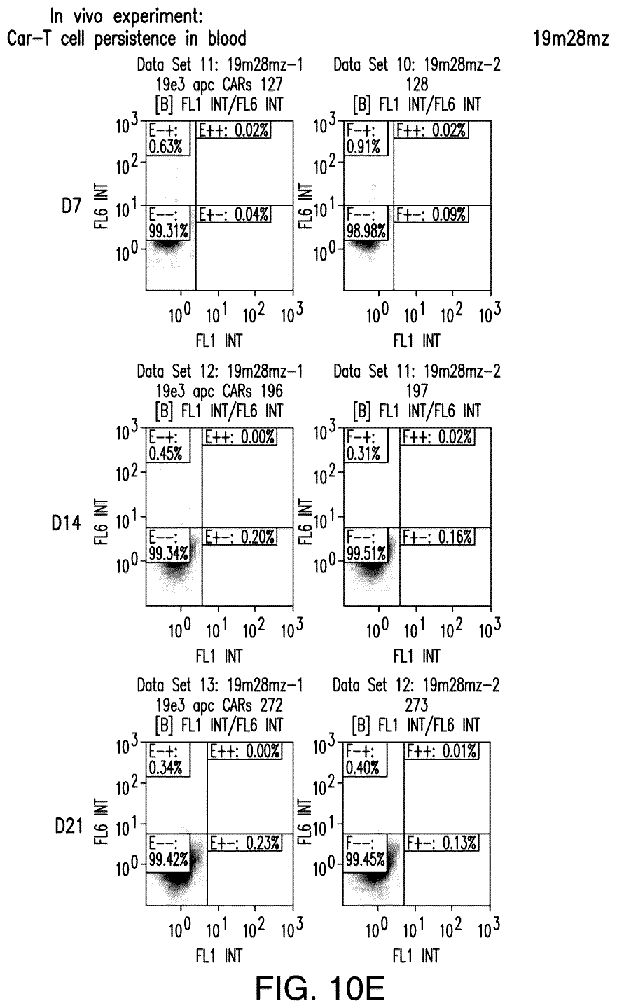

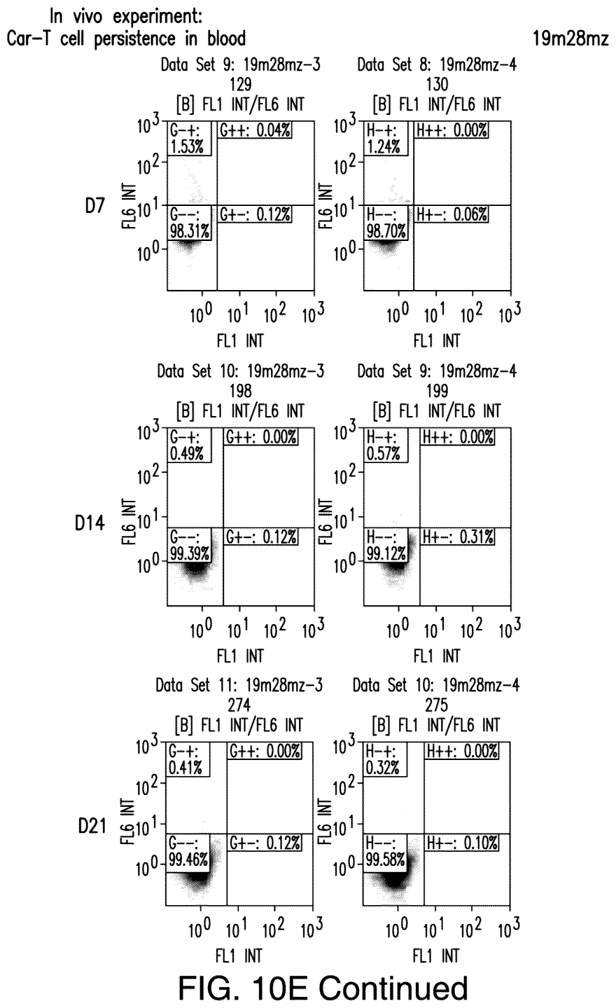

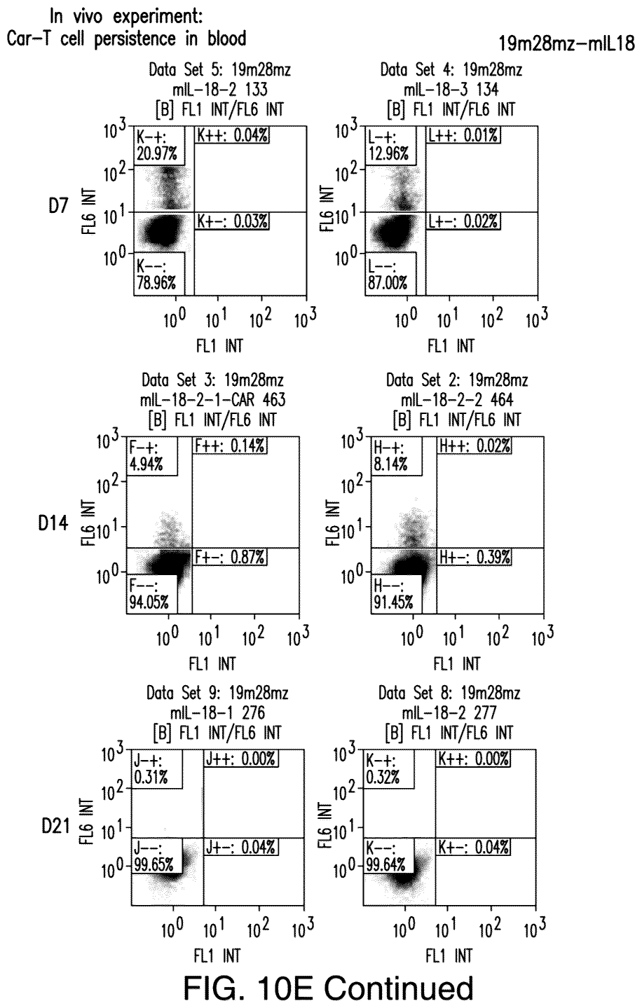

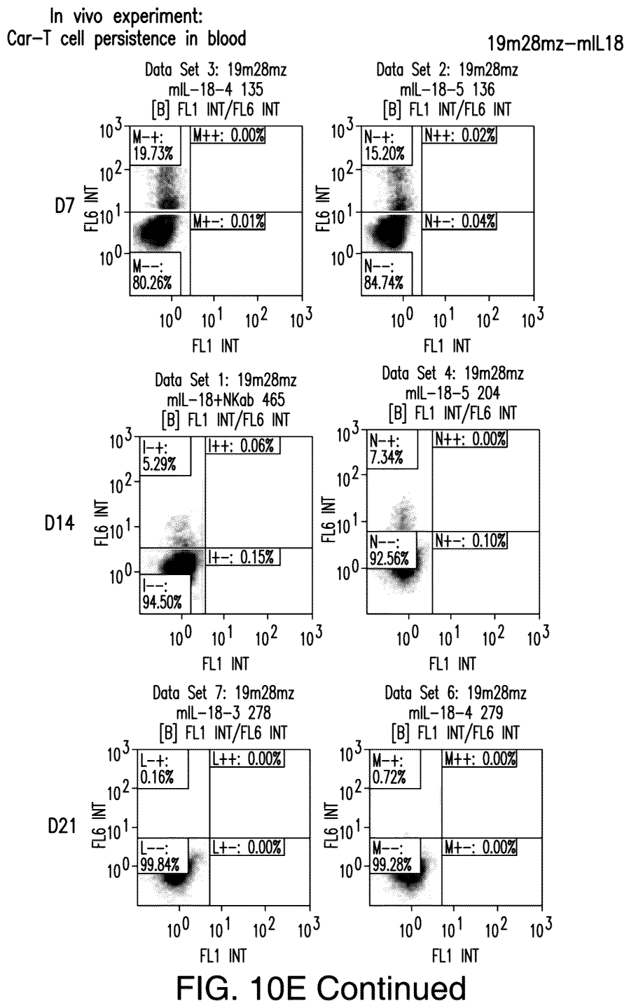

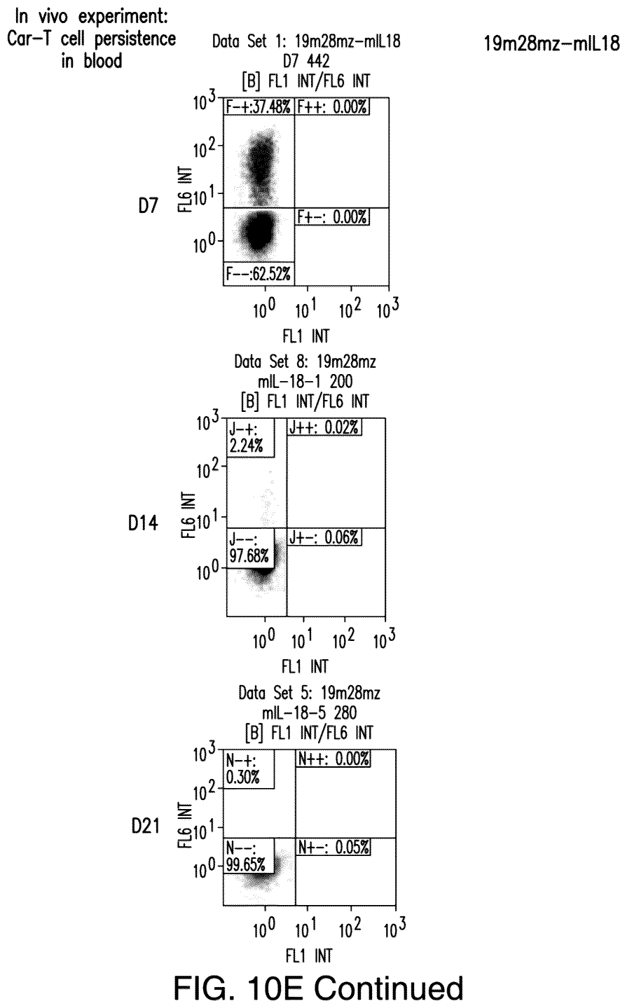

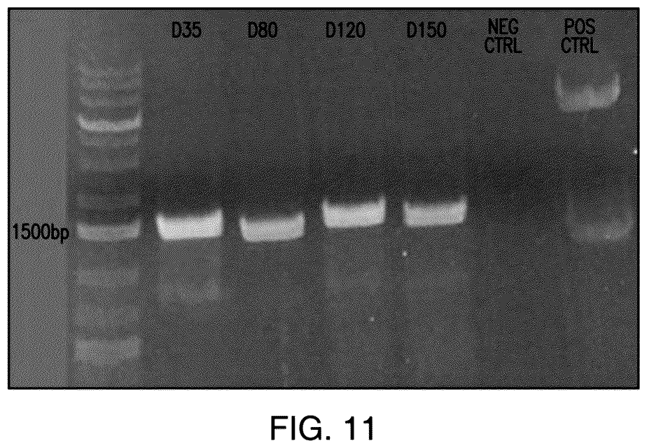

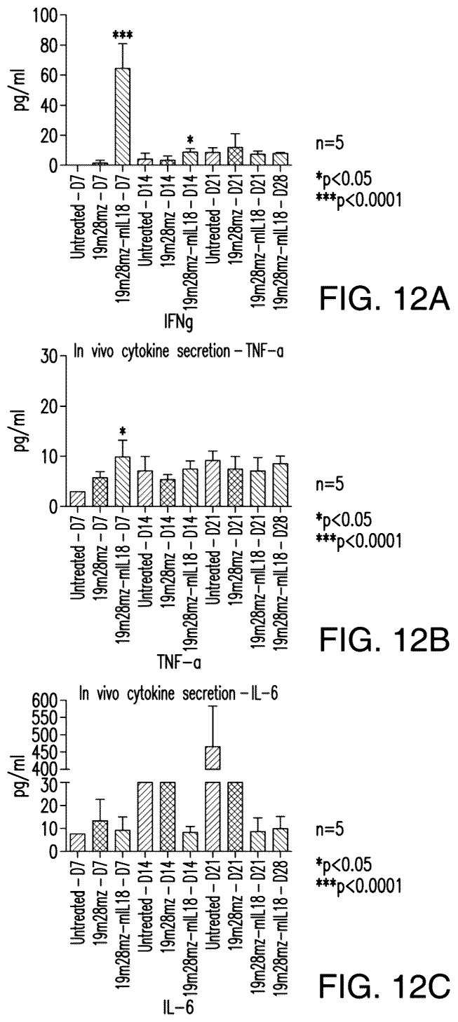

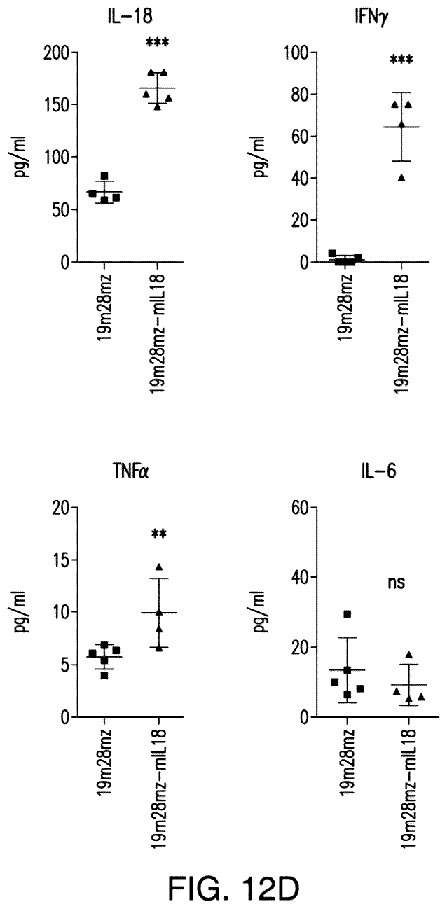

19m28mz-mIL18 CAR T cells displayed enhanced in vivo expansion and were detected in peripheral blood by flow cytometry for up to 28 days after infusion, whereas 19m28mz CAR T cells were not detected in peripheral blood at any time point tested (FIG. 10). hCD19⁺ transgenic mice were previously shown to develop B cell aplasia after treatment with CD19-directed CAR T cells. B cell aplasia in this mouse model is an effective surrogate marker for CD19 targeted CAR T cell activity and directly correlates with the presence of CAR T cells either in circulation or in the bone marrow. Mice treated with 19m28mz-mIL18 CAR T cells developed relative and persistent B cell aplasias for up to 150 days after treatment whereas mice treated with 19m28mz CAR T cells did not develop B cell aplasias at any time point (FIG. 10). To further verify persistence of 19m28mz-mIL18 CAR T cells over time and validate the role of these CAR T cells in observed prolonged B cell aplasias, bone marrow aspirates were collected from 19m28mz-mIL18 CAR T cell treated mice and analyzed for detection of the CAR construct utilizing polymerase chain reaction (PCR) technique. 19m28mz-mIL18 CAR T cells were detected by PCR in the bone marrow at 35, 80, 120 and 150 days after infusion (FIG. 11), suggesting that the prolonged B cell aplasia was directly related to the enhanced persistence of CAR T cells in the bone marrow. In addition, 19m28mz-mIL18 CAR T cells were capable of significantly enhancing serum levels of IL-18, IFNγ and TNFα at day 7, compared to 19m28mz CAR T cells (p<0.0001, p<0.0001, p=0.003, respectively) (FIG. 12). Significantly, no increase in serum IL-6, a cytokine associated with cytokine release syndrome (CRS) in the clinical setting, was detected in mice treated with 19m28mz-mIL18 CAR T cells. (FIG. 12).

### IL-18 secreting CAR T cells activate endogenous immune cells

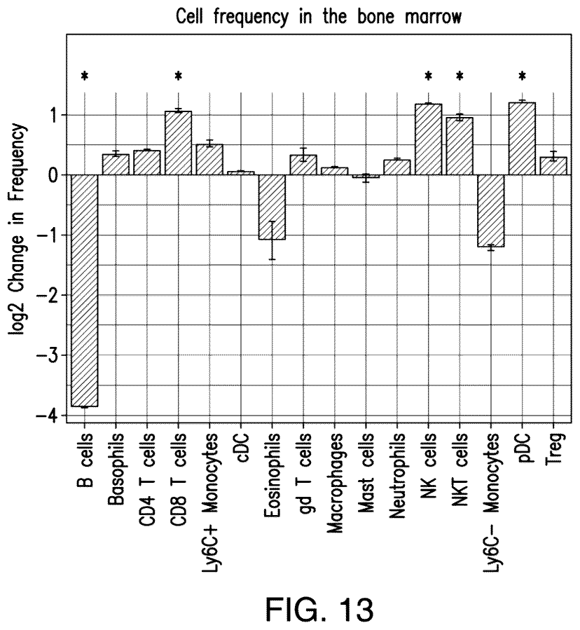

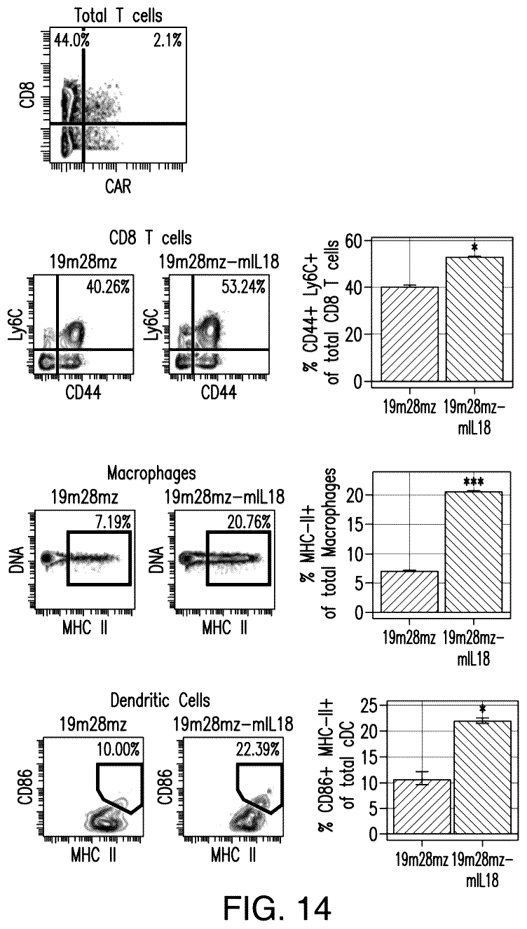

In order to evaluate the 19m28mz-mIL18 CAR T cells migration capacity to the tumor site as well as its effects on endogenous immune cells, experiments were performed in immunocompetent syngeneic mice and mass cytometry technology (CyTOF) was utilized to analyze bone marrow samples. Bone marrow analysis demonstrated that 19m28mz-mIL18 CAR T cells were indeed capable of not only significantly decreasing the B cell population but also of inducing expansion of bone marrow endogenous immune effector cells, such as NK cells, NKT cells, dendritic cells (DC) and endogenous CD8 T cells, compared to 19m28mz CAR T cell treated mice (p=0.03; p=0.03; p=0.03; p=0.03; p=0.03, respectively) (FIG. 13). The increased CD8 T cell population in the bone marrow was mostly composed of endogenous CD8 non-CAR T cells (FIG. 14). More interestingly, 19m28mz-mIL18 CAR T cells were capable of modulating and activating endogenous immune cells residing in the bone marrow. Mice treated with 19m28mz-mIL18 CAR T cells, when compared to mice treated with 19m28mz CAR T cells, presented enhanced numbers of endogenous CD8 T cells with a central memory phenotype (CD44⁺;Ly6C⁺) (p=0.01), macrophages with an M1 phenotype (MHC-II+) (p<0.0001) and dendritic cells with a more mature and activated phenotype (CD86+;MHC-II+) (p=0.02) (FIG. 14). Also, while many endogenous and CAR CD8 T cells exhibited characteristics of central memory cells (CD44+; Ly6C+), CAR T cells expressed higher levels of CD27, PD-1 and CD3 while endogenous T cells expressed higher levels of Ly6C, CD8 and CD90 (FIG. 14).

### IL-18 Secreting CAR T Cells Recruit Endogenous Anti-Tumor Immune Effector Cells

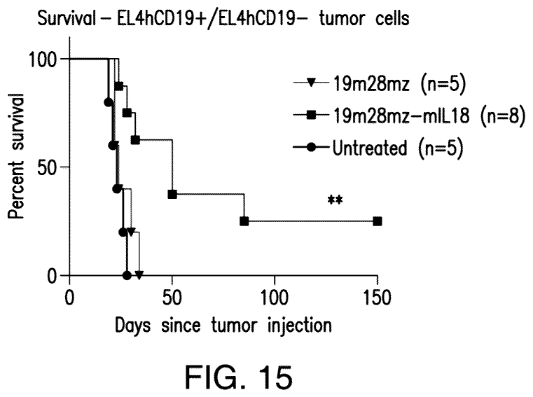

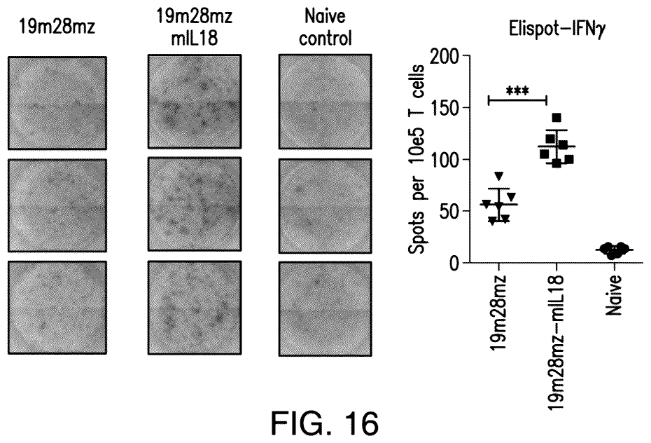

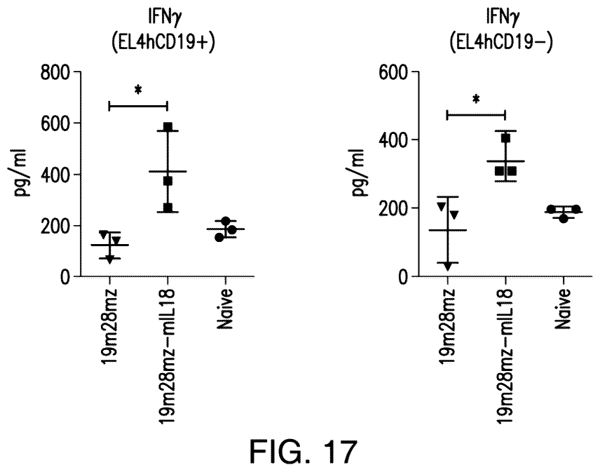

It was postulated that 19m28mz-mIL18 CAR T cells could possibly be stimulating endogenous CD8 T cells towards a central memory phenotype and thus enhancing the anti-tumor effect through recruitment of endogenous CAR⁻ tumor targeted T cells. In order to confirm this hypothesis syngeneic mice were inoculated with a mixed population of equal amounts (1×10⁶ cells/mouse) of EL4hCD19⁺ and EL4hCD19⁻ tumor cells treated with anti-CD19 19m28mz-mIL18 CAR T cells. Interestingly, 19m28mz-mIL18 CAR T cells were capable of enhancing long-term survival of mice inoculated with both CD19⁺ and CD19⁻ tumor cells at a 1:1 ratio (FIG. 15), consistent with the recruitment of endogenous CD8 T cells targeted to antigens other than CD19 expressed by CD19⁺ tumor cells. To further verify that 19m28mz-mIL18 CAR T cells recruit endogenous tumor targeted T cells, additional Elispot experiments were conducted with FACS sorted CAR⁻ splenocytes derived from EL4hCD19⁺ tumor-bearing mice treated with either 19m28mz or 19m28mz-mIL18 CAR T cells. Elispot results demonstrated that isolated CAR⁻ splenocytes derived from mice treated with 19m28mz-mIL18 CAR T cells exhibited enhanced levels of IFNγ in the presence of EL4hCD19⁺ tumor cells, compared to 19m28mz CAR T cells (p=0.0009) (FIG. 16). To corroborate these results, similar experiments were conducted utilizing CAR⁻ splenocytes derived from mice treated with Thy1.1-derived CAR T cells injected into EL4hCD19⁺ tumor-bearing syngeneic mice. Thy1.1 negative splenocytes were FACS sorted, cocultured in vitro with either EL4hCD19⁺ or EL4hCD19⁻ tumor cells, and supernatant was collected for IFNγ cytokine quantification. Cytokine results demonstrated that CAR⁻ splenocytes derived from mice treated with 19m28mz-mIL18 CAR T cells were capable of secreting increased amounts of IFNγ in both EL4hCD19⁺ or EL4hCD19⁻ (FIG. 17) coculture experiments (p=0.04 and p=0.03, respectively). Thus, demonstrating that 19m28mz-mIL18 CAR T cells are indeed capable of recruiting endogenous anti-tumor immune effector cells.

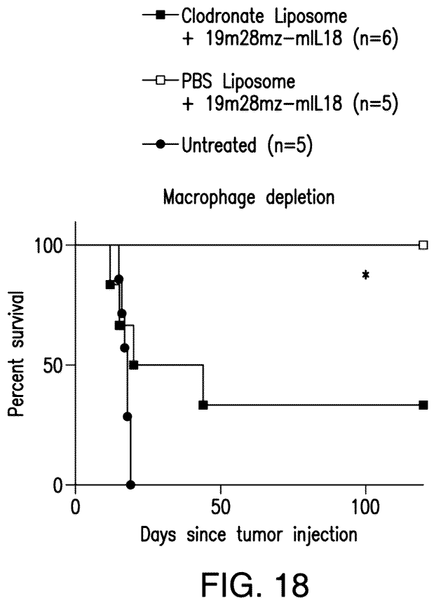

Next, to investigate the role of other endogenous immune cell and their potential contribution to long-term survival after IL-18 CAR therapy, host macrophages were depleted. Depletion of murine macrophages prior to inoculation with EL4hCD19⁺ tumor cells and treatment with 19m28mz-mIL18 CAR T cells led to significant decrease in long-term survival, compared to mice without macrophage depletion (p=0.03) (FIG. 18). These results demonstrate that 19m28mz-mIL18 CAR T cells activate macrophages in the bone marrow and that macrophages display significant anti-tumor activity and act in combination with the CAR T cells to optimally eradicate tumor cells in vivo.

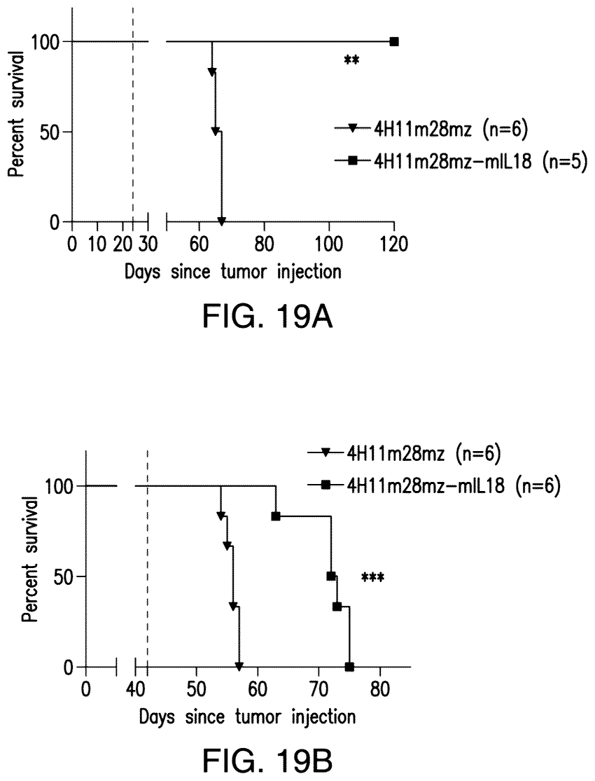

In order to confirm that the findings were not restricted to CD19⁺ hematological malignancies, the IL-18 CAR T cell studies were expanded to an ovarian carcinoma solid tumor model. To this end, murine retroviral constructs of the previously published CAR to the truncated MUC16 antigen (MUC16^{ecto}) 4H11 CAR(37, 38) were generated and in vivo experiments. C57BL/6 syngeneic mice were inoculated with ID8 (MUC16^{ecto}) ovarian tumor cells and treated with anti-MUC16^{ecto} 4H11m28mz and 4H11m28mz-mIL18 CAR T cells. Our results demonstrated that anti-MUC16^{ecto} 4H11m28mz-mIL18 CAR T cells were capable of significantly enhancing ovarian tumor-bearing syngeneic mice long-term survival in both low and high tumor burden models (FIG. 19). These studies verify the potential application of this IL-18 secreting adoptive CAR T cell approach to both liquid and solid tumor malignancies.

### TCR/IL-18 T Cells Display Enhanced Anti-Tumor Effect In Vitro and In Vivo

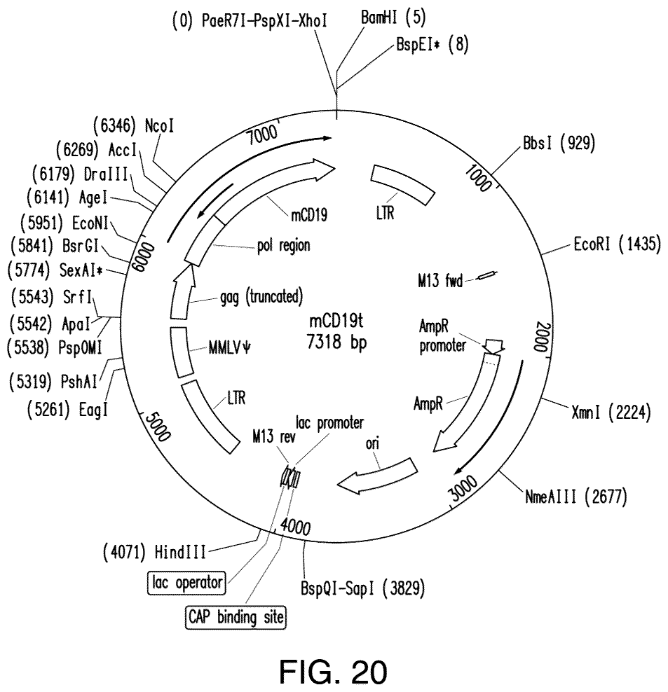

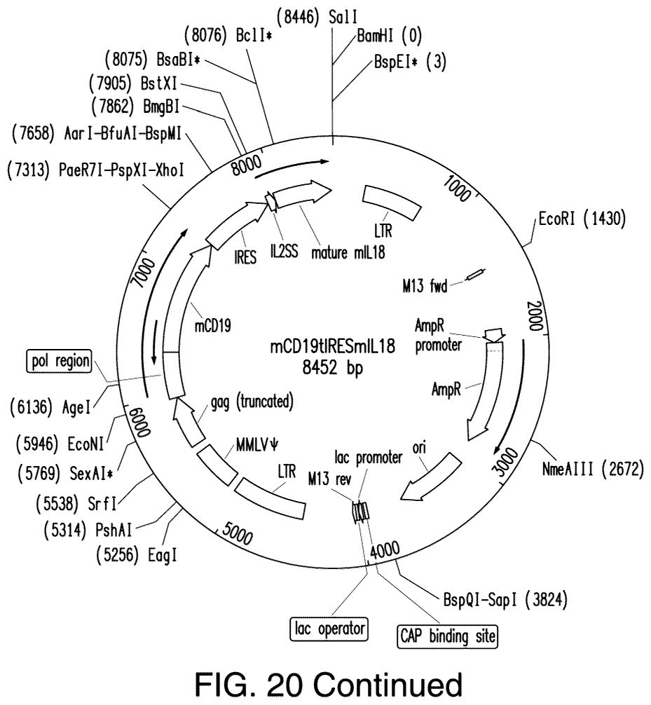

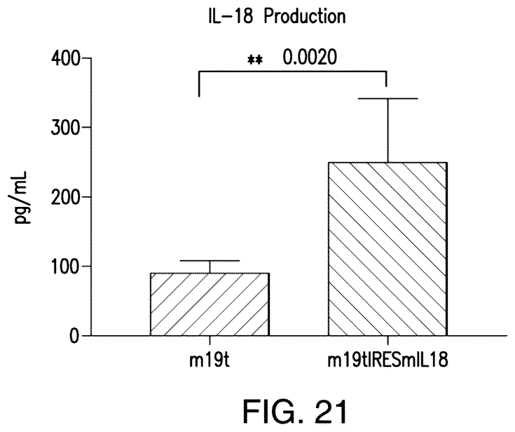

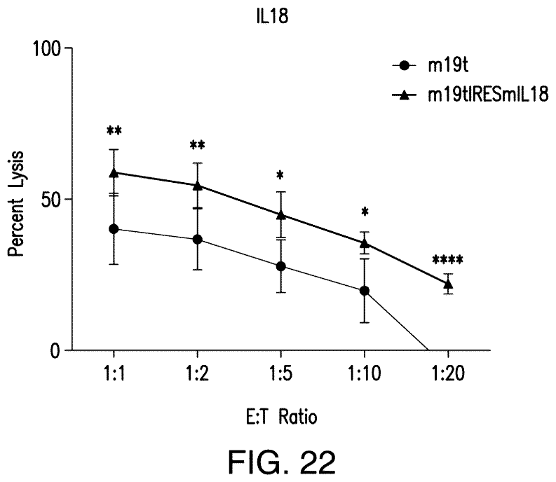

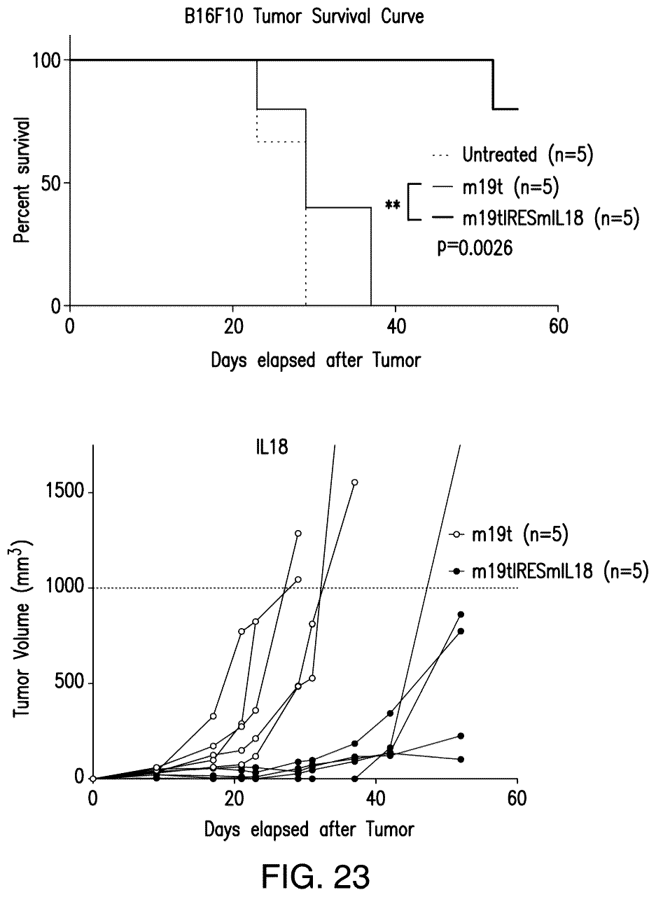

To generate the Interleukin-18 armored pmel-1 TCR T cells, first a bicistronic retroviral vector was engineered with a truncated form of the mouse CD19 protein (mCD19t) in the first position and mature murine IL-18 in the second position in a retroviral simian foamy virus (SFG) retroviral vector. The truncated mouse CD19 gene contained only the extracellular part of the protein and did not express the signaling domain, making it a non-functional protein to be used as a transduction marker. A negative control vector was generated that contains only the mCD19t gene (mCD19t construct). This construct acted as a transduction and experimental control (FIG. 20). Pmel-1 TCR/IL-18 T cells were shown to actively secrete IL-18 in vitro after 24 hours coculture with B16F10 melanoma tumor cells (FIG. 21). The in vitro cytotoxic potential of armored IL-18 pmel-1 TCR T cells on B16F10 tumor cells that were transduced to express a GFP/luciferase gene was then analyzed and compared to the mCD19t control pmel-1 T cells. The modified pmel-1 T cells were co-cultured at various Effector to Target ratios with the gp100 positive B16F10 melanoma tumor cells for 24 hours. After 24 hours, the luciferin substrate was added and the luminescence of the cells was measured and used to calculate percent lysis of the tumor cells by the T cells (FIG. 22). An in vivo model of gp100+, B16F10 melanoma was utilized to test the efficacy of the IL-18 armored pmel-1 TCR T cells in eradicating tumors and enhancing survival. Subcutaneous injections of the B16F10 tumor line into C57BL/6 mice at 1×10⁵ cells/mouse established the disease model. Pmel-1 directed T cells harvested from the spleens of B6.Cg-Thy1^{a}/CyTg(TcraTcrb)8Rest/J mice (The Jackson Laboratory) and transduced with the bicistronic engineered armor vector containing mIL-18 or the control mCD19t construct were injected intravenously by tail vein into the C57BL/6 mice 10 days after tumor. These mice were monitored for disease progression via tumor volume as well as anti-tumor response. Mice exhibiting tumors larger than 1 cm³ were sacrificed. Differences in survival and tumor volumes between the experimental groups were shown in FIG. 23.

### Embodiments of the Invention

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A method of reducing tumor burden in a subject, the method comprising administering an effective amount of an immunoresponsive cell comprising (a) a chimeric antigen receptor (CAR) that binds to an antigen and (b) an exogenous and constitutively secretable IL-18 polypeptide or a fragment thereof, thereby inducing tumor cell death in the subject.

2. The method of claim 1, wherein the method reduces the number of tumor cells, reduces tumor size, and/or eradicates the tumor in the subject.

3. The method of claim 1, wherein the subject has not received preconditioning therapy.

4. A method of treating and/or preventing neoplasia, the method comprising administering an effective amount of an immunoresponsive cell comprising (a) a chimeric antigen receptor (CAR) that binds to an antigen, and (b) an exogenous and constitutively secretable IL-18 polypeptide or a fragment thereof, thereby treating and/or preventing a neoplasia in the subject.

5. The method of claim 4, wherein the neoplasia is selected from the group consisting of blood cancer, B cell leukemia, multiple myeloma, lymphoblastic leukemia (ALL), chronic lymphocytic leukemia, non-Hodgkin's lymphoma, and ovarian cancer.

6. The method of claim 4, wherein the neoplasia is B cell leukemia, multiple myeloma, lymphoblastic leukemia (ALL), chronic lymphocytic leukemia, or non-Hodgkin's lymphoma, and the antigen is CD19.

7. The method of claim 4, wherein the neoplasia is ovarian, and the antigen is MUC16.

8. The method of claim 4, wherein the subject has not received preconditioning therapy.

9. The method of claim 4, wherein the exogenous IL-18 polypeptide comprises a heterologous signal sequence at the amino-terminus.

10. The method of claim 9, wherein said heterologous signal sequence is selected from the group consisting of IL-2 signal sequence, the kappa leader sequence, the CD8 leader sequence, and combinations thereof.

11. The method of claim 4, wherein the exogenous IL-18 polypeptide enhances an immune response of the immunoresponsive cell.

12. The method of claim 4, wherein the exogenous IL-18 polypeptide increases immune-activating cytokine production.

13. The method of claim 12, wherein the immune-activating cytokine is selected from the group consisting of IL-2, TNF-α and IFN-γ.

14. The method of claim 4, wherein the CAR is recombinantly expressed.

15. The method of claim 4, wherein the CAR is expressed from a vector.

16. The method of claim 4, wherein the cell is selected from the group consisting of a T cell, a Natural Killer (NK) cell, a cytotoxic T lymphocyte (CTL), a regulatory T cell, a Natural Killer T (NKT) cell, a human embryonic stem cell, and a pluripotent stem cell from which lymphoid cells may be differentiated.

17. The method of claim 16, wherein the cell is a T cell.

18. The method of claim 4, wherein the method reduces or eradicates the tumor burden in the subject.
